(19) United States
(12) Patent Application Publication
(10) Pub. No.: US 2026/0216086 A1
(43) Pub. Date: Jul. 30, 2026

(54) INTERTUMORAL AND INTRATUMORAL DELIVERY OF CYTOKINES USING MESOPOROUS SILICA RODS AS AN IMMUNOMODULATING SYSTEM

(71) Applicant: Attivare Therapeutics Inc., Natick, MA (US)

(72) Inventors: Jessica MCDONOUGH, Malden, MA (US); Edward DOHERTY, Mansfield, MA (US); Benjamin T. SEILER, Hopkinton, MA (US); Chyenne YEAGER, Boston, MA (US); Everton MANDLEY, Providence, RI (US); Fernanda LANGELLOTTO, Newton, MA (US); Robert PIERCE, Natick, MA (US)

(73) Assignee: Attivare Therapeutics Inc., Natick, MA (US)

(21) Appl. No.: 19/146,486

(22) PCT Filed: Jan. 8, 2024
(86) PCT No.: PCT/US2024/010752

§ 371 Date: Jul. 8, 2025

Related U.S. Application Data
(60) Provisional application No. 63/437,747, filed on Jan. 8, 2023.
(60) Provisional application No. 63/599,507, filed on Nov. 15, 2023.

(51) Int. Cl. A61K 9/51; A61K 38/20; A61P 35/00; C07K 16/28

(52) U.S. Cl. CPC A61K 9/5115; A61K 38/2013; A61K 38/208; A61P 35/00; C07K 16/2818

(57) ABSTRACT

Embodiments of the invention include methods of treating conditions that are ameliorated by stimulating an immune response. In aspects, the methods include injection of mesoporous silica rods (MSRs) at or near an affected tissue. The MSRs can include a cytokine (e.g., IL-2 or IL-12) and/or an adjuvant. The MSRs can induce an innate immune response to treat cancer, infection and other ailments. The methods can include administering an additional medicament such as an immune checkpoint inhibitor.

### CROSS REFERENCE TO RELATED APPLICATIONS

The present application claims priority to U.S. provisional patent application No. 63/437,747 filed Jan. 8, 2023 and U.S. provisional patent application No. 63/599,507 filed May 15, 2023, the contents of which are incorporated by reference herein.

### FIELD OF THE INVENTION

The invention relates to cancer therapeutics, and more specifically, to structures and methods of treating cancer using mesoporous silica material to deliver cytokines.

### BACKGROUND

The immune system of an organism acts in coordination with myriad biological processes to protect itself from diseases, disorders and ailments. The immune system carries out these processes by detecting and responding to a wide variety of pathogens and foreign objects not native to the body of the organism.

Dysfunction of the immune system can lead to several diseases, disorders and ailments, including autoimmune diseases, inflammatory diseases and cancer. Other ailments afflicting an immune system are immunodeficiencies. Immunodeficiency occurs when the immune system's activity and response falls below typically healthy thresholds and can result in recurring and life-threatening infections. Immunodeficiency can manifest as the result of genetic disease, or acquired conditions such as HIV/AIDS, or through the use of immunosuppressive medication.

Autoimmunity refers to a collection of autoimmune disorders, in which the organism literally attacks itself. Autoimmune disorders result from hyperactivity of the immune system, wherein the immune system attacks normal tissues as if they were pathogens, physical insults, pathogenic insults or other foreign organisms. Examples of autoimmune diseases are diabetes mellitus type 1, rheumatoid arthritis and systemic lupus erythematosus.

In the human body, the innate immune system recognizes pathogenic insults as well as dead and/or defective cells in the body to initiate protective responses. This recognition occurs through a set of germline-encoded pattern recognition receptors (PRRs) that sense pathogen-associated molecular patterns (PAMPs) and damage-associated molecular patterns (DAMPs).

Cytoplasmic PRRs include NOD-like receptors (NLRs) and retinoic acid-inducible gene-I-like receptors (also known as RIG-I-like receptors). The NLRs recognize ligands from various microbial pathogens, host cells and environmental sources. Based on their domain architecture, NLRs are subdivided into NLRPs and NLRCs. Among these, NLRP1 (mouse NLRP1b), NLRP3 and NLR family apoptosis inhibitory protein/NLCR4 are well established NLRs for their ability to assemble inflammasomes.

Inflammasomes are multimeric cytosolic protein complexes that assemble in response to DAMPs and PAMPs, leading to the activation of inflammatory responses. Inflammasome assembly initiates an inflammatory form of cell death known as pyroptosis, triggering the release of the proinflammatory cytokines interleukin-1β (IL-1β) and IL-18. The NLRP3 inflammasome responds to cellular perturbations and a wide variety of microbes).

The NLRP3 inflammasome is a multimeric cytosolic protein complex that, like other inflammasomes, assembles in response to cellular perturbations. This assembly leads to the activation of caspase-1, which promotes maturation and release of the inflammatory cytokines interleukin-1β (IL-1β) and IL-18, as well as inflammatory cell death (i.e., pyroptosis). The inflammatory cytokines contribute to the development of systemic low-grade inflammation, and aberrant NLRP3 activation can drive a chronic inflammatory state in the body to modulate the pathogenesis of inflammation-associated diseases. Therefore, targeting NLRP3 or other signaling molecules downstream, such as caspase-1, IL-1β or IL-18, has the potential for great therapeutic benefit. However, NLRP3 inflammasome-mediated inflammatory cytokines play dual roles in mediating human disease. While they are detrimental in the pathogenesis of inflammatory and metabolic diseases, they have a beneficial role in numerous infectious diseases and some cancers. Therefore, fine tuning of NLRP3 inflammasome activity is essential for maintaining proper cellular homeostasis and health. The mechanisms of NLRP3 inflammasome activation play divergent roles in the pathogenesis of inflammation-associated diseases such as cancer, atherosclerosis, diabetes and obesity, and therefore offer therapeutic potential when targeted to correct pathways of treatment.

Foam cells, also known as lipid-laden macrophages, are cholesterol-containing cells that can form a plaque and lead to inflammatory maladies such as atherosclerosis which can lead to heart attack and stroke. Foam cells are lipid-rich cells typically with an M2 macrophage-like phenotype, but may exhibit an M1 phenotype, depending on cues. However, certain foam cells may be derived from smooth muscle tissue, and these specific foam cells present a limited macrophage-like phenotype. The presence of the NLRP3 inflammasome is known to promote foam cell formation, which has been observed in several histological samples. However, many studies have also reported that NLRP3 inflammasome activation promotes the formation of macrophage foam cells through IL-1β.

Aluminum hydroxide, denoted by the chemical formula Al(OH)₃ and commonly known in pharmaceutical and related fields as “Alum,” is composed of double layers of hydroxyl groups with aluminum ions occupying two-thirds of the octahedral holes between the double layers. Among several uses, aluminum hydroxide is used as a pharmaceutical adjuvant in some vaccines. Further, aluminum hydroxide is also known to stimulate the immune system by inducing the release of uric acid, which eventually results in stimulation of T cells and B cells.

Recent studies suggest that the nascence and proliferation of cancerous tumors are directly related to inflammatory processes. It has long been known that cancer development and its response to therapy are regulated by inflammation, which either promotes or suppresses tumor progression. Chronic inflammation facilitates tumor progression and treatment resistance, whereas induction of acute inflammatory reactions often stimulates the maturation of dendritic cells (DCs) and antigen presentation, leading to anti-tumor responses.

Conventional cancer treatments are directed at removing or “killing” cancerous tissue to preventing it from spreading. Such treatment options include surgery, chemotherapy, radiation therapy, hormonal therapy, targeted therapy and palliative care. Treatments are usually pursued based on the type, location and grade of the cancer as well as the patient's health and preferences. These options have limitations. They can be ineffective, particularly when cancer has metastasized. Moreover, chemotherapy and radiation therapy have a range of side-effects related to cell toxicity.

Because cancer cells divide faster than most normal cells, they can be sensitive to chemotherapy drugs. However, chemotherapy drugs will also attack other cells in the body, especially fast-dividing cells such as blood cells and the cells lining the mouth, stomach, and intestines, resulting in a narrow therapeutic window. Side effects of the drugs often deter their continued use and have a negative impact on a patient's quality of life. Accordingly, there is a need for improved medications and methods of treating cancer that are more targeted and have less deleterious side effects.

Recent efforts to develop new cancer treatments have focused on immunology. For example, checkpoint therapy can block inhibitory checkpoints restoring immune system function. However, the use of checkpoint inhibitors has limitations and often leads to immunological adverse effects. Altering checkpoint inhibition can have diverse effects on most organ systems of the body. Colitis (i.e., inflammation of the colon) is a common side effect. Infusion of checkpoint inhibitors has also been associated with acute seronegative myasthenia gravis. Moreover, clinical benefits vary and some patients become “hyper-progressors” with accelerated rates of tumor growth and rapid deterioration.

Researchers have also made progress with cancer vaccines. A cancer vaccine is a vaccine that either treats existing cancer or prevents development of cancer. Vaccines that treat existing cancer are known as therapeutic cancer vaccines or tumor antigen vaccines. Some of the vaccines are “autologous,” being prepared from samples taken from the patient, and are specific to that patient. Clinical trials of cancer vaccines in humans, however, have been somewhat disappointing. Although general immune activation directed against the target antigens contained within the cancer vaccine has been documented in most cases, reduction in tumor load has not been frequently observed. Tumor progression and metastasis usually ensue, possibly following a slightly extended period of remission. The failure of cancer vaccines to fulfill their promise is due to the very relationship between host and tumor: through a natural selection process the host leads to the selective enrichment of clones of highly aggressive neoplastically transformed cells, which apparently are so dedifferentiated that they no longer express cancer cell specific molecules. Specific activation of the immune system in such cases only leads to lysis of the remaining cells expressing the particular tumor-associated antigens (TAAs) in the context of the particular human leukocyte antigen (HLA) subclass and the necessary costimulatory molecules. The most dangerous clones of tumor cells however lack these features and thus the cancer vaccine is of little use.

Accordingly, conventional treatments for cancer have limitations and there is a need for improved therapies. Specifically, there is an unmet need for minimally invasive tumor suppression. This need is met through embodiments of the invention as described further throughout the disclosure below.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

The invention relates to methods of treating conditions ameliorated by stimulating an immune response. In aspects, the method includes injection of mesoporous silica rods (MSRs) at or near an affected tissue. In aspects, the MSRs elicit an innate immune response. In aspects, the MSRs stimulate an inflammatory response.

In embodiments, the mesoporous silica rods carry a cytokine payload for injection into a tumor or infection. In aspects, the cytokine payload is time-released or is gradually released (e.g., for 24 hours, two days, five days, etc.).

Accordingly, an embodiment is a mesoporous silica rod that has a cytokine payload for injection into a tumor or infection. The cytokine payload can be interleukin-12 (IL-12) and/or interleukin-2 (IL-2). The mesoporous silica rod can also have an adjuvant (e.g., aluminum hydroxide, a lipopolysaccharide or a toll like receptor agonist) to stimulate an immune response. In aspects, the mesoporous silica rod also has an immune checkpoint inhibitor (e.g., anti-PD1 antibody) or is administered with an immune checkpoint inhibitor.

Another embodiment is a method of treating an ailment. The method can include steps of (a) Identifying an affected tissue and (b) inserting or injecting a mesoporous silica rod at or near the affected tissue. The mesoporous silica rod can carry a cytokine payload and/or an adjuvant. In aspects, the affected tissue is tumor tissue. In aspects, the affected tissue is infected tissue (e.g., bacterial, fungal or viral infection).

Another embodiment is a method of potentiating an endogenous immune response in an affected tissue. The method can be used, for example, to convert a “cold” tumor to a “hot” tumor. The method can include steps of (a) identifying an affected tissue (e.g., a cold tumor) and (b) inserting or injecting a mesoporous silica rod at or near the affected tissue. The mesoporous silica rod can carry a cytokine payload and/or chemokine payload and/or an adjuvant.

Another embodiment is a method of intertumoral delivery of cytokines to a targeted tumor area via injection and implantation of a mesoporous silica rod (MSR) or equivalent mesoporous silica structure or mesoporous silica material to provoke an innate immune response.

In embodiments, the methods described herein further include administration of an anti-PD-1 antibody (or portion of an antibody).

Another embodiment is a method of intertumoral delivery of cytokines to a targeted inflammatory area via injection and/or implantation of a mesoporous silica rod (MSR) or equivalent mesoporous silica structure or mesoporous silica material to provoke an innate immune response.

Another embodiment is a mesoporous silica rod structure carrying a cytokine payload. The mesoporous silica rod can be cylindrical with a plurality of pores. Each pore can have a diameter of about 2 nanometers. In aspects, the pore diameter is about 5 nanometers, 10 nanometers, 15 nanometers, 20 nanometers, 25 nanometers, 30 nanometers, 35 nanometers, 40 nanometers or larger. In aspects, the diameter of the pore varies (e.g., from about 2 nanometers to about 50 nanometers or larger).

In aspects, the cytokine is an interleukin (e.g., IL-2 or IL-12) or multiple interleukins. In aspects, the mesoporous silica rod is cylindrically shaped.

In another embodiment, a mesoporous silica rod structure is coated with a cytokine payload and an adjuvant (e.g., aluminum hydroxide or TLR agonist) for injection and/or implantation into a tumor or other inflammatory area within a subject.

In aspects, the mesoporous silica rod structure delivers a physical insult to a tumor and creates a localized innate immune response which leads to tumor regression.

In aspects, the mesoporous silica rod structure delivers a physical insult to an area that is infected (e.g., by bacteria, fungi or a virus) and creates a localized innate immune response which helps fight the infection.

Other treatment areas targeted by the embodiments above include, for example, sebaceous cysts, acne vulgaris, fatty tumors, abscesses, or any set of aggregated cells that do not form a healthy tissue structure within a human body, organism, or within the integumentary system of a human body or organism.

Another embodiment is a method of stimulating NETosis via injection and/or implantation of a mesoporous silica rod (MSR) or equivalent mesoporous silica structure or mesoporous silica material to provoke.

In aspects, the MSR with IL-12 causes lymphoid aggregates and/or tertiary lymphoid structures.

In aspects, the methods described herein lead to tumor regression and/or an abscopal effect of from a primary tumor. In aspects, the methods increase survival time of a subject suffering from an ailment (e.g., cancer).

Another embodiment is a method of stimulating and/or potentiating an endogenous immune response in an affected tissue. The method can include steps of (a) identifying an affected tissue and (b) inserting mesoporous silica rods at or near the affected tissue.

Another embodiment is a method of converting a “cold” tumor to a “hot tumor.” The method can include steps of (a) identifying a cold tumor in tissue of a subject and (b) inserting mesoporous silica rods at or near the tissue.

Embodiments also include methods of producing and manufacturing mesoporous silica rod structures for modulating an immune response.

Embodiments include a method of producing mesoporous silica rods that includes steps of (a) adding a poloxamer to water to form a solution, (b) mixing the solution, (c) adding an acid, (d) adding a source of silicon dioxide, (e) incubating the solution, (f) sieving and vacuum filtering the solution, and (g) heating the solution to yield the mesoporous silica rods in solution. The final solution can be sterilized. The MSR's are generally stabile in lyophilized form for storage/shipping.

The method can also include a step of mixing the mesoporous silica rods with a granulocyte-macrophage colony-stimulating factor (GM-CSF). The method can also include a step of mixing the mesoporous silica rods with an adjuvant. The method can also include a step of mixing the mesoporous silica rods with a cytosine guanosine dinucleotide (CpG) oligodinucleotide. The method can also include a step of mixing the mesoporous silica rods with a cytokine. The method can also include a step of lyophilizing the mesoporous silica rods.

Other features and advantages of aspects of the present invention will become apparent from the following detailed description, taken in conjunction with the accompanying drawings, which illustrate, by way of example, the principles of aspects of the invention.

### BRIEF DESCRIPTION OF THE DRAWINGS

FIG. 1A depicts a Melanoma Mouse Model CT26 colorectal cancer model for comparing survival times of control and treated mice.

FIG. 1B is a multivariable line graph showing the effect of interleukin-12 (IL-12) as it mediates antitumor activity in a B16F10 melanoma mouse model through stimulation of T, natural killer (NK), and NK T cells through angiogenic effects.

FIG. 2A is a graph showing a survival study of murine models exposed to a 3 μg bolus of IL-12, a 3 μg dose of MSR IL-12 and an untreated control.

FIG. 2B is a graph showing a survival study of murine models exposed to a 6 μg bolus of IL-12, a 6 μg dose of MSR IL-12, and an untreated control.

FIG. 2C is a graph showing a survival study of murine models exposed to a 6 μg bolus of IL-12, a 6 μg dose of MSR IL-12, an MSR intratumoral treatment, and an untreated control.

FIG. 2D is a graph showing a survival study of murine models exposed to a 20 μg bolus of IL-12, a 20 μg dose of MSR IL-12, an MSR intratumoral treatment, and an untreated control.

FIG. 3A is a multivariable line graph showing tumor regression and abscopal effect of treated primary tumor in combined survival studies of FIGS. 2A and 2B.

FIG. 3B is a multivariable line graph showing tumor regression and abscopal effect of untreated contralateral tumor in combined survival studies of FIGS. 2A and 2B.

FIG. 4A is an image of a histological sample of a 20 μg bolus injection of IL-12.

FIG. 4B is an image of a histological sample of a tumor treated with 20 μg of MSR IL-12.

FIG. 4C is an image of a histological sample of an untreated contralateral tumor in a murine model.

FIG. 4D is an image of various histological samples obtained from murine models wherein mesoporous silica rods (MSRs) were injected into tumor sites.

FIG. 5A is a graph showing results of the CT-26 colon carcinoma tumor study (average tumor volume over time). Mice treated with MSR are compared to control (PBS).

FIG. 5B is a graph showing results of the CT-26 colon carcinoma tumor study (average tumor volume over time). Mice treated with MSR are compared to mice treated with IL-12 (6 μg) and MSR+IL-12 (6 μg).

FIG. 5C is a graph showing results of the CT-26 colon carcinoma tumor study (average tumor volume over time). Mice treated with MSR are compared to mice treated with IL-12 (20 μg) and MSR+IL-12 (20 μg).

FIG. 6A is a graph showing results of the CT-26 colon carcinoma tumor study (average tumor volume over time). Mice treated with MSR are compared to control (PBS).

FIG. 6B is a graph showing results of the CT-26 colon carcinoma tumor study (average tumor volume over time). Mice treated with MSR are compared to mice treated with IL-12 (6 μg).

FIG. 6C is a graph showing results of the CT-26 colon carcinoma tumor study (average tumor volume over time). Mice treated with MSR are compared to mice treated with IL-12 (6 μg) and MSR+IL-12 (6 μg).

FIG. 6D is a graph showing results of the CT-26 colon carcinoma tumor study (average tumor volume over time). Mice treated with MSR are compared to mice treated with IL-12 (20 μg).

FIG. 6E is a graph showing results of the CT-26 colon carcinoma tumor study (average tumor volume over time). Mice treated with MSR are compared to mice treated with MSR+IL-12 (20 μg).

FIG. 7A is a graph showing average tumor volume (primary) over time after treatment with (a) PBS, (b) ATT-02 it. p.l., (c) ATT-02+ it. p.l., (d) ATT-02 p.l. 2 flanks and (e) ATT-02+ it. p.l.2 flanks.

FIG. 7B is a graph showing average tumor volume (secondary) over time after treatment with (a) PBS, (b) ATT-02 it. p.l., (c) ATT-02+ it. p.l., (d) ATT-02 p.l. 2 flanks and (e) ATT-02+ it. p.l.2 flanks.

FIG. 8A is a graph showing primary tumor growth (days post inoculation) after treatment with PBS (control).

FIG. 8B is a graph showing primary tumor growth after treatment with ATT-02 it. p.l.

FIG. 8C is a graph showing primary tumor growth after treatment with ATT-02+ it. p.l.

FIG. 8D is a graph showing primary tumor growth after treatment with ATT-02 p.l. flanks.

FIG. 8E is a graph showing tumor growth after treatment with ATT-02+p.l. 2 flanks.

FIG. 9A is a graph showing secondary tumor growth (days post inoculation) after treatment with PBS (control).

FIG. 9B is a graph showing secondary tumor growth after treatment with ATT-02 it. p.l.

FIG. 9C is a graph showing secondary tumor growth after treatment with ATT-02+ it. p.l.

FIG. 9D is a graph showing secondary tumor growth after treatment with ATT-02 p.l. flanks.

FIG. 9E is a graph showing secondary growth after treatment with ATT-02+p.l. 2 flanks.

FIG. 10A is a graph comparing IL-12 levels (pg/mL) over time (hours post-treatment).

FIG. 10B is a graph is a graph comparing IFN gamma levels (pg/mL) over time (hours post-treatment).

FIG. 11A shows the quantification of T- and B-cells at day six after vaccination.

FIG. 11B shows the quantification of T- and B-cells at day twelve after vaccination.

FIG. 11C shows the quantification of macrophages at day six after vaccination.

FIG. 11D show the quantification of monocytes, and neutrophils at day six after vaccination.

FIG. 11E shows the quantification of monocytes, and neutrophils at day twelve after vaccination.

FIG. 12 shows IL-12 detection in mice that received a single subcutaneous injection of 1 mg MSR and 20 μg of IL-12, 5 mg of MSR and 20 μg of IL-12.

FIG. 13A shows white blood cell (WBC) counts versus days post immunization.

FIG. 13B shows lymphocyte (LYM) counts versus days post immunization.

FIG. 13C shows monocyte (MON) counts versus days post immunization.

FIG. 13D shows neutrophil (NEU) counts versus days post immunization.

FIG. 14A shows percent of T-cells in splenocytes (quantification of T-, B-cells) six days post vaccination.

FIG. 14B shows percent of T-cells in splenocytes (quantification of T-, B-cells) fourteen days post vaccination.

FIG. 14C shows percent of macrophages and neutrophils in splenocytes six days post vaccination.

FIG. 14D shows percent of macrophages and neutrophils in splenocytes fourteen days post vaccination.

FIG. 14E shows percent of monocytes and neutrophils in splenocytes six days post vaccination.

FIG. 14F shows percent of monocytes and neutrophils in splenocytes fourteen days post vaccination.

FIG. 15A is a graph showing average primary tumor growth versus time (days post tumor inoculation), single treatment of ATT-02 (1 mg MSR 20 μg IL-12) and ATT-02 cytosine guanosine dinucleotide (CpG) (1 mg MSR 20 μg IL-12) in the primary tumor.

FIG. 15B is a graph showing average secondary tumor growth versus time (days post tumor inoculation).

FIG. 15C is a graph showing primary tumor growth versus time (days post tumor inoculation).

FIG. 15D is a graph showing primary tumor growth versus time (days post tumor inoculation).

FIG. 15E is a graph showing secondary tumor growth versus time (days post tumor inoculation).

FIG. 15F is a graph showing secondary tumor growth versus time (days post tumor inoculation).

FIG. 16A is a graph showing total CD8 T-cells after ATT-02 and ATT-02 CpG treatments.

FIG. 16B is a graph showing total CD8 T-cells after ATT-02 and ATT-02 CpG treatments.

FIG. 16C is a graph showing effector memory T-cells in CD8 T-cells after ATT-02 and ATT-02 CpG treatments.

FIG. 16D shows the results of detection of IFN-γ secreting splenocytes performed by ELISPOT assays.

FIG. 17A is a graph showing average primary volume versus time (days post tumor inoculation) for control, ATT-02 it, ATT-02 pt and ATT-02 μl treatments.

FIG. 17B is a graph showing probability of survival for each group of mice.

FIG. 17C is a graph showing tumor volume versus time (days post tumor inoculation) for control.

FIG. 17D is a graph showing tumor volume versus time (days post tumor inoculation) for ATT-02 i.t.

FIG. 17E is a graph tumor volume versus time (days post tumor inoculation) for ATT-02 p.t.

FIG. 17F is a graph showing tumor volume versus time (days post tumor inoculation) for ATT-02 p.l.

FIG. 18A is a graph showing average primary volume versus time (days post tumor inoculation) for control, ATT-02 it, ATT-02 pt and ATT-02 μl treatments.

FIG. 18B is a graph showing probability of survival for each group of mice.

FIG. 18C is a graph showing tumor volume versus time (days post tumor inoculation) for control.

FIG. 18D is a graph showing tumor volume versus time (days post tumor inoculation) for ATT-02 i.t.

FIG. 18E is a graph tumor volume versus time (days post tumor inoculation) for ATT-02 p.t.

FIG. 18F is a graph showing tumor volume versus time (days post tumor inoculation) for ATT-02 p.l.

FIG. 19A is a graph showing average primary volume versus time (days post tumor inoculation) for double treatments of ATT-02 and ATT-02 CpG intra-tumoral (i.t.), and peri-lymphatic (p.l.) on both flanks in B16F10 melanoma cancer model.

FIG. 19B is a graph showing probability of survival for each group of mice.

FIG. 19C is a graph showing tumor volume versus time (days post tumor inoculation) for control.

FIG. 19D is a graph showing tumor volume versus time (days post tumor inoculation) for ATT-02 i.t. p.l.

FIG. 19E is a graph tumor volume versus time (days post tumor inoculation) for ATT-02 Cpg.

FIG. 19F is a graph showing tumor volume versus time (days post tumor inoculation) for ATT-02 p.l. (2 flanks).

FIG. 19G is a graph tumor volume versus time (days post tumor inoculation) for ATT-02 Cpg. (2 flanks).

FIG. 20 is a graph showing average secondary volume versus time (days post tumor inoculation) for double treatments of ATT-02 and ATT-02 CpG intra-tumoral (i.t.), and peri-lymphatic (p.l.) on both flanks in B16F10 melanoma cancer model.

FIG. 21A is a graph showing tumor volume versus time (days post tumor inoculation) for control.

FIG. 21B is a graph showing tumor volume versus time (days post tumor inoculation) for ATT-02 i.t. p.l.

FIG. 21C is a graph tumor volume versus time (days post tumor inoculation) for ATT-02 Cpg.

FIG. 21D is a graph showing tumor volume versus time (days post tumor inoculation) for ATT-02 p.l. (2 flanks).

FIG. 21E is a graph tumor volume versus time (days post tumor inoculation) for ATT-02 Cpg. (2 flanks).

FIG. 22A shows the quantification of total splenocytes (CD8+ T cells) after MSR, ATT-02 and ATT-02 CpG treatments.

FIG. 22B shows the quantification of total splenocytes (CD8+ T cells and KLRG-1+ and CD127+ effector memory T-cells) after MSR, ATT-02 and ATT-02 CpG treatments.

FIG. 22C shows the quantification of CD127+ effector memory T-cells after MSR, ATT-02 and ATT-02 CpG treatments.

FIG. 22D shows the quantification of macrophages after MSR, ATT-02 and ATT-02 CpG treatments.

FIG. 22E shows the quantification of monocytes after MSR, ATT-02 and ATT-02 CpG treatments.

FIG. 23A shows average tumor volume (mm³) versus time (days post inoculation) after treatment with PBS (control).

FIG. 23B shows average tumor volume (mm³) versus time (days post inoculation) after treatment with IL-12.

FIG. 23C shows average tumor volume (mm³) versus time (days post inoculation) after treatment with PD-1.

FIG. 23D shows tumor volumes (mm³) of treatment group ATT-02.

FIG. 23E shows individual tumor volumes (mm³) of treatment group MSR plus anti PD-1.

FIG. 23F shows tumor volume (mm³) of treatment group ATT-02 plus anti PD-1.

FIG. 24A shows the average tumor volume (mm³) of all treatment groups. Mice were inoculated with 1M CT-26 cells subcutaneously in right flank on day 0. On day 7 mice began treatment with PBS, IL-12 (20 μg, i.t.), PD-1 i.p. every 3 days, ATT-02 (i.t, 20 μg), MSR (i.t.) and PD-1 (i.p) every 3 days, ATT-02 (i.t., 20 μg) plus PD-1 (i.p.) every three days.

FIG. 24B shows the percent change in average weight of treatment groups.

FIG. 25A shows the probability of survival for each group of mice.

FIG. 25B shows average tumor volume (mm³) for naïve, ATT-02 and ATT-02+PD1 treated mice.

FIG. 26A shows average tumor volume (mm³) versus time (days post inoculation) after treatment with PBS (control).

FIG. 26B shows average tumor volume (mm³) versus time (days post inoculation) after treatment with IL-12.

FIG. 26C shows average tumor volume (mm³) versus time (days post inoculation) after treatment with PD-1.

FIG. 26D shows tumor volumes (mm³) of treatment group ATT-02.

FIG. 26E shows individual tumor volumes (mm³) of treatment group MSR plus anti PD-1.

FIG. 26F shows tumor volume (mm³) of treatment group ATT-02 plus anti PD-1.

FIG. 27A shows average tumor volume (mm³) of mice injected with 500K B16F10 cells. Treatment groups include: PBS, IL-12 (20 μg, i.t.), PD-1 i.p. every 3 days, ATT-02 (i.t, 20 μg), MSR (i.t.) and PD-1 (i.p) every 3 days, ATT-02 (i.t., 20 μg) plus PD-1 (i.p.) every 3 days.

FIG. 27B shows the percent change in average weight of treatment groups.

FIG. 28 shows the probability of survival for each group of mice.

FIG. 29A shows tumor volume (mm³) of treatment groups versus time (days post tumor inoculation).

FIG. 29B shows tumor volume (mm³) of treatment groups versus time (days post tumor inoculation).

FIG. 29C shows tumor volume (mm³) of treatment groups versus time (days post tumor inoculation).

FIG. 30A shows average tumor volume (mm³) versus time (days post inoculation) after treatment with PBS (control).

FIG. 30B shows average tumor volume (mm³) versus time (days post inoculation) after treatment with IL-12.

FIG. 30C shows average tumor volume (mm³) versus time (days post inoculation) after treatment with PD-1.

FIG. 30D shows tumor volumes (mm³) of treatment group ATT-02.

FIG. 30E shows individual tumor volumes (mm³) of treatment group MSR plus anti PD-1.

FIG. 30F shows tumor volume (mm³) of treatment group ATT-02 plus anti PD-1.

FIG. 31A shows average tumor volume (mm³) versus time (days post inoculation) after treatment with PBS (control).

FIG. 31B shows average tumor volume (mm³) versus time (days post inoculation) after treatment with IL-12.

FIG. 31C shows average tumor volume (mm³) versus time (days post inoculation) after treatment with PD-1.

FIG. 31D shows tumor volumes (mm³) of treatment group ATT-02.

FIG. 31E shows individual tumor volumes (mm³) of treatment group MSR plus anti PD-1.

FIG. 31F shows tumor volume (mm³) of treatment group ATT-02 plus anti PD-1.

FIG. 32A shows average tumor volume (mm³) versus time (days post inoculation). The PBS treated mice showing primary (p) versus secondary (c) tumor volumes assessing abscopal effect in 2 out of 5 mice.

FIG. 32B shows average tumor volume (mm³) versus time (days post inoculation) after treatment with IL-12. The MSR treated mice showed primary (p) versus secondary (c) tumor volumes assessing abscopal effect in 3 out of 5 mice.

FIG. 32C shows average tumor volume (mm³) versus time (days post inoculation) after treatment with PD-1. The IL-12 treated mice showing primary (p) versus secondary (c) tumor volumes assessing abscopal effect in 2 out of 5 mice.

FIG. 32D shows average tumor volume (mm³) versus time (days post inoculation) after treatment with PD-1. The ATT-02 treated mice showed primary (p) versus secondary (c) tumor volumes assessing abscopal effect in 4 out of 5 mice.

FIG. 33 shows the probability of survival for each group of mice.

FIG. 34A shows the tumor volumes (mm³) of treatment groups PBS control compared to IL-12 i.t.

FIG. 34B shows the tumor volumes (mm³) of PBS control compared to ATT-02 treatment.

FIG. 34C shows the tumor volumes (mm³) of treatment ATT-02 compared to IL-12.

FIG. 34D shows the percent change in mice body weight over the course of the study.

FIG. 35 shows average tumor volumes (mm³) versus time (days post tumor inoculation) for the treatment groups.

FIG. 36 shows the probability of survival for each group of mice.

FIG. 37A shows the average primary tumor burden volumes (mm³) of treatment groups in the single dose mice.

FIG. 37B shows the tumor volumes (mm³) of the primary right-flank of the PBS control.

FIG. 37C shows the tumor volumes (mm³) of the primary right-flank of the ATT-02 low treatment.

FIG. 37D shows the tumor volumes (mm³) of the primary right-flank of the ATT-02 high treatment.

FIG. 37E shows the average secondary tumor burden volumes (mm³) of treatment groups in the single dose mice.

FIG. 37F shows the tumor volumes (mm³) of the secondary left-flank of the PBS control.

FIG. 37G shows the tumor volumes (mm³) of the secondary left-flank of the ATT-02 low treatment.

FIG. 37H shows the tumor volumes (mm³) of the secondary left-flank of the ATT-02 high treatment.

FIG. 38A shows the average primary tumor burden volumes (mm³) of treatment groups in the single dose mice.

FIG. 38B shows the tumor volumes (mm³) of the primary right-flank of the PBS control multi-dose.

FIG. 38C shows the tumor volumes (mm³) of the primary right-flank of the ATT-02 low treatment multi-dose.

FIG. 38D shows the tumor volumes (mm³) of the primary right-flank of the ATT-02 high treatment multi-dose.

FIG. 38E shows the average secondary tumor burden volumes (mm³) of treatment groups in the single dose mice multi-dose.

FIG. 38F shows the tumor volumes (mm³) of the secondary left-flank of the ATT-02 high multi-dose treatment.

FIG. 39 shows the probability of survival for each group of mice.

FIG. 40A shows AT055 Att-02 Therapeutic Effects After Single Dose in B16F10 Model (day 7 total CD8+ T-cells, KLRG1 and CD127+ cells).

FIG. 40B shows AT055 Att-02 Therapeutic Effects After Single Dose in B16F10 Model (day 7 total CD8+ T-cells, KLRG1 and CD127+ cells).

FIG. 40C shows AT055 Att-02 Therapeutic Effects After Single Dose in B16F10 Model (day 14 total CD8+ T-cells, KLRG1 and CD127+ cells).

FIG. 40D shows AT055 Att-02 Therapeutic Effects After Single Dose in B16F10 Model (day 14 total CD8+ T-cells, KLRG1 and CD127+ cells).

FIG. 41 shows the average primary tumor burden volumes (mm³) of the treatment groups (with PBS or ATT-02+/−CpG either perilymphatic (p.l) or intratumoral (i.t)).

FIG. 42A shows the average primary tumor burden volumes (mm³) of treatment group PBS control.

FIG. 42B shows the tumor volumes (mm³) of MSR perilymphatic treatment.

FIG. 42C shows the tumor volumes (mm³) of ATT-02 i.t treatment.

FIG. 42D shows the tumor volumes (mm³) of treatment group ATT-02 perilymphatic.

FIG. 42E shows the tumor volumes (mm³) of treatment group ATT-02 plus CpG i.t.

FIG. 42F shows the tumor volumes (mm³) of treatment group ATT-02 plus CpG perilymphatic.

FIG. 43 shows the average secondary tumor burden volumes (mm³) of the treatment groups.

FIG. 44A shows the average primary tumor burden volumes (mm³) of treatment group PBS control.

FIG. 44B shows the tumor volumes (mm³) of MSR perilymphatic treatment.

FIG. 44C shows the tumor volumes (mm³) of ATT-02 i.t treatment.

FIG. 44D shows the tumor volumes (mm³) of treatment group ATT-02 perilymphatic.

FIG. 44E shows the tumor volumes (mm³) of treatment group ATT-02 plus CpG i.t.

FIG. 44F shows the tumor volumes (mm³) of treatment group ATT-02 plus CpG perilymphatic.

FIG. 45 shows the probability of survival for each group of mice.

FIG. 46A shows total CD8+ T cells population (AT063: CD8+ T-cell Population after Treatment).

FIG. 46B is CD8+ TCF-1+ population.

FIG. 46C is CD8+ KLRG1+ population.

FIG. 46D is the CD8+CD127+ population.

FIG. 46E is the CD8+CD44+ population.

FIG. 47A shows total CD11b+GR-1+ population (AT063: CD11b+ Population after Treatment).

FIG. 47B shows CD11b+CD86+ population.

FIG. 47C shows CD11b+MHCII+ population.

FIG. 48A shows total CD11c+GR-1+ population.

FIG. 48B shows CD11c+CD86+ population.

FIG. 48C shows CD11c+MHCII+ population.

FIG. 49 shows the IFN gamma expression by treatment groups (AT063 Att-02 Therapeutic Effects After Single Dose in B16F10 Model).

FIG. 50 depicts an overview of mesoporous silica rod manufacture. A symmetric triblock copolymer (Pluronic P123) composed of poly(ethylene oxide) and poly(propylene oxide) is employed to create form rod shaped micelles in solution. Tetraethyl Orthosilicate (TEOs) is added to the solution and the silica deposits on the micelles, creating a hexagonal pore structure. The Pluronic P123 is rinsed and calcinated (high temperature treatment) to remove the polymer, leaving a silica mesoporous structure.

FIG. 51 is a process flowchart for synthesizing mesoporous silica rods (MSRs).

FIG. 52A is a representative SEM sizing image of standard MSR width.

FIG. 52B is a representative SEM sizing image of standard MSR length.

FIG. 53 is a process flowchart of a proposed manufacturing process of mesoporous silica rods (MSRs).

FIG. 54A-54D show representative SEM sizing images of MSR length and width. Modified MSRs length (FIG. 54A) and width (FIG. 54B). Standard MSRs length (FIG. 54C) and width (FIG. 54D). D[n,0.1] (μm), D[n,0.5] (μm) D[n,0.9] (μm)=biodistribution of MSRs sized at 10% or less, 50% or less, and 90% or less respectively within the total number mean.

FIG. 55 depicts the IL-12 in situ vaccine paradigm.

FIG. 56A shows the average primary tumor burden volumes (mm³) versus time (days post tumor inoculation) for different treatment groups (P=0.01, IL-12 vs. ATT-02, 17 days).

FIG. 56B shows the probability of survival for each group of mice (P=0.01, IL-12 vs. ATT-02, log-rank).

FIG. 56C shows the average primary tumor burden volumes (mm³) versus time (days post tumor inoculation) for different treatment groups.

FIG. 56D shows the probability of survival for each group of mice (P=0.02, IL-12 vs. ATT-02, log-rank).

FIG. 56E shows the average primary tumor burden volumes (mm³) versus time (days post tumor inoculation) for different treatment groups (P=0.01, IL-12 vs. ATT-02, 20 days).

FIG. 56F shows the probability of survival for each group of mice.

FIG. 57A shows the average primary tumor burden volumes (mm³) versus time (days post tumor inoculation) for different treatment groups (P<0.01, PBS vs. ATT-02 low, 21 days; P<0.01, PBS vs. ATT-02 high, 21 days).

FIG. 57B shows the average secondary tumor burden volumes (mm³) versus time (days post tumor inoculation) for different treatment groups (P<0.01, PBS vs. ATT-02 low, 21 days; P<0.01, PBS vs. ATT-02 high, 21 days).

FIG. 57C shows the probability of survival for each group of mice (ATT-02 low dose 1× vs. ATT-02 3×, p=0.002, log-rank).

FIG. 57D shows the fold change in gene expression compared to untreated controls (primary/untreated).

FIG. 57E shows the fold change in gene expression compared to untreated controls (secondary/treated).

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “mesoporous” generally refers to a material having pores of a size between 1 and 50 nanometers (nm).

The term “mesoporous silica” refers to a form of silica that is characterized by its mesoporous structure (i.e., having pores that range from 1 nm to 50 nm in diameter). Mesoporous silica is a relatively recent development in nanotechnology. The most common types of mesoporous nanoparticles are MCM-41 and SBA-15. Research continues on the particles, which have applications in catalysis, drug delivery and imaging. Mesoporous ordered silica films have been also obtained with different pore topologies.

The term “mesoporous silica rods” or “MSRs” refers to a nanoparticle composed of mesoporous silica that is substantially rod-shaped (i.e., a straight substantially cylindrical structure that is longer than it is wide). MSRs have also been used for sensing and interparticle communication protocols. Their large surface area and high loading capacity makes them suitable for the delivery of a variety of drugs, antibodies, genes, proteins and peptides. They can serve as vaccine adjuvants; vaccine vehicles for cancer antigens and pathogen-associated molecular patterns delivery to APC; tools for T-cell priming, expanding, trafficking, infiltration, and recognition of cancer cells; agents that cause the release of tumor antigens by photodynamic and photothermal therapies; chemotherapy agents that cause the direct killing of malignant cells or inhibit the immune checkpoint; agents for starvation therapies or combinations thereof.

The term “MCM-41” or “Mobil Composition of Matter No. 41” refers to a mesoporous material with a hierarchical structure from a family of silicate and alumosilicate solids. MCM-41 consists of a regular arrangement of cylindrical mesopores that form a one-dimensional pore system. It is characterized by an independently adjustable pore diameter, a sharp pore distribution, a large surface and a large pore volume. The pores are larger than with zeolites and the pore distribution can easily be adjusted. The mesopores have a diameter of 2 nm to 6.5 nm.

The term “SBA-15” or “Santa Barbara Amorphous-15” refers to a stable mesoporous silica sieve that has high hydrothermal and mechanical stability from a framework of uniform hexagonal pores that feature a narrow pore-size distribution and a tunable pore diameter (i.e., 5 nm to 15 nm) but, most significantly, from its relatively thick walls, which range between 3.1 nm and 6.4 nm. SBA mesoporous silica 15 has a high internal surface area, which lends itself to various applications, including environmental adsorption and separation, advanced optics and catalysts.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

A “pharmaceutical composition” can include the combination of an active agent, such as a therapeutic peptide, with a carrier, inert or active, in a sterile composition suitable for diagnostic or therapeutic use in vitro, in vivo or ex vivo.

As used herein, the term “prevention” means all of the actions by which the occurrence of the disease is restrained or retarded.

The term “treating” or “treatment” refers to one or more of (1) inhibiting the disease (i.e., arresting further development of the pathology and/or symptomatology); and (2) ameliorating the disease (i.e., reversing the pathology and/or symptomatology) such as decreasing the severity of disease.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration.

The term “inflammation” refers to part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. The five cardinal signs are heat, pain, redness, swelling, and loss of function. Inflammation is a generic response, and therefore it is considered as a mechanism of innate immunity, as compared to adaptive immunity, which is specific for each pathogen. Too little inflammation could lead to progressive tissue destruction by the harmful stimulus (e.g., bacteria) and compromise the survival of the organism. In contrast, too much inflammation, in the form of chronic inflammation, is associated with various diseases, such as hay fever, periodontal disease, atherosclerosis, and osteoarthritis. Inflammation can be classified as either acute or chronic. Acute inflammation is the initial response of the body to harmful stimuli and is achieved by the increased movement of plasma and leukocytes (in particular granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process.

The term “inflammatory disorder” or “inflammation condition” refers to a condition in which the immune system mistakenly attacks one's own cells or tissues. This causes abnormal inflammation that can result in chronic pain, redness, swelling, stiffness and damage to otherwise healthy body tissues. Inflammatory conditions can affect the nervous system (e.g., encephalitis, myelitis, meningitis, arachnoiditis and neuritis). Inflammatory conditions can affect the eyes (e.g., dacryoadenitis, scleritis, episcleritis, keratitis, retinitis, chorioretinitis, blepharitis, conjunctivitis and uveitis). Inflammatory conditions can affect the ears (e.g., Otitis externa, Otitis media, Labyrinthitis and Mastoiditis). Inflammatory conditions can affect the cardiovascular system (e.g., Endocarditis, Myocarditis, Pericarditis, Arteritis, Phlebitis and Capillaritis). Inflammatory conditions can affect the respiratory system (e.g., Sinusitis, Rhinitis Pharyngitis, Laryngitis, Tracheitis, Bronchitis, Bronchiolitis, Pneumonitis, Pleuritis and Mediastinitis. Inflammatory conditions can affect the mouth and digestive system (e.g., Stomatitis, Gingivitis, Gingivostomatitis, Glossitis, Tonsillitis, Sialadenitis/Parotitis, Cheilitis, Pulpitis, Gnathitis, Esophagitis, Gastritis, Gastroenteritis, Enteritis, Colitis, Enterocolitis, Duodenitis, Ileitis, Caecitis, Appendicitis and Proctitis). Inflammatory conditions can affect the accessory digestive organs (e.g., Hepatitis, Ascending cholangitis, Cholecystitis, Pancreatitis and Peritonitis). Inflammatory conditions can affect the integumentary system (e.g., Dermatitis, Folliculitis, Cellulitis and Hidradenitis). Inflammatory conditions can affect the musculoskeletal system (e.g., Arthritis Dermatomyositis, Myositis, Synovitis/Tenosynovitis, Bursitis, Enthesitis, Fasciitis, Capsulitis, Epicondylitis, Tendinitis, Panniculitis, Osteochondritis: Osteitis/Osteomyelitis, Spondylitis, Periostitis and Chondritis). Inflammatory conditions can affect the urinary system (e.g., Nephritis, Glomerulonephritis, Pyelonephritis, Ureteritis, Cystitis and Urethritis). Inflammatory conditions can affect the female reproductive system (e.g., Oophoritis, Salpingitis, Endometritis, Parametritis, Cervicitis, Vaginitis, Vulvitis and Mastitis). Inflammatory conditions can affect the male reproductive system (e.g., Orchitis, Epididymitis, Prostatitis, Seminal vesiculitis, Balanitis, Posthitis and Balanoposthitis. Inflammatory conditions can affect the endocrine system (e.g., Insulitis, Hypophysitis, Thyroiditis, Parathyroiditis and Adrenalitis). Inflammatory conditions can also affect the lymphatic system (e.g., Lymphangitis and Lymphadenitis).

The term “autoimmune disease” or “autoimmune disorder” refers to a condition arising from an abnormal immune response to a functioning body part. Common autoimmune diseases include Addison disease, Celiac disease, Dermatomyositis, Graves disease, Hashimoto thyroiditis, Multiple sclerosis, Myasthenia gravis and Pernicious anemia.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

The term “potentiating an endogenous immune response” refers to increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

The term “neoplasia” refers to a disease that is caused by or results in inappropriately high levels of cell division, inappropriately low levels of apoptosis, or both. For example, cancer is an example of a neoplasia. Examples of cancers include, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “cancer” refers to human cancers and carcinomas, sarcomas, adenocarcinomas, etc., including solid tumors, kidney, breast, lung, kidney, bladder, urinary tract, urethra, penis, vulva, vagina, cervical, colon, ovarian, prostate, pancreas, stomach, brain, head and neck, skin, uterine, testicular, esophagus, and liver cancer. In any of the embodiments above, one or more cancer therapies, e.g., chemotherapy, radiation therapy, immunotherapy, surgery, or hormone therapy can be co-administered further with the methods described herein.

The term “abscopal effect” refers to a hypothesis in the treatment of metastatic cancer whereby shrinkage of untreated tumors occurs concurrently with shrinkage of tumors within the scope of the localized treatment. It is thought that in the abscopal effect, the immune system is stimulated to fight cancer in the whole body as a result of the local therapy.

The term “infectious disease” refers to bacterial, protozoan, and viral pathogens that infect humans and cause disease. Viral pathogens include, for example, human immunodeficiency virus, hepatitis B virus, hepatitis C virus, herpes virus. Bacterial and protozoal pathogens can include E. coli, Staphylococcus sp., Streptococcus sp., Mycobacterium tuberculosis, Giardia, Malaria, Leishmania, and Pseudomonas aeruginosa. An infectious pathogen can be a capable of establishing chronic infections (e.g., those that are prolonged or persistent).

The term “skin and soft tissue infections” or “SSTIs” encompass any type of microorganism (i.e., bacterial, viral or fungal) that enters any break in the skin and can invade the subcutaneous tissue (soft tissue under the skin), fascia (connective tissue), and muscles.

The term “cytokine” or “cytokines” generally refers to any of various small regulatory proteins that regulate the cells of the immune system. Examples include substances, such as interferon, interleukin, and growth factors, which are secreted by certain cells of the immune system and have an effect on other cells.

The term “chemokine” refers to any of a class of cytokines with functions that include attracting white blood cells to sites of infection. Chemokines are a family of small cytokines or signaling proteins secreted by cells that induce directional movement of leukocytes, as well as other cell types, including endothelial and epithelial cells. In addition to playing a major role in the activation of host immune responses, chemokines are important for biological processes, including morphogenesis and wound healing, as well as in the pathogenesis of diseases like cancers.

The term “intratumorally” generally refers to “within a tumor.”

The term “intertumorally” generally refers to “between tumors.”

The term “physical insult” generally refers to the action or cause of any kind of injury, disturbance, or disruption to an organism's body, including but not limited to any action or cause of any kind of injury, disturbance, or disruption to an organism's tissues.

The term “inflammasome” generally refers to a multi-protein complex that is responsible for inflammatory rheumatic diseases via activation of caspases.

The term “pyroptosis” generally refers to a form of programmed cell death associated with antimicrobial responses during inflammation.

The term “immunogenic cell death” generally refers to any type of cell death eliciting an immune response.

The term “neutrophil” refers to a type of white blood cell, also known as neutrocytes or heterophils, that form an essential part of the innate immune system.

The term “macrophage” refers to a white blood cell that phagocytize necrotic cell debris and foreign material, including viruses, bacteria and tattoo ink.

The term “dendritic cells” refers to any cells that have branching processes and which form a part of the mammalian immune system.

The term “Interleukin/IL” generally refers to any of a group of cytokine proteins important in the regulation of lymphocyte protein.

The term “Interleukin-12/IL-12” generally refers to an effective antitumoral cytokine belonging to the familial group of cytokine proteins.

The term “bolus injection” generally refers to a single dose of a drug or other medicinal preparation that is administered all at once.

The term “adjuvant” generally refers to a substance that increases or modulates the immune response to a vaccine. An adjuvant can help create a stronger immune response in people receiving a vaccine. Common adjuvants include Aluminum, AS01B, AS04, CpG 1018, MatrixM™ and MF59.

The term “adjuvant therapy” or “neoadjuvant therapy” refers to using adjuvants in combination with a primary treatment (e.g., surgery or radiation) to decrease the chance of the cancer returning. It is often used to make the primary treatment more effective.

The term “NLRP3 inflammasome” refers to a critical component of the innate immune system that mediates caspase-1 activation and the secretion of proinflammatory cytokines IL-1β/IL-18 in response to microbial infection and cellular damage. However, the aberrant activation of the NLRP3 inflammasome has been linked with several inflammatory disorders, which include cryopyrin-associated periodic syndromes, Alzheimer's disease, diabetes, and atherosclerosis. The NLRP3 inflammasome is activated by diverse stimuli, and multiple molecular and cellular events, including ionic flux, mitochondrial dysfunction, and the production of reactive oxygen species. Lysosomal damage has been shown to trigger its activation.

The term “CCL2,” “chemokine (C—C motif) ligand 2” or “monocyte chemoattractant protein 1 (MCP1)” or “small inducible cytokine A2” refers to a small cytokine that belongs to the CC chemokine family. CCL2 recruits monocytes, memory T cells, and dendritic cells to the sites of inflammation produced by either tissue injury or infection. CCL2 is implicated in pathogeneses of several diseases characterized by monocytic infiltrates, such as psoriasis, rheumatoid arthritis and atherosclerosis.

The term “PD-1,” “Programmed cell death protein 1” or “CD279” refers to a protein on the surface of cells that has a role in regulating the immune system's response to the cells of the human body by down-regulating the immune system and promoting self-tolerance by suppressing T cell inflammatory activity. Engagement of PD-1 by either of its ligands, PD-L1 or PD-L2, on an adjacent cell inhibits TCR signaling and TCR-mediated proliferation, transcriptional activation and cytokine production. This prevents autoimmune diseases, but it can also prevent the immune system from killing cancer cells. Therapeutic antibodies designed to block the PD-1/PD-L1 interaction have potential for the treatment of cancer. PD-L1 binds to its receptor, PD-1, found on activated T cells, B cells, and myeloid cells, to modulate activation or inhibition. Several inhibitors of programmed cell death-1 (PD-1) and programmed death ligand-1 (PD-L1) have been approved as a form of immunotherapy for multiple cancers. Examples include Pembrolizumab (Keytruda), Nivolumab (Opdivo) and Cemiplimab (Libtayo).

As used herein, the term “antibody” refers to a polypeptide or a polypeptide complex that specifically recognizes and binds to an antigen through one or more immunoglobulin variable regions. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding and is encoded by the variable domain. An antibody can be a whole antibody, an antigen binding fragment or a single chain thereof.

The term “agonist antibody” refers to an antibody that stimulates or activates an organ. An antibody can act as an agonist of a receptor, essentially replacing the activity of the normal ligand. The agonist activity can occur when the antibody binds the receptor in a manner that mimics the binding of the physiological ligand resulting in antibody-mediated agonism. For example, agonistic antibodies against the thyrotropin receptor in Grave's disease stimulate the thyroid gland to release thyroid hormones that produce hyperthyroidism. Agonistic antibodies may also stimulate when clustered, either via the Fc portion of the antibody engaging an Fc receptor in trans or cis, or through antigen mediated clustering. The latter clustering mechanism requires antigen engagement by one half of a bispecific molecule and engagement of the stimulatory receptor by the second half of a bispecific molecule. Exemplary stimulatory receptors are CD3, CD28 and 4-1BB, which stimulate T cells.

The terms “antibody fragment” or “antigen-binding fragment” are used with reference to a portion of an antibody, such as Fab′, Fab, Fv, scFv and the like. Regardless of structure, an antibody fragment binds with the same antigen that is recognized by the intact antibody. The term “antibody fragment” also includes diabodies and any synthetic or genetically engineered proteins comprising immunoglobulin variable regions that act like an antibody by binding to a specific antigen to form a complex.

The term “immunogenicity” refers to the ability of cells/tissues to provoke an immune response. It is generally considered to be an undesirable physiological response.

The term “immunogenic tumor” refers to a tumor with sufficient antigens and priming that elicits good T cell responses in the tumor draining lymph node. In contrast, poorly immunogenic tumors fail to generate T cell responses. The ability of tumors to respond to T cell control is not necessarily linked to their ability to prime T cell responses.

The term “immunogenic cell death,” “ICD” or “immunogenic apoptosis” refers to a form of cell death resulting in a regulated activation of the immune response. This cell death is characterized by apoptotic morphology, maintaining membrane integrity. Endoplasmic reticulum (ER) stress is generally recognized as a causative agent for ICD, with high production of reactive oxygen species (ROS). Two groups of ICD inducers are recognized. Type I inducers cause stress to the ER only as collateral damage, mainly targeting DNA or chromatin maintenance apparatus or membrane components. Type II inducers target the ER specifically. ICD is induced by some cytostatic agents such as anthracyclines, oxaliplatin and bortezomib, or radiotherapy and photodynamic therapy (PDT). Some viruses can be listed among biological causes of ICD. Just as immunogenic death of infected cells induces immune response to the infectious agent, immunogenic death of cancer cells can induce an effective antitumor immune response through activation of dendritic cells (DCs) and consequent activation of specific T cell response. This effect is used in antitumor therapy.

The term “immune checkpoint” or “checkpoint” refers to a regulator of the immune system. Immune checkpoints are crucial for self-tolerance, which prevents the immune system from attacking cells indiscriminately. However, some cancers can protect themselves from attack by stimulating immune checkpoint targets. Checkpoint inhibitor therapy is a form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function.

Tumor types can be categorized into separate groups based on their response rate to immune checkpoint inhibitors (ICI). In practical terms, which can be understood more clearly in the medical field, tumor types are categorized as either “hot” or “cold.” Hot tumors have a high mutation load and respond faster to ICIs. They accumulate a lot of mutations, which causes tumor cells to produce specific molecules, neoantigens, on their cell surface. These neoantigens make the tumor more vulnerable to recognition by the immune system and thus more likely to elicit a strong immune response. Malignant tumors considered “hot” include cancer of the bladder, head and neck, kidney cancer, liver cancer, melanoma, and non-small cell lung cancer, as well as tumors of various types with a high rate of microsatellite instability. It is in these types of tumors that inhibitors of immune checkpoints are effective. Cold tumors have a low response rate and are often compared to an impregnable fortress surrounded by a moat. There are few T-cells in their “walls” and it is difficult for them to mobilize an immune response. Common cancers that have “cold” tumors include glioblastoma, ovarian, prostate, and pancreatic cancer.

The term “administration” refers to the introduction of an amount of a predetermined substance into a patient by a certain suitable method. The composition disclosed herein may be administered via any of the common routes, as long as it is able to reach a desired tissue, for example, but is not limited to, intraperitoneal, intravenous, intramuscular, subcutaneous, intradermal, oral, topical, intranasal, intrapulmonary, or intrarectal administration.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13th Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Immunotherapy refers to a type of cancer treatment that uses substances made by the body to boost the immune system and help the body find and destroy cancer cells. Immunotherapy has emerged as a powerful useful approach for cancer treatment in recent years. Some of its advantages over traditional cancer therapies (i.e., chemotherapy, radiotherapy, and surgery) are the significant improvement in patients' quality of life and survival percentage. Immunotherapy can be used to treat many different types of cancer and can be used in combination with chemotherapy and/or other cancer treatments.

The present invention is thus based on the surprising discovery that injection of mesoporous silica rods (MSRs) stimulates immune activity to high levels in a targeted tissue. MSRs can trigger cancer immunity cycle activation while avoiding harming healthy cells. Thus, the MSRs can be used as a scaffold to deliver cytokines intratumorally. In embodiments, the methods described herein are suitable for treatment of a condition known or expected to be ameliorated by immune stimulation (e.g., cancer or infection).

### Mesoporous Silica Rods (MSRs)

Mesoporous silica is a form of silica that is characterized by its mesoporous structure, (i.e., having pores that range from 1 nm to 50 nm in diameter). Mesoporousity is generally defined between microporous (i.e., <2 nm) and macroporous (i.e., >50 nm). Mesoporous silica is a relatively recent development in nanotechnology. Mesoporous ordered silica films have been also obtained with different pore topologies.

Mesoporous silica rods (MSRs) can be injected into a patient rather than surgically implanted like other scaffolds. MSRs can assemble into 3D microenvironments for dendritic cells directly in the body. In some embodiments, long rod-like microparticles that are orders of magnitude larger than the size of one immune cell are used. In embodiments, these microparticles are injected into tissues (e.g., with a standard 23-gauge syringe). Due to their size, they do not diffuse away from the injection site.

The large surface area of the pores allows the particles to be filled with a drug or a cytotoxin. Like a Trojan Horse, the particles can either be detected through receptors or taken up by certain biological cells through endocytosis, depending on what chemicals are attached to the outside of the particles.

Mesoporous silica can also boost the in vitro and in vivo dissolution of poorly water-soluble drugs. Many drug-candidates coming from drug discovery suffer from poor water solubility. Such drugs can be administered with MSRs offer a promising mechanism for drug delivery.

### Cytokines

Cytokines are a broad and loose category of small proteins (e.g., 5-25 kDa) that are important in cell signaling. Cytokines also are peptides and cannot cross the lipid bilayer of cells to enter the cytoplasm. Cytokines have been shown to be involved in autocrine, paracrine and endocrine signaling as immunomodulating agents.

Cytokines include chemokines, interferons, interleukins, lymphokines, and tumor necrosis factors, but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell. They act through cell surface receptors and are especially important in the immune system; cytokines modulate the balance between humoral and cell-based immune responses, and they regulate the maturation, growth, and responsiveness of particular cell populations. Some cytokines enhance or inhibit the action of other cytokines in complex ways. Cytokines are important in health and disease, specifically in host immune responses to infection, inflammation, trauma, sepsis, cancer, and reproduction.

Interleukin-12 (IL-12) is an interleukin that is naturally produced by dendritic cells, macrophages, neutrophils, and human B-lymphoblastoid cells (NC-37) in response to antigenic stimulation. IL-12 belongs to the family of interleukin-12. IL-12 family is unique in comprising the only heterodimeric cytokines, which includes IL-12, IL-23, IL-27 and IL-35. IL-12 also has anti-angiogenic activity (i.e., it can block the formation of new blood vessels) thus can be used as an antineoplastic agent. Despite sharing many structural features and molecular partners, they mediate surprisingly diverse functional effects.

Interleukin-2 (IL-2) is an interleukin, a type of cytokine signaling molecule in the immune system. It regulates the activities of white blood cells (leukocytes, often lymphocytes) that are responsible for immunity. IL-2 is part of the body's natural response to microbial infection, and in discriminating between foreign (“non-self”) and “self.” IL-2 mediates its effects by binding to IL-2 receptors, which are expressed by lymphocytes. The major sources of IL-2 are activated CD4+ T cells and activated CD8+ T cells.

Applicants have discovered surprising results utilizing mesoporous silica rods (MSRs) that are configured as scaffolds to deliver cytokines intratumorally. The combination of the MSRs and cytokines modulate the immune cell population and activation states in a manner that can alter the tumor microenvironment. This leads to a more effective response by the immune system and improved survival rates in cancer studies.

Without being bound by theory, Applicants propose that the MSRs can trigger multiple pathways of the immune system. MSRs can increase the probability of high endothelial venules (HEVs) forming in a tumor microenvironment. HEVs detected in tumors is associated with better response to immune checkpoint inhibitors in the clinic. MSRs can also recruit neutorophils, lymphocytes, dendritic cells, and macrophages (including foam cells) to the injection site. Through reducing the toxicity of cytokines by controlling and localizing their release, the invention serves to change the therapeutic window of cytokines such as IL-2 and IL-12, by exposing tumors and areas of inflammation to a locally high concentration in combination with inflammatory signals induced by implanted and/or injected silica materials. In embodiments, the agents (e.g., cytokines) and MSRs work synergistically with one another.

The treatment of subjects with mesoporous silica rods can trigger an enhanced immune response through a physical insult. Through this mechanism, numerous populations of immune cells are recruited to the targeted area of treatment, influencing and activating innate immune pathways. The controlled chemistry and configuration of MSRs and other suitable silica materials activate specific immune pathways in combination with cytokines, IL-12, IL-2, inflammasomes and other regulatory proteins. Immune cells organize around the MSRs.

The methods described herein provide several advantages over conventional treatments for cancer/inflammation. The MSR delivery system can be modified or configured to:
a) release a variety of cargos (e.g., cytokines) at different rates,
b) effectively deliver large cargo utilizing the high surface area of the MSRs,
c) increase the half-life of target cytokines,
d) control the exposure and concentration of cytokine release, and
e) suspend cargos and safely release them over time (e.g., from days to months).

One or more additional agents can be included on the MSRs. In embodiments, adjuvants are included on the MSRs. In embodiments, the MSRs described herein can be targeted to a specific tissue such as a tumor. The MSRs can also be used to treat an ailment related to infection and/or inflammation.

The treatments described herein allow for the creation of an alternative immunological environment within the tumor and/or inflammatory site which creates responses that improve tumor and inflammatory regression. The material composition of the mesoporous silica structures further allows for immunogenic cell death (ICD) which creates antigen sources for further signaling immune cell proliferation, therefore focusing agents such as neutrophils, macrophages, and dendritic cells to targeted treatment areas for preferable outcomes such as tumor suppression, tumor regression, tumor-size reduction, tumor elimination, inflammatory reduction, inflammatory suppression, inflammatory regression and elimination of inflammation within targeted areas.

While the examples below describe the use of interleukin-12 (IL-12) and/or interleukin-2 (IL-2), the invention can be used with other cytokines, including IL-1, IL-2, IL-3, GM-CSF, IL-4, IL-5, IL-6, IL-7, IL-9, IL-10, IL-11, IL-12, IL-13, IL15, IL-21, IL-28, TNF-alpha, IL-23, IL-16, IL-17, TNF-α, TNF-β, interferons, IL-10, IL-19, IL-20, IL-22 and IL-24

### Adjuvants

An adjuvant can be defined as a vaccine component that enhances the magnitude, breadth and/or durability of an immune response. Due to the variety of mechanisms and links between the innate and adaptive immune response, an adjuvant-enhanced innate immune response results in an enhanced adaptive immune response. Specifically, adjuvants may exert their immune-enhancing effects according to five immune-functional activities.

First, adjuvants may help in the translocation of antigens to the lymph nodes where they can be recognized by T cells. This will ultimately lead to greater T cell activity resulting in a heightened clearance of pathogen throughout the organism. Second, adjuvants may provide physical protection to antigens which grants the antigen a prolonged delivery. This means the organism will be exposed to the antigen for a longer duration, making the immune system more robust as it makes use of the additional time by upregulating the production of B and T cells needed for greater immunological memory in the adaptive immune response. Third, adjuvants may help to increase the capacity to cause local reactions at the injection site (during vaccination), inducing greater release of danger signals by chemokine releasing cells such as helper T cells and mast cells. Fourth, they may induce the release of inflammatory cytokines which helps to not only recruit B and T cells at sites of infection but also to increase transcriptional events leading to a net increase of immune cells as a whole. Finally, adjuvants are believed to increase the innate immune response to antigen by interacting with pattern recognition receptors (PRRs) on or within accessory cells.

In embodiments, an adjuvant is used with the MSR. The adjuvant can be co-administered with the MSR or carried by the MSR. In aspects the adjuvant is selected from cytosine-guanosine oligonucleotide (CpG-ODN) sequence, granulocyte macrophage colony stimulating factor (GM-CSF), ovalbumin (OVA), monophosphoryl lipid A (MPL), poly(I: C), MF59, alum, aluminum hydroxide, aluminum phosphate, calcium phosphate hydroxide, Quil A, N-acetyl muramyl-L-alanyl-D-isoglutamine (MDP), FIA, montanide, adjuvant 65, lipovant, poly (DL-lactide-coglycolide) microspheres, paraffin oil, squalene, virosome, AS03, AS04, IL-1, IL-3, IL-4, IL-5, IL-6, IL-10, IL-12, IL-17, IL-18, Toll-like receptor ligand, CD40L, pathogen-associated molecular patterns (PAMPs), damage-associated molecular pattern molecules (DAMPs), Freund's complete adjuvant, Freund's incomplete adjuvant, antibodies against immune suppressive molecules, lipopolysaccharides (LPS), Fas ligand, Trail, lymphotactin, Mannan (M-FP), APG-2, Hsp70 and Hsp90.QS-21, Oxidized 1-palmitoyl-2-arachidonoyl-sn-glycero-3-phosphocholine, (Oxpapc) and alpha-Galactosylceramide. In aspects, a combination of adjuvants (i.e., more than one) is used in the methods described herein.

### Methods of Use

Accordingly, an aspect of the invention is to alter the therapeutic window and utilization of cytokine proteins to achieve improved outcomes in tumor treatment through the suppression, reduction, and in certain cases elimination of a tumor through the application of cytokine proteins to a targeted area.

It is a further aspect of the invention to alter the therapeutic window and utilization of cytokine proteins to achieve preferable outcomes in inflammation treatment through the suppression, reduction, and in certain cases elimination of an inflammatory region through the application of cytokine proteins to a targeted area. Conditions ameliorated by immune stimulation include, for example, infectious diseases, (e.g., bacterial, fungal, viral and parasitic infectious diseases). Also, conditions associated with uncontrolled proliferation of cells (e.g., cancers) can be ameliorated by immune stimulation.

Delivery of mesoporous silica rods may be accompanied by adjuvants which serve as secondary signals for inflammasome activation. Examples of adjuvants contemplated by the present invention include but are not limited to: Toll Like Receptor (“TLR”) agonists (e.g., TLR9), lipopolysaccharides (LPS), and aluminum hydroxide (alum).

The internal geometry of the mesoporous silica rod structure and mesoporous silica material can vary based on application. The internal structure may be labyrinthine (maze-like) or may contain internal pores of varying diameter to allow controlled influx of inflammatory response agents generated by the organism while metering egress of any of the therapeutic payloads and/or adjuvants described above. The internal geometry may also comprise several dead ends, as an internal pore does not necessarily have to lead to an aperture opening on the surface of the mesoporous silica rod or mesoporous silica structure, which therefore offers a time-release mechanism for excretion of therapeutic product.

The overall geometry of the mesoporous silica material is also not limited to that of a cylinder. Other common and equivalent shapes are contemplated, including but not limited to spheres, icosahedrons, bars, irregular rods, pyramids, cubes, cuboids, prisms, octahedrons, dodecahedrons, triangular prisms, octagonal prisms, pentagonal prisms, ellipsoids, irregular ellipsoids, tetrahedrons, square pyramids, hexagonal pyramids, and any other geometrical equivalents and substitutes known in the art.

### Methods of Manufacturing Mesoporous Silica Rods (MSRs)

In embodiments, the product includes mesoporous silica rods (MSRs) loaded (via adsorption) with the cytokine GM-CSF (Leukine®) and the adjuvant CpG 7909 (CpG). The dosage form can be a lyophilized powder that is reconstituted in water (WFI) prior to administration. As described above, the drug product can be administered via subcutaneous injection.

The MSRs can be 70-100 μm long with a width of 3-6 μm, and the unique structure of the material provides a high pore volume and large surface area, which allows component (Leukine and CpG) loading and controlled release. Synthetic amorphous silica is known to have a good biocompatibility toxicity profile and in situ dissolution/excretion properties.

Tertraethyl orthosilicate silica (TEOS) can be used as a raw material source of silicon dioxide in the manufacturing process. TEOS serves as a precursor to silicon dioxide. Accordingly, the TEOS material can be used in the manufacturing process to fabricate the mesoporous silica. As shown below, the TEOS converts to silicon dioxide upon the addition of water.

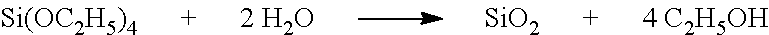

The rod structure of the mesoporous silica can be created using a poloxamer (e.g., pluronic P123 symmetric triblock copolymer) as a structural template on which the silicon dioxide can form a three-dimensional architecture. After the silica forms around the polymer template, the polymer can be removed using high temperature. The removal of the polymer leaves behind the longitudinal pores.

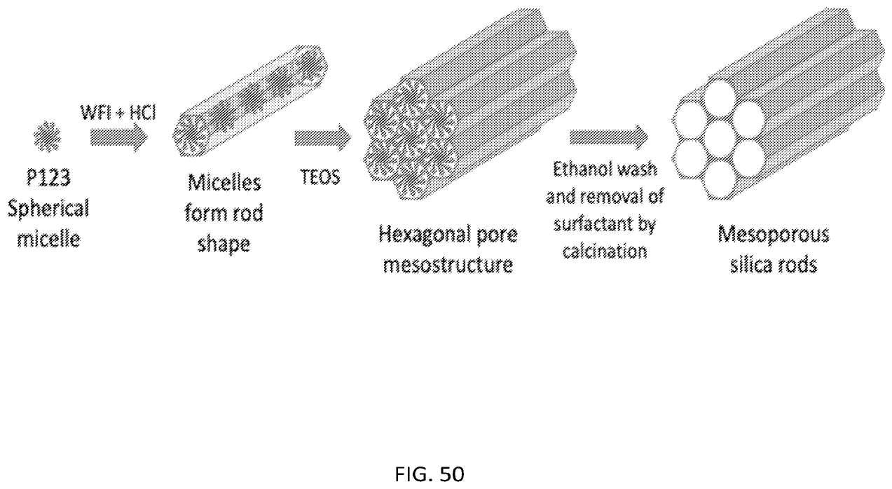

FIG. 50 shows an overview of a process of manufacturing mesoporous silica rod manufacture according to aspects of the invention. A symmetric triblock copolymer (Pluronic P123) composed of poly(ethylene oxide) and poly(propylene oxide) is employed to create form rod shaped micelles in solution. Tetraethyl Orthosilicate (TEOs) is added to the solution and the silica deposits on the micelles, creating a hexagonal pore structure. The Pluronic P123 is rinsed and calcinated (high temperature treatment) to remove the polymer, leaving a silica mesoporous structure.

The mesoporous silica rods are combined with the sterile filtered components (GM-CSF, CpG) and mixed well to allow adsorption. This solution is then placed in vials, frozen, lyophilized and stored at −20° C. The proposed configuration of the system will consist of a sterile vial which contains three components (MSR, GM-CSF & CpG) as a lyophillzed powder, which is reconstituted with water for injection.

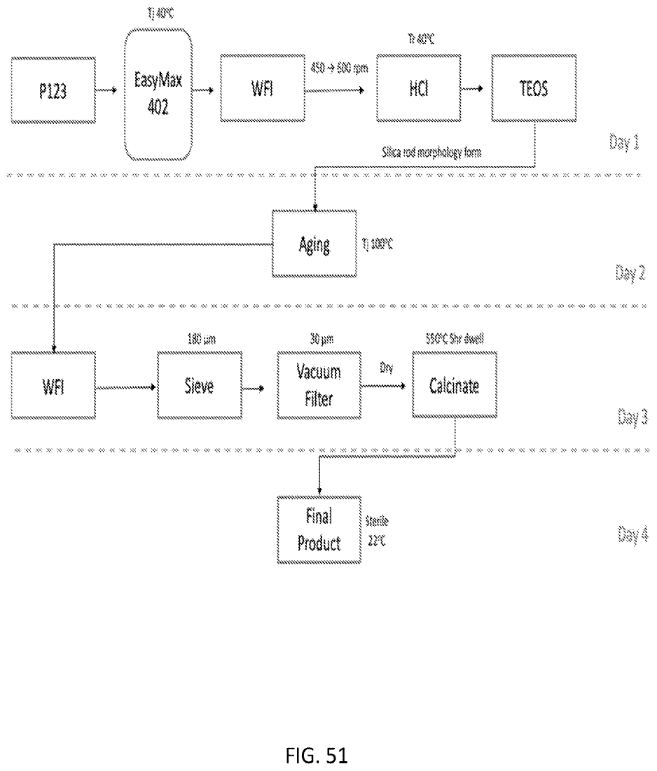

The processed solution is an aqueous admix, followed by lyophilization. FIG. 51 shows a detailed flowchart of a manufacturing process. Tⱼ refers to the jacket temperature and Tr refers to the internal temperature.

Pluronic P123 (P123) is heated to 40° C. using an EasyMax 402® system. Water for injection (WFI) is added and the solution is mixed (450 to 600 rpm). Acid (37% Hydrochloric acid) is added along with tetraethyl orthosilicate (TEOS). The silica rod morphology will form in the solution. The solution is left for about 48 hours (i.e., aged) at 100° C.

On day three, water (WFI) is added and the solution is sieved (180 μm) and then vacuum filtered (30 μm). The product is then dried (calcinated at 550° C. for 5 hours). The final produce can then be sterilized. As noted above, the product can be stored and shipped in lyophilized form. It remains stable at −20° C.

### Porosity/Volume Determination

The mesoporous silica rod material provides a large surface area to adsorb the components of the system that can facilitate controlled delivery once injected into the subcutaneous space.

The pore size distribution analysis is conducted using a static pressure (volumetric) analyzer using the gas adsorption technique. The amount of inert gas adsorbed to the surface of a sample is measured at varying relative pressures by this technique. Once the pressure is reduced incrementally (desorption), the condensed gas evaporates from the pores. From the resulting isotherm, the Barrett, Joyner, and Halenda (BJH) theory is used to determine the cylindrical equivalent pore volumes and pore areas from the amount of gas adsorbed and desorbed. The surface area can also be determined using the Brunauer-Emmett-Teller (BET) theory.

### Silica Purity (Thermo-Gravimetric Analysis)

The thermal mass loss profile of the samples is measured via thermo-gravimetric analysis (TGA). The instrument utilizes a microbalance encased within a furnace that utilizes a sample carrier and thermocouple combination to accurately record changes in sample mass over time as the temperature is increased. The mass change over time can also be observed as the sample can be held constant at a temperature of interest. As the mesoporous silica consists on a high percent of SiO2, the TGA will provide the percent of the sample that is silica.

### Impurities (Inductively Coupled Plasma Mass Spectroscopy)

Inductively coupled plasma mass spectroscopy (ICP-MS) is an analytical technique for determining trace multi-elemental and isotopic concentrations in liquid, solid, or gaseous samples. It combines an ion-generating argon plasma source with the sensitive detection limit of mass spectrometry detection. This technique will be utilized to identify the absence of residual metals and carbon-based impurities in the mesoporous silica.

### Silica Dimensions (Malvern® Morphologi G3S Image Analyzer)

The particle size and shape analysis of the MSRs will be conducted on a Malvern® Morphologi G3S or Malvern® Morphologi 4 image analyzer. This instrument is an automated microscope that uses a series of objectives of varying magnifications, a motorized stage, and a digital camera to capture images of particles to determine particle size and shape. The analytical range for this technique is approximately 0.50 μm to 1,000 μm, though measurements up to 10,000 μm can be achieved for certain applications.

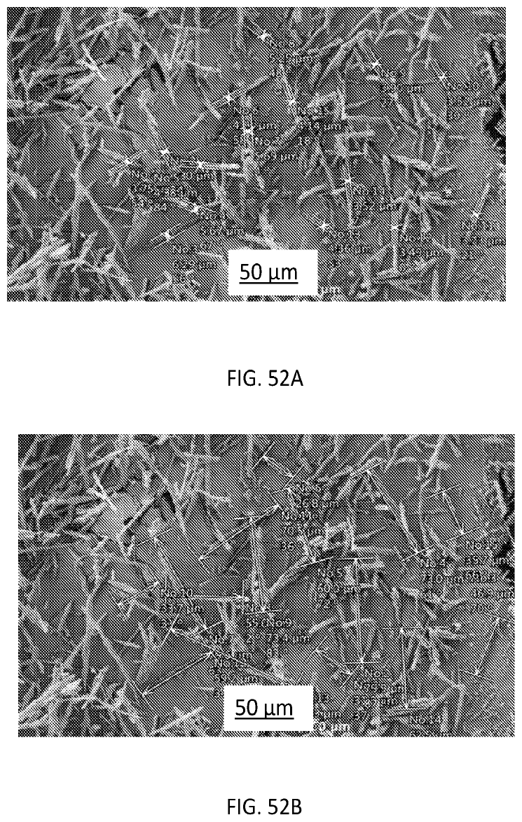

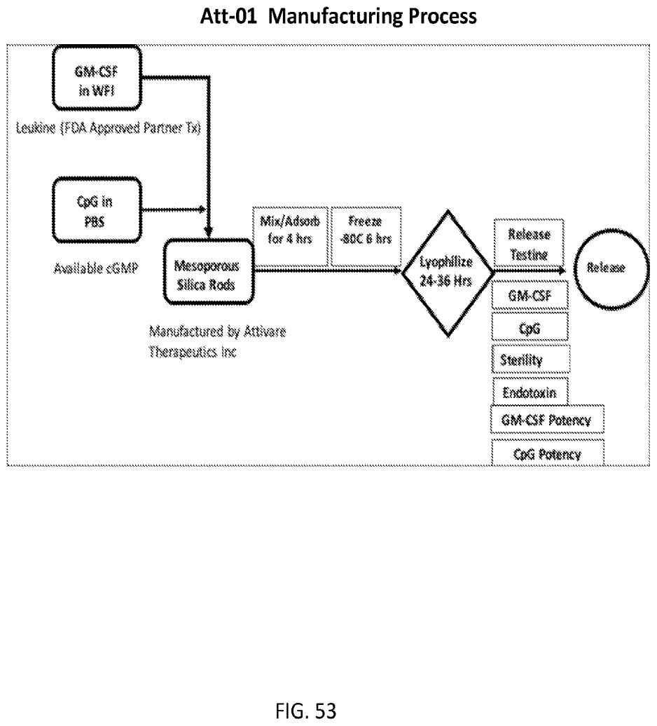

The instrument captures an image (see FIGS. 52A and 52B) of the particle as it passes into the chosen objective's field of view. The instrument determines the size of an individual pixel for the chosen magnification and creates a projected two-dimensional image. The instrument then converts the pixels of the two-dimensional image into a circle that has the same pixel area as the two-dimensional image, thus reporting the circular equivalent (CE) diameter.

FIGS. 52A and 52B are scanning electron microscope (SEM) sizing images of Standard MSR length and width as analyzed by Malvern® Morphologi G3S instrument. Standard MSRs length (left) and width (right). D[n,0.1] (μm), D[n,0.5] (μm) D[n,0.9] (μm)=biodistribution of MSRs sized at 10% or less, 50% or less, and 90% or less respectively within the total number mean.

Important functional properties of the mesoporous silica rods are related to the physical properties, the dimensions, surface area, pore size and volume, which are included in the release criteria. In addition, the identity and purity of the material (SIO2) is achieved by Inductively Coupled Plasma Mass Spectrometry (ICP-MS), which will identify the elemental content of the base material. Increasing the temperature and comparing the combustion thermal transitions to pure components can reveal its chemical composition. TGA is used to determine the purity of synthesized nanomaterials by comparing them to standards.

In aspects, the following routine tests are applied to the mesoporous silica rods:

TABLE 1
Proposed Specification for Mesoporous Silica Rods

| | | Proposed | |
|---|---|---|---|
| Characteristic | Parameter or Test Method | Specification | |
| Appearance (Color) | Visual | White | |
| Appearance (Form) | Visual | Powder | |
| Average length | Malvern Morphologi | 80-120 | μm |
| | G3S/4 Image Analysis | | |
| Average width | Malvern Morphologi | <10 | μm |
| | G3S/4 Image Analysis | | |
| Material Surface | BET Surface Area Analysis (BET) <ISO | 500-1000 | m²/g |
| area | 9277:2010> | | |
| Average pore | Porosimetry Analysis by Gas Adsorption | 1-10 | nm |
| diameter | (PoreGA) ISO-9277 | | |
| Pore volume | Porosimetry Analysis by Gas Adsorption | 0.1-2 | cm³/g |

| | | |
|---|---|---|
| | (PoreGA) ISO-9277 | |
| Purity | Thermogravimetric analysis (TGA) | >95% |
| Impurities | Inductively Coupled Plasma Mass | TBD |
| (Elemental) | Spectrometry (ICP-MS) | |
| Endotoxin (EU/ml) | USP<85>/EP 2.6.14 | ≤5 |
| Sterility | USP<71> | No Growth |
| Water Content | Karl Fischer Titrator | <1% |

### Administration

The mesoporous silica structure may also be administered alone to create a physical insult to the tumor or inflammatory area, and thus spur an innate immune response from the human body or organism, which in effect will lead to tumor regression through natural cellular processes such as phagocytosis.

Various sized molecules of therapeutics are also contemplated by the present invention and can be employed within the mesoporous silica rod structure or mesoporous silica material. Various sized molecules may also be applied to the outer surface of the mesoporous silica rod structure or mesoporous silica material as a coating. Furthermore, various molecules may be combined with any number of other therapeutic agents, adjuvants, prodrugs, buffers, agents, and the like.

The cytokine payloads of the invention may be directly injected into a targeted tumor area and/or injected adjacent to the targeted tumor area.

The mesoporous silica rod structure or mesoporous silica material can have at least 6 pores designated for excretion of therapeutic payloads. Alternatively, the mesoporous silica rod structure or silica material can have at least 1 pore, no more than 2 pores, no more than 3 pores, no more than 4 pores, no more than 5 pores, no more than 6 pores, no more than 7 pores, no more than 8 pores, no more than 9 pores, no more than 10 pores, no more than 11 pores, no more than 12 pores, no more than 13 pores, no more than 14 pores, no more than 15 pores, no more than 20 pores, no more than 25 pores, no more than 30 pores, no more than 40 pores, no more than 50 pores, no more than 60 pores, no more than 75 pores, no more than 100 pores, no more than 120 pores, no more than 150 pores, no more than 175 pores, no more than 200 pores, no more than 250 pores, no more than 300 pores, no more than 350 pores, no more than 400 pores, no more than 500 pores, no more than 600 pores, no more than 750 pores, no more than 1,000 pores, no more than 1,200 pores, no more than 1,500 pores, no more than 2,000 pores, no more than 3,000 pores, no more than 5,000 pores, no more than 10,000 pores, no more than 15,000 pores, no more than 20,000 pores, no more than 30,000 pores, no more than 50,000 pores, no more than 100,000 pores, no more than 1,000,000 pores.

The mesoporous silica rod structure or mesoporous silica material can have at least 7 pores designated for excretion of therapeutic payloads. Alternatively, the mesoporous silica rod structure or silica material can have at least 8 pores, at least 9 pores, at least 10 pores, at least 11 pores, at least 12 pores, at least 13 pores, at least 14 pores, at least 15 pores, at least 20 pores, at least 25 pores, at least 30 pores, at least 40 pores, at least 50 pores, at least 60 pores, at least 75 pores, at least 100 pores, at least 120 pores, at least 150 pores, at least 175 pores, at least 200 pores, at least 250 pores, at least 300 pores, at least 350 pores, at least 400 pores, at least 500 pores, at least 600 pores, at least 750 pores, at least 1,000 pores, at least 1,200 pores, at least 1,500 pores, at least 2,000 pores, at least 3,000 pores, at least 5,000 pores, at least 10,000 pores, at least 15,000 pores, at least 20,000 pores, at least 30,000 pores, at least 50,000 pores, at least 100,000 pores, at least 1,000,000 pores.

The pores of the mesoporous silica rod structure or mesoporous silica material can have uniform diameters, or in other aspects possess varying diameters. In yet other aspects, the pores of the mesoporous silica rod structure may possess an array of diameters arranged in a pattern of alternating diameters and dimensions. In yet other aspects, the varying pore diameter of the mesoporous silica rod structure may be created and determined randomly either by human input, an algorithm, a recursive algorithm, a computer program, by nano-machining, by nano-engineering, by 3-D printing, by 3-D printing programs, through chemical processes, and all other processes and methods of design suitable for the purposes of the present invention and well known and recognized by the art.

Therapeutic payloads include all of the payloads described above, as well as any other art-recognized equivalent or any therapeutic known in the art.

The mesoporous silica rod structure or mesoporous silica material can also dissolve within the target area and/or remain localized to the site of treatment.

The mesoporous silica rod structure or mesoporous silica material can carry or be coated with therapeutic payloads, and these therapeutic payloads may be a combination of all of the substances, drugs, adjuvants, and proteins described herein as well as all art-known equivalents and therapeutic equivalents recognized in the art. In some embodiments, the MSRs have one or more surface modifications (e.g., treated with a substance such as glycolic acid or lactic acid, have been conjugated to an amine, thiol, chloro, or phosphonate group, or a compound such as PEI has been added to the MPS rods). In embodiments a surface modified MSR is an MSR in which free PEI has been added.

In certain aspects, the pore size of the mesoporous silica material and mesoporous silica rod disclosed herein is about 2 nm (nanometers), about 3 nm to 50 nm, from 36 nm to 50 nm, from 37 nm to 50 nm, from 38 nm to 50 nm, from 39 nm to 50 nm, from 40 nm to 50 nm, from 41 nm to 50 nm, from 42 nm to 50 nm, from 43 nm to 50 nm, from 44 nm to 50 nm, from 45 nm to 50 nm, from 46 nm to 50 nm, from 47 nm to 50 nm, from 48 nm to 50 nm, from 49 nm to 50 nm.

In certain aspects of the present invention, the pore size of the mesoporous silica material and mesoporous silica rod disclosed herein can range from 2 nm to 3 nm, from 2 nm to 4 nm, from 2 nm to 5 nm, from 2 nm to 10 nm, from 2 nm to 15 nm, from 2 nm to 20 nm, from 2 nm to 25 nm, from 2 nm to 30 nm, from 2 nm to 35 nm, from 2 nm to 40 nm, from 2 nm to 45 nm, from 5 nm to 10 nm, from 5 nm to 15 nm, from 5 nm to 20 nm, from 5 nm to 25 nm, from 5 nm to 30 nm, from 5 nm to 35 nm, from 5 nm to 40 nm, from 5 nm to 45 nm, from 5 nm to 50 nm, from 10 nm to 15 nm, from 10 nm to 20 nm, from 10 nm to 25 nm, from 10 nm to 30 nm, from 10 nm to 35 nm, from 10 nm to 40 nm, from 10 nm to 45 nm, from 10 nm to 50 nm, from 15 nm to 20 nm, from 15 nm to 25 nm, from 15 nm to 30 nm, from 15 nm to 35 nm, from 15 nm to 40 nm, from 15 nm to 45 nm, from 20 nm to 25 nm, from 20 nm to 30 nm, from 20 nm to 35 nm, from 20 nm to 40 nm, from 20 nm to 45 nm, from 25 nm to 30 nm, from 25 nm to 35 nm, from 25 nm to 40 nm, from 25 nm to 45 nm, from 30 nm to 35 nm, from 30 nm to 40 nm, from 30 nm to 45 nm, from 35 nm to 40 nm, from 35 nm to 45 nm, from 40 nm to 45 nm.

In certain aspects of the present invention, the pore size of the mesoporous silica material and mesoporous silica rod disclosed herein can be about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 20 nm, about 25 nm, about 30 nm, about 35 nm, about 40 nm, about 45 nm, about 50 nm, about 55 nm, about 60 nm, about 70 nm, about 80 nm, about 90 nm, about 100 nm, about 110 nm, about 120 nm, about 120 nm, about 130 nm, about 140 nm, about 150 nm, about 160 nm, about 175 nm, about 190 nm, about 200 nm, about 215 nm, about 225 nm, about 250 nm, about 275 nm, about 300 nm or more.

In certain aspects of the present invention, the pore size of the mesoporous silica material and mesoporous silica rod disclosed herein can be no more than 2 nm, no more than 3 nm, no more than 4 nm, no more than 5 nm, no more than 6 nm, no more than 7 nm, no more than 8 nm, no more than 9 nm, no more than 10 nm, no more than 11 nm, no more than 12 nm, no more than 13 nm, no more than 14 nm, no more than 15 nm, no more than 20 nm, no more than 25 nm, no more than 30 nm, no more than 35 nm, no more than 40 nm, no more than 45 nm, no more than 50 nm, no more than 55 nm, no more than 60 nm, no more than 70 nm, no more than 80 nm, no more than 90 nm, no more than 100 nm, no more than 110 nm, no more than 120 nm, no more than 120 nm, no more than 130 nm, no more than 140 nm, no more than 150 nm, no more than 160 nm, no more than 175 nm, no more than 190 nm, no more than 200 nm, no more than 215 nm, no more than 225 nm, no more than 250 nm, no more than 275 nm, no more than 300 nm, no more than 325 nm, no more than 350 nm, no more than 500 nm.

In certain aspects of the present invention, the pore size of the mesoporous silica material and mesoporous silica rod disclosed herein can be at least 2 nm, at least 3 nm, at least 4 nm, at least 5 nm, at least 6 nm, at least 7 nm, at least 8 nm, at least 9 nm, at least 10 nm, at least 11 nm, at least 12 nm, at least 13 nm, at least 14 nm, at least 15 nm, at least 20 nm, at least 25 nm, at least 30 nm, at least 35 nm, at least 40 nm, at least 45 nm, at least 50 nm, at least 55 nm, at least 60 nm, at least 70 nm, at least 80 nm, at least 90 nm, at least 100 nm, at least 110 nm, at least 120 nm, at least 120 nm, at least 130 nm, at least 140 nm, at least 150 nm, at least 160 nm, at least 175 nm, at least 190 nm, at least 200 nm, at least 215 nm, at least 225 nm, at least 250 nm, at least 275 nm, at least 300 nm, at least 325 nm, at least 350 nm, at least 500 nm.

In certain aspects of the present invention, the pore size of the mesoporous silica material and mesoporous silica rod disclosed herein can range from 2 nm to 55 nm, from 2 nm to 60 nm, from 2 nm to 65 nm, from 2 nm to 70 nm, from 2 nm to 75 nm, from 2 nm to 80 nm, from 2 nm to 85 nm, from 2 nm to 90 nm, from 2 nm to 95 nm, from 2 nm to 100 nm, from 2 nm to 105 nm, from 2 nm to 110 nm, from 2 nm to 115 nm, from 2 nm to 120 nm, from 5 nm to 55 nm, from 5 nm to 60 nm, from 5 nm to 65 nm, from 5 nm to 70 nm, from 5 nm to 75 nm, from 5 nm to 80 nm, from 5 nm to 85 nm, from 5 nm to 90 nm, from 5 nm to 95 nm, from 5 nm to 100 nm, from 5 nm to 105 nm, from 5 nm to 110 nm, from 5 nm to 115 nm, from 5 nm to 120 nm, from 10 nm to 55 nm, from 10 nm to 60 nm, from 10 nm to 65 nm, from 10 nm to 70 nm, from 10 nm to 75 nm, from 10 nm to 80 nm, from 10 nm to 85 nm, from 10 nm to 90 nm, from 10 nm to 95 nm, from 10 nm to 100 nm, from 10 nm to 105 nm, from 10 nm to 110 nm, from 10 nm to 115 nm, from 10 nm to 120 nm, from 20 nm to 55 nm, from 20 nm to 60 nm, from 20 nm to 65 nm, from 20 nm to 70 nm, from 20 nm to 75 nm, from 20 nm to 80 nm, from 20 nm to 85 nm, from 20 nm to 90 nm, from 20 nm to 95 nm, from 20 nm to 100 nm, from 20 nm to 105 nm, from 20 nm to 110 nm, from 20 nm to 115 nm, from 20 nm to 120 nm, from 30 nm to 55 nm, from 30 nm to 60 nm, from 30 nm to 65 nm, from 30 nm to 70 nm, from 30 nm to 75 nm, from 30 nm to 80 nm, from 30 nm to 85 nm, from 30 nm to 90 nm, from 30 nm to 95 nm, from 30 nm to 100 nm, from 30 nm to 105 nm, from 30 nm to 110 nm, from 30 nm to 115 nm, from 30 nm to 120 nm, from 40 nm to 55 nm, from 40 nm to 60 nm, from 40 nm to 65 nm, from 40 nm to 70 nm, from 40 nm to 75 nm, from 40 nm to 80 nm, from 40 nm to 85 nm, from 40 nm to 90 nm, from 40 nm to 95 nm, from 40 nm to 100 nm, from 40 nm to 105 nm, from 40 nm to 110 nm, from 40 nm to 115 nm, from 40 nm to 120 nm, from 50 nm to 55 nm, from 50 nm to 60 nm, from 50 nm to 65 nm, from 50 nm to 70 nm, from 50 nm to 75 nm, from 50 nm to 80 nm, from 50 nm to 85 nm, from 50 nm to 90 nm, from 50 nm to 95 nm, from 50 nm to 100 nm, from 50 nm to 105 nm, from 50 nm to 110 nm, from 50 nm to 115 nm, from 50 nm to 120 nm, from 60 nm to 65 nm, from 60 nm to 70 nm, from 60 nm to 75 nm, from 60 nm to 80 nm, from 60 nm to 85 nm, from 60 nm to 90 nm, from 60 nm to 95 nm, from 60 nm to 100 nm, from 60 nm to 105 nm, from 60 nm to 110 nm, from 60 nm to 115 nm, from 60 nm to 120 nm, from 70 nm to 75 nm, from 70 nm to 80 nm, from 70 nm to 85 nm, from 70 nm to 90 nm, from 70 nm to 95 nm, from 70 nm to 100 nm, from 70 nm to 105 nm, from 70 nm to 110 nm, from 70 nm to 115 nm, from 70 nm to 120 nm, from 80 nm to 85 nm, from 80 nm to 90 nm, from 80 nm to 95 nm, from 80 nm to 100 nm, from 80 nm to 105 nm, from 80 nm to 110 nm, from 80 nm to 115 nm, from 80 nm to 120 nm, from 90 nm to 95 nm, from 90 nm to 100 nm, from 90 nm to 105 nm, from 90 nm to 110 nm, from 90 nm to 115 nm, from 90 nm to 120 nm, from 100 nm to 105 nm, from 100 nm to 110 nm, from 100 nm to 115 nm, from 100 nm to 120 nm, from 110 nm to 115 nm, from 110 nm to 120 nm.

In certain aspects of the present invention, the pore size of the mesoporous silica material and mesoporous silica rod disclosed herein can range from about 2 nm to about 50 nm, from about 2 nm to about 5 nm, from about 2 nm to about 10 nm, from about 2 nm to about 15 nm, from about 2 nm to about 20 nm, from about 2 nm to about 30 nm, from about 2 nm to about 40 nm, from about 5 nm to about 10 nm, from about 5 nm to about 15 nm, from about 5 nm to about 20 nm, from about 5 nm to about 30 nm, from about 5 nm to about 40 nm, from about 5 nm to about 50 nm, from about 10 nm to about 15 nm, from about 10 nm to about 20 nm, from about 10 nm to about 25 nm, from about 10 nm to about 30 nm, from about 10 nm to about 40 nm, from about 10 nm to about 50 nm, from about 15 nm to about 20 nm, from about 15 nm to about 25 nm, from about 15 nm to about 30 nm, from about 15 nm to about 40 nm, from about 15 nm to about 50 nm, from about 20 nm to about 25 nm, from about 20 nm to about 30 nm, from about 20 nm to about 35 nm, from about 20 nm to about 40 nm, from about 20 nm to about 50 nm, from about 25 nm to about 30 nm, from about 25 nm to about 35 nm, from about 25 nm to about 40 nm, from about 25 nm to about 50 nm, from about 30 nm to about 35 nm, from about 30 nm to about 40 nm, from about 30 nm to about 45 nm, from about 30 nm to about 50 nm, from about 35 nm to about 40 nm, from about 35 nm to about 45 nm, from about 35 nm to about 50 nm, from about 40 nm to about 45 nm, from about 40 nm to about 50 nm, from about 45 nm to about 50 nm, from about 46 nm to about 50 nm, from about 47 nm to about 50 nm, from about 48 nm to about 50 nm, from about 49 nm to about 50 nm.

In certain aspects of the present invention, the pore size of the mesoporous silica material and mesoporous silica rod disclosed herein can range from about 2 nm to about 50 nm, from about 2 nm to about 55 nm, from about 2 nm to about 60 nm, from about 2 nm to about 65 nm, from about 2 nm to about 65 nm, from about 2 nm to about 80 nm, from about 2 nm to about 100 nm, from about 2 nm to about 120 nm, from about 2 nm to about 150 nm, from about 2 nm to about 200 nm, from about 5 nm to about 55 nm, from about 5 nm to about 60 nm, from about 5 nm to about 65 nm, from about 5 nm to about 80 nm, from about 5 nm to about 100 nm, from about 5 nm to about 120 nm, from about 5 nm to about 150 nm, from about 10 nm to about 55 nm, from about 10 nm to about 60 nm, from about 10 nm to about 65 nm, from about 10 nm to about 80 nm, from about 10 nm to about 100 nm, from about 10 nm to about 120 nm, from about 10 nm to about 150 nm, from about 15 nm to about 55 nm, from about 15 nm to about 60 nm, from about 15 nm to about 80 nm, from about 15 nm to about 100 nm, from about 15 nm to about 120 nm, from about 15 nm to about 150 nm, from about 20 nm to about 55 nm, from about 20 nm to about 60 nm, from about 20 nm to about 65 nm, from about 20 nm to about 80 nm, from about 20 nm to about 100 nm, from about 20 nm to about 100 nm, from about 20 nm to about 120 nm, from about 20 nm to about 150 nm, from about 30 nm to about 55 nm, from about 30 nm to about 80 nm, from about 30 nm to about 100 nm, from about 30 nm to about 120 nm, from about 30 nm to about 150 nm, from about 40 nm to about 55 nm, from about 40 nm to about 80 nm, from about 40 nm to about 100 nm, from about 40 nm to about 120 nm, from about 40 nm to about 150 nm, from about 50 nm to about 60 nm, from about 50 nm to about 65 nm, from about 50 nm to about 80 nm, from about 50 nm to about 100 nm, from about 50 nm to about 120 nm, from about 50 nm to about 150 nm, from about 60 nm to about 65 nm, from about 60 nm to about 70 nm, from about 60 nm to about 80 nm, from about 60 nm to about 100 nm, from about 60 nm to about 120 nm, from about 60 nm to about 150 nm, from about 70 nm to about 75 nm, from about 70 nm to about 80 nm, from about 70 nm to about 100 nm, from about 70 nm to about 110 nm, from about 70 nm to about 120 nm, from about 70 nm to about 130 nm, from about 70 nm to about 140 nm, from about 70 nm to about 150 nm, from about 80 nm to about 85 nm, from about 80 nm to about 90 nm, from about 80 nm to about 100 nm, from about 80 nm to about 120 nm, from about 80 nm to about 150 nm, from about 90 nm to about 100 nm, from about 90 nm to about 110 nm, from about 90 nm to about 120 nm, from about 90 nm to about 150 nm, from about 100 nm to about 110 nm, from about 100 nm to about 120 nm, from about 100 nm to about 130 nm, from about 100 nm to about 140 nm, from about 100 nm to about 150 nm, from about 110 nm to about 120 nm, from about 110 nm to about 140 nm, from about 110 nm to about 150 nm, from about 120 nm to about 135 nm, from about 120 nm to about 150 nm, from about 130 nm to about 140 nm, from about 130 nm to about 150 nm, from about 140 nm to about 145 nm, from about 140 nm to about 150 nm.

The dosage of payload, silica material, cytokine protein, inflammasome, interleukin, adjuvant, pharmaceutical composition, therapeutic, prodrug, and other equivalents of the present invention can range from 1 ng (nanogram) to about 100 ng, from 1 ng to about 500 ng, from 1 ng to about 1 μg (microgram), from 1 ng to about 2 μg, from about 1 ng to about 3 μg, from about 1 ng to about 4 μg, from about 1 ng to about 5 μg, from about 1 ng to about 6 μg, from about 1 ng to about 7 μg, from about 1 ng to about 8 μg, from about 1 ng to about 9 μg, from about 1 ng to about 10 μg, from about 1 ng to about 12 μg, from about 1 ng to about 15 μg, from about 1 ng to about 17 μg, from about 1 ng to about 20 μg, from about 1 ng to about 25 μg, from about 1 ng to about 30 μg, from about 1 ng to about 35 μg, from about 1 ng to about 40 μg, from about 1 ng to about 50 μg, from about 1 ng to about 60 μg, from about 1 ng to about 75 μg, from about 1 ng to about 100 μg, from about 1 ng to about 1 mg, from about 1 ng to about 1 g.

In other aspects, the dosage of payload, silica material, cytokine protein, inflammasome, interleukin, adjuvant, pharmaceutical composition, therapeutic, prodrug, and other equivalents of the present invention can range from about 1 μg to about 2 μg, from about 1 μg to about 3 μg, from about 1 μg to about 4 μg, from about 1 μg to about 5 μg, from about 1 μg to about 6 μg, from about 1 μg to about 8 μg, from about 1 μg to about 10 μg, from about 1 μg to about 15 μg, from about 1 μg to about 20 μg, from about 1 μg to about 30 μg, from about 1 μg to about 40 μg, from about 1 μg to about 50 μg, from about 1 μg to about 100 μg, from about 1 μg to about 500 μg, from about 1 μg to about 1 mg, from about 1 μg to about 5 mg, from about 1 μg to about 10 mg, from about 1 μg to about 20 mg, from about 1 μg to about 50 mg, from about 1 μg to about 100 mg, from about 1 μg to about 200 mg, from about 1 μg to about 500 mg, from about 1 μg to about 750 mg, from about 1 μg to about 1 g.

The dosage of payload, silica material, cytokine protein, inflammasome, interleukin, adjuvant, pharmaceutical composition, therapeutic, prodrug, and other equivalents of the present invention can be about 1 ng, about 2 ng, about 3 ng, about 4 ng, about 5 ng, about 6 ng, about 7 ng, about 8 ng, about 9 ng, about 10 ng, about 11 ng, about 12 ng, about 13 ng, about 14 ng, about 15 ng, about 16 ng, about 17 ng, about 19 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 110 ng, about 120 ng, about 130 ng, about 140 ng, about 150 ng, about 160 ng, about 175 ng, about 190 ng, about 200 ng, about 225 ng, about 250 ng, about 275 ng, about 300 ng, about 325 ng, about 350 ng, about 375 ng, about 400 ng, about 425 ng, about 450 ng, about 475 ng, about 500 ng, about 550 ng, about 600 ng, about 650 ng, about 700 ng, about 750 ng, about 800 ng, about 850 ng, about 900 ng, about 950 ng, about 1,000 ng, about 1,200 ng, about 1,500 ng, about 2,000 ng, about 3,500 ng, about 5,000 ng.

The dosage of payload, silica material, cytokine protein, inflammasome, interleukin, adjuvant, pharmaceutical composition, therapeutic, prodrug, and other equivalents of the present invention can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 30 μg, about 32 μg, about 35 μg, about 37 μg, about 40 μg, about 42 μg, about 45 μg, about 48 μg, about 50 μg, about 55 μg, about 60 μg, about 65 μg, about 70 μg, about 75 μg, about 80 μg, about 85 μg, about 90 μg, about 95 μg, about 100 μg, about 110 μg, about 120 μg, about 130 μg, about 140 μg, about 150 μg, about 160 μg, about 170 μg, about 180 μg, about 190 μg, about 200 μg, about 215 μg, about 230 μg, about 250 μg, about 265 μg, about 275 μg, about 300 μg, about 325 μg, about 350 μg, about 375 μg, about 400 μg, about 425 μg, about 450 μg, about 475 μg, about 500 μg, about 550 μg, about 600 μg, about 650 μg, about 700 μg, about 750 μg, about 800 μg, about 850 μg, about 900 μg, about 950 μg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 22 mg, about 24 mg, about 25 mg, about 27 mg, about 29 mg, about 30 mg, about 33 mg, about 35 mg, about 37 mg, about 40 mg, about 43 mg, about 45 mg, about 48 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 220 mg, about 240 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 11 g, about 12 g, about 13 g, about 14 g, about 15 g, about 16 g, about 17 g, about 18 g, about 19 g, about 20 g, about 22 g, about 24 g, about 25 g, about 27 g, about 28 g, about 30 g, about 33 g, about 35 g, about 38 g, about 40 g, about 43 g, about 45 g, about 48 g, about 50 g, about 55 g, about 60 g, about 65 g, about 70 g, about 75 g, about 80 g, about 85 g, about 90 g, about 95 g, about 100 g.

The dosage of payload, silica material, cytokine protein, inflammasome, interleukin, adjuvant, pharmaceutical composition, therapeutic, prodrug, and other equivalents of the present invention can be at least 1 ng, at least 2 ng, at least 3 ng, at least 4 ng, at least 5 ng, at least 6 ng, at least 7 ng, at least 8 ng, at least 9 ng, at least 10 ng, at least 11 ng, at least 12 ng, at least 13 ng, at least 14 ng, at least 15 ng, at least 16 ng, at least 17 ng, at least 19 ng, at least 20 ng, at least 25 ng, at least 30 ng, at least 35 ng, at least 40 ng, at least 45 ng, at least 50 ng, at least 55 ng, at least 60 ng, at least 65 ng, at least 70 ng, at least 75 ng, at least 80 ng, at least 85 ng, at least 90 ng, at least 95 ng, at least 100 ng, at least 110 ng, at least 120 ng, at least 130 ng, at least 140 ng, at least 150 ng, at least 160 ng, at least 175 ng, at least 190 ng, at least 200 ng, at least 225 ng, at least 250 ng, at least 275 ng, at least 300 ng, at least 325 ng, at least 350 ng, at least 375 ng, at least 400 ng, at least 425 ng, at least 450 ng, at least 475 ng, at least 500 ng, at least 550 ng, at least 600 ng, at least 650 ng, at least 700 ng, at least 750 ng, at least 800 ng, at least 850 ng, at least 900 ng, at least 950 ng, at least 1,000 ng, at least 1,200 ng, at least 1,500 ng, at least 2,000 ng, at least 3,500 ng, at least 5,000 ng.

The dosage of payload, silica material, cytokine protein, inflammasome, interleukin, adjuvant, pharmaceutical composition, therapeutic, prodrug, and other equivalents of the present invention can be at least 1 μg, at least 2 μg, at least 3 μg, at least 4 μg, at least 5 μg, at least 6 μg, at least 7 μg, at least 8 μg, at least 9 μg, at least 10 μg, at least 11 μg, at least 12 μg, at least 13 μg, at least 14 μg, at least 15 μg, at least 16 μg, at least 17 μg, at least 18 μg, at least 19 μg, at least 20 μg, at least 21 μg, at least 22 μg, at least 23 μg, at least 24 μg, at least 25 μg, at least 30 μg, at least 32 μg, at least 35 μg, at least 37 μg, at least 40 μg, at least 42 μg, at least 45 μg, at least 48 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 110 μg, at least 120 μg, at least 130 μg, at least 140 μg, at least 150 μg, at least 160 μg, at least 170 μg, at least 180 μg, at least 190 μg, at least 200 μg, at least 215 μg, at least 230 μg, at least 250 μg, at least 265 μg, at least 275 μg, at least 300 μg, at least 325 μg, at least 350 μg, at least 375 μg, at least 400 μg, at least 425 μg, at least 450 μg, at least 475 μg, at least 500 μg, at least 550 μg, at least 600 μg, at least 650 μg, at least 700 μg, at least 750 μg, at least 800 μg, at least 850 μg, at least 900 μg, at least 950 μg, at least 1 mg, at least 1.5 mg, at least 2 mg, at least 2.5 mg, at least 3 mg, at least 3.5 mg, at least 4 mg, at least 4.5 mg, at least 5 mg, at least 5.5 mg, at least 6 mg, at least 6.5 mg, at least 7 mg, at least 7.5 mg, at least 8 mg, at least 8.5 mg, at least 9 mg, at least 9.5 mg, at least 10 mg, at least 11 mg, at least 12 mg, at least 13 mg, at least 14 mg, at least 15 mg, at least 16 mg, at least 17 mg, at least 18 mg, at least 19 mg, at least 20 mg, at least 22 mg, at least 24 mg, at least 25 mg, at least 27 mg, at least 29 mg, at least 30 mg, at least 33 mg, at least 35 mg, at least 37 mg, at least 40 mg, at least 43 mg, at least 45 mg, at least 48 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 110 mg, at least 120 mg, at least 130 mg, at least 140 mg, at least 150 mg, at least 160 mg, at least 170 mg, at least 180 mg, at least 190 mg, at least 200 mg, at least 220 mg, at least 240 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 550 mg, at least 600 mg, at least 650 mg, at least 700 mg, at least 750 mg, at least 800 mg, at least 850 mg, at least 900 mg, at least 950 mg, at least 1 g, at least 1.5 g, at least 2 g, at least 2.5 g, at least 3 g, at least 3.5 g, at least 4 g, at least 4.5 g, at least 5 g, at least 5.5 g, at least 6 g, at least 6.5 g, at least 7 g, at least 7.5 g, at least 8 g, at least 8.5 g, at least 9 g, at least 9.5 g, at least 10 g, at least 11 g, at least 12 g, at least 13 g, at least 14 g, at least 15 g, at least 16 g, at least 17 g, at least 18 g, at least 19 g, at least 20 g, at least 22 g, at least 24 g, at least 25 g, at least 27 g, at least 28 g, at least 30 g, at least 33 g, at least 35 g, at least 38 g, at least 40 g, at least 43 g, at least 45 g, at least 48 g, at least 50 g, at least 55 g, at least 60 g, at least 65 g, at least 70 g, at least 75 g, at least 80 g, at least 85 g, at least 90 g, at least 95 g, at least 100 g.

The dosage of payload, silica material, cytokine protein, inflammasome, interleukin, adjuvant, pharmaceutical composition, therapeutic, prodrug, and other equivalents of the present invention can be no more than 1 ng, no more than 2 ng, no more than 3 ng, no more than 4 ng, no more than 5 ng, no more than 6 ng, no more than 7 ng, no more than 8 ng, no more than 9 ng, no more than 10 ng, no more than 11 ng, no more than 12 ng, no more than 13 ng, no more than 14 ng, no more than 15 ng, no more than 16 ng, no more than 17 ng, no more than 19 ng, no more than 20 ng, no more than 25 ng, no more than 30 ng, no more than 35 ng, no more than 40 ng, no more than 45 ng, no more than 50 ng, no more than 55 ng, no more than 60 ng, no more than 65 ng, no more than 70 ng, no more than 75 ng, no more than 80 ng, no more than 85 ng, no more than 90 ng, no more than 95 ng, no more than 100 ng, no more than 110 ng, no more than 120 ng, no more than 130 ng, no more than 140 ng, no more than 150 ng, no more than 160 ng, no more than 175 ng, no more than 190 ng, no more than 200 ng, no more than 225 ng, no more than 250 ng, no more than 275 ng, no more than 300 ng, no more than 325 ng, no more than 350 ng, no more than 375 ng, no more than 400 ng, no more than 425 ng, no more than 450 ng, no more than 475 ng, no more than 500 ng, no more than 550 ng, no more than 600 ng, no more than 650 ng, no more than 700 ng, no more than 750 ng, no more than 800 ng, no more than 850 ng, no more than 900 ng, no more than 950 ng, no more than 1,000 ng, no more than 1,200 ng, no more than 1,500 ng, no more than 2,000 ng, no more than 3,500 ng, no more than 5,000 ng.

The dosage of payload, silica material, cytokine protein, inflammasome, interleukin, adjuvant, pharmaceutical composition, therapeutic, prodrug, and other equivalents of the present invention can be no more than 1 μg, no more than 2 μg, no more than 3 μg, no more than 4 μg, no more than 5 μg, no more than 6 μg, no more than 7 μg, no more than 8 μg, no more than 9 μg, no more than 10 μg, no more than 11 μg, no more than 12 μg, no more than 13 μg, no more than 14 μg, no more than 15 μg, no more than 16 μg, no more than 17 μg, no more than 18 μg, no more than 19 μg, no more than 20 μg, no more than 21 μg, no more than 22 μg, no more than 23 μg, no more than 24 μg, no more than 25 μg, no more than 30 μg, no more than 32 μg, no more than 35 μg, no more than 37 μg, no more than 40 μg, no more than 42 μg, no more than 45 μg, no more than 48 μg, no more than 50 μg, no more than 55 μg, no more than 60 μg, no more than 65 μg, no more than 70 μg, no more than 75 μg, no more than 80 μg, no more than 85 μg, no more than 90 μg, no more than 95 μg, no more than 100 μg, no more than 110 μg, no more than 120 μg, no more than 130 μg, no more than 140 μg, no more than 150 μg, no more than 160 μg, no more than 170 μg, no more than 180 μg, no more than 190 μg, no more than 200 μg, no more than 215 μg, no more than 230 μg, no more than 250 μg, no more than 265 μg, no more than 275 μg, no more than 300 μg, no more than 325 μg, no more than 350 μg, no more than 375 μg, no more than 400 μg, no more than 425 μg, no more than 450 μg, no more than 475 μg, no more than 500 μg, no more than 550 μg, no more than 600 μg, no more than 650 μg, no more than 700 μg, no more than 750 μg, no more than 800 μg, no more than 850 μg, no more than 900 μg, no more than 950 μg, no more than 1 mg, no more than 1.5 mg, no more than 2 mg, no more than 2.5 mg, no more than 3 mg, no more than 3.5 mg, no more than 4 mg, no more than 4.5 mg, no more than 5 mg, no more than 5.5 mg, no more than 6 mg, no more than 6.5 mg, no more than 7 mg, no more than 7.5 mg, no more than 8 mg, no more than 8.5 mg, no more than 9 mg, no more than 9.5 mg, no more than 10 mg, no more than 11 mg, no more than 12 mg, no more than 13 mg, no more than 14 mg, no more than 15 mg, no more than 16 mg, no more than 17 mg, no more than 18 mg, no more than 19 mg, no more than 20 mg, no more than 22 mg, no more than 24 mg, no more than 25 mg, no more than 27 mg, no more than 29 mg, no more than 30 mg, no more than 33 mg, no more than 35 mg, no more than 37 mg, no more than 40 mg, no more than 43 mg, no more than 45 mg, no more than 48 mg, no more than 50 mg, no more than 55 mg, no more than 60 mg, no more than 65 mg, no more than 70 mg, no more than 75 mg, no more than 80 mg, no more than 85 mg, no more than 90 mg, no more than 95 mg, no more than 100 mg, no more than 110 mg, no more than 120 mg, no more than 130 mg, no more than 140 mg, no more than 150 mg, no more than 160 mg, no more than 170 mg, no more than 180 mg, no more than 190 mg, no more than 200 mg, no more than 220 mg, no more than 240 mg, no more than 250 mg, no more than 275 mg, no more than 300 mg, no more than 325 mg, no more than 350 mg, no more than 375 mg, no more than 400 mg, no more than 425 mg, no more than 450 mg, no more than 475 mg, no more than 500 mg, no more than 550 mg, no more than 600 mg, no more than 650 mg, no more than 700 mg, no more than 750 mg, no more than 800 mg, no more than 850 mg, no more than 900 mg, no more than 950 mg, no more than 1 g, no more than 1.5 g, no more than 2 g, no more than 2.5 g, no more than 3 g, no more than 3.5 g, no more than 4 g, no more than 4.5 g, no more than 5 g, no more than 5.5 g, no more than 6 g, no more than 6.5 g, no more than 7 g, no more than 7.5 g, no more than 8 g, no more than 8.5 g, no more than 9 g, no more than 9.5 g, no more than 10 g, no more than 11 g, no more than 12 g, no more than 13 g, no more than 14 g, no more than 15 g, no more than 16 g, no more than 17 g, no more than 18 g, no more than 19 g, no more than 20 g, no more than 22 g, no more than 24 g, no more than 25 g, no more than 27 g, no more than 28 g, no more than 30 g, no more than 33 g, no more than 35 g, no more than 38 g, no more than 40 g, no more than 43 g, no more than 45 g, no more than 48 g, no more than 50 g, no more than 55 g, no more than 60 g, no more than 65 g, no more than 70 g, no more than 75 g, no more than 80 g, no more than 85 g, no more than 90 g, no more than 95 g, no more than 100 g.

In an embodiment, treatment with a compound disclosed herein reduces inflammation (e.g., signs and symptoms) by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein reduces inflammation (e.g., signs and symptoms) by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein reduces inflammation (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases tumor proliferation (e.g., size and/or number of tumors) by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases tumor proliferation (e.g., size and/or number of tumors) by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases tumor proliferation (e.g., size and/or number of tumors) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases tumor circumference by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases tumor circumference by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases tumor circumference by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases tumor diameter by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases tumor diameter by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases tumor diameter by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases tumor volume by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases tumor volume by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases tumor volume by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases tumor mass by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases tumor mass by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases tumor mass by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases lesion proliferation (e.g., size and/or number) by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases lesion proliferation (e.g., size and/or number) by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases lesion proliferation (e.g., size and/or number) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases lesion diameter by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases lesion diameter by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases lesion diameter by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases lesion size by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases lesion size by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases lesion size by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases lesion mass by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases lesion mass by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases lesion mass by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases sebaceous cyst circumference by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases sebaceous cyst circumference by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases sebaceous cyst circumference by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases sebaceous cyst diameter by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases sebaceous cyst diameter by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases sebaceous cyst diameter by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases sebaceous cyst volume by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases sebaceous cyst volume by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases sebaceous cyst volume by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

In an embodiment, treatment with a compound disclosed herein decreases sebaceous cyst mass by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In an embodiment, treatment with a compound disclosed herein decreases sebaceous cyst mass by, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%.

In an embodiment, treatment with a compound disclosed herein decreases sebaceous cyst mass by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, gender, and body weight, and disease severity.

The total effective dose of the compositions disclosed herein may be administered to a patient in a single dose or may be administered for a long period of time in multiple doses according to a fractionated treatment protocol. In the pharmaceutical composition disclosed herein, the content of active ingredient may vary depending on the disease severity. However, the effective dose of the compositions disclosed are determined considering various factors including patient's age, body weight, health conditions, gender, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

In various embodiments, a formulation can include one or more preservatives and/or additives known in the art. Similarly, a formulation can further be formulated, without limitation, into any of various known delivery formulations. For example, in an embodiment, a formulation can include, surfactants, adjuvant, biodegradable polymers, hydrogels, etc., such optional components, their chemical and functional characteristics are known in the art. Similarly known in the art are formulations that facilitate rapid, sustained or delayed release of the bioactive agents after administration. A formulation as described can be produced to include these or other formulation components known in the art.

The composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the bioactive agents in formulations described herein can, without limitation, be administered to patients throughout an extended time period, such as chronic administration for a chronic condition.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto injectors, safety syringes, injection pumps, infusion pumps, glass prefilled syringes, plastic prefilled syringes and needle free injectors syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, California, U.S.A. Administration by injection may be, without limitation intravenous, intramuscular, intraperitoneal, or subcutaneous, as appropriate. Administrations by non-injection route may be, without limitation, nasal, oral, ocular, cochlear, dermal, or pulmonary, as appropriate. The above injection device may be used in conjunction with catheters, cannulas, ports, shunts and the like.

In certain embodiments, kits can include one or more single or multi-chambered syringes (e.g., liquid syringes and lyosyringes) for administering one or more formulations described herein. In various embodiments, the kit can comprise formulation components for parenteral, subcutaneous, intramuscular or IV administration, sealed in a vial under partial vacuum in a form ready for loading into a syringe and administration to a subject. In this regard, the composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide compositions in accordance therewith. In various embodiment the kits can contain a lyophilate and a sterile diluent for reconstituting the lyophilate.

Also described herein, are methods for treating a subject in need of therapy, comprising administering to the subject an effective amount of a formulation as described herein. The therapeutically effective amount or dose of a formulation will depend on the disease or condition of the subject and actual clinical setting.

In an embodiment, a formulation as described herein can be administered by any suitable route, specifically by parenteral (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary, without limitation, with the composition used for therapy, the purpose of the therapy, and the subject being treated. Single or multiple administrations can be carried out, without limitation, the dose level and pattern being selected by the treating physician. Suitable dosage formulations and methods of administering the agents are known in the art.

The formulations as described herein can be used in the manufacture of medicaments and for the treatment of humans and other animals by administration in accordance with conventional procedures.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, or 14.

In an embodiment, the pH of the pharmaceutical formulation is from about 3 to about 9, about 4 to about 9, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10, about 7 to about 11, about 8 to about 9, about 8 to about 10, about 8 to about 11, about 8 to about 12, about 8 to about 13, about 8 to about 14, about 3 to about 10, about 3 to about 11, about 3 to about 12, about 3 to about 13, about 3 to about 14, about 5 to about 10, about 5 to about 12, about 5 to about 13, about 5 to about 14, about 6 to about 10, about 6 to about 11, about 6 to about 12, about 6 to about 13, about 6 to about 14, about 7 to about 12, about 7 to about 13, about 7 to about 14.

### EXAMPLES

The compositions and methods described herein will be further understood by reference to the following examples, which are intended to be purely exemplary. The compositions and methods described herein are not limited in scope by the exemplified embodiments, which are intended as illustrations of single aspects only. Any methods that are functionally equivalent are within the scope of the invention. Various modifications of the compositions and methods described herein in addition to those expressly described herein will become apparent to those skilled in the art from the foregoing description and accompanying figures. Such modifications fall within the scope of the invention.

### Example 1

### Synthesis of Mesoporous Silica Rods

Methods of producing Mesoporous Silica Rods (MSRs) are known in the art. The size, morphology, pore size, and pore structure of MSRs can be rationally designed and the synthesis process can be freely controlled. Particle size and shape can also influence their blood circulation, cellular uptake and tumor penetration, being determinant parameters to achieve therapeutic effects. Regarding particle size, a diameter range from 50 to 300 nm can favor an optimal cellular uptake, long circulation time, high drug loading and high accumulation in tumors.

In aspects, five ingredients are used in a simple manufacturing process with low cost and minimal complex chemistry.
1) Poly (ethylene glycol)-block-poly(propylene glycol)-block-poly(ethylene glycol), Pluronic® (P123)
2) Water for injection (WFI)
3) Hydrochloric acid 37% acid-fuming
4) Tetraethyl orthosilicate (TEOS)
5) Pure 70% Ethanol

The mesoporous materials can vary in their structural arrangement and pore size. For example, MCM-48 has a cubic arrangement whereas MCM-50 has a lamella-like arrangement. Non-ionic triblock copolymers like alkyl poly(ethylene oxide) (PEO) oligomeric surfactants and poly(alkylene oxide) block copolymers have also been used for synthesis of SBA. The ratio of ethylene oxide to propylene oxide can be varied to achieve a desired symmetry of mesoporous materials: (SBA-11 (cubic), SBA-12 (3-d hexagonal), SBA-15 (hexagonal) and SBA-16 (cubic cage-structured). This is different from MCM in that they possess larger pores of 4.6-30 nm and thicker silica walls. FSM-16, that is, folded sheets of mesoporous materials are another type of mesoporous materials, which can be synthesized using quaternary ammonium surfactant as a template and layered polysilicate kanemite.

### Synthesis/Modification of Mesoporous Silica Rods

Alterations in the process for synthesizing mesoporous silica can result in different shapes, sizes, pore volumes, and surface areas. Applicants have optimized a reproducible and controlled process for making “standard” macroporous mesoporous silica rods (MSRs). Once the standard MSR process and characterization had been established, the process can be tuned to achieve MSRs that are ideal for a specific application. To demonstrate this principle, MSRs were synthesized that were longer and wider with the intent to increase longevity and degrade slower in vivo.

To synthesize mesoporous silica rods that were longer and wide, Applicants adjusted parameters in the optimized standard MSR process. These parameters include slowing the stir speed and decreasing the hydrochloric acid addition. First, P123 was weighed and melted in a Mettler Toledo Reactor Vessel*. Water for injection was then added and mixed at a constant speed of 400 rpm until P123 went into solution. Next, 11 ml of hydrocholoric acid is added followed by tetraethyl orthosilicate. MSRs are formed for 20 hours. Afterwards, the mixing was stopped and temperature was increased for aging the MSRs for 24 hours. Once set, the solution was washed with ethanol through a sieve and the MSRs collected by vacuum filtration and dried. The MSRs were scraped into a porcelain dish and calcinated at 550° C. for five hours. Once cooled, the MSRs were pure, sterile and depyrogenated and were collected in a sterile container for use.

A Mettler Toledo EasyMax 402 System was used which provides precise control over jacket temperature, internal solution temperature, and stirring speed. This piece of equipment is intergrated with iControl software, which monitors technical data in real time and provides batch reports.

Synthesis of the Mesoporous Silica Rods utilized five ingredients to simplify the manufacturing process, reduce cost, and reduce complex chemistry: P123, EasyMax 402, WFI, HCl and TEOS.

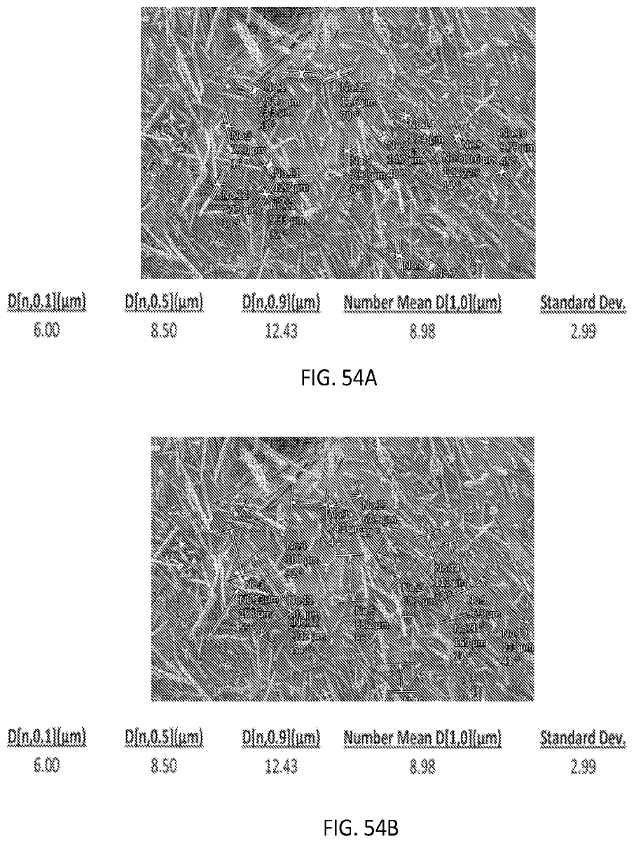

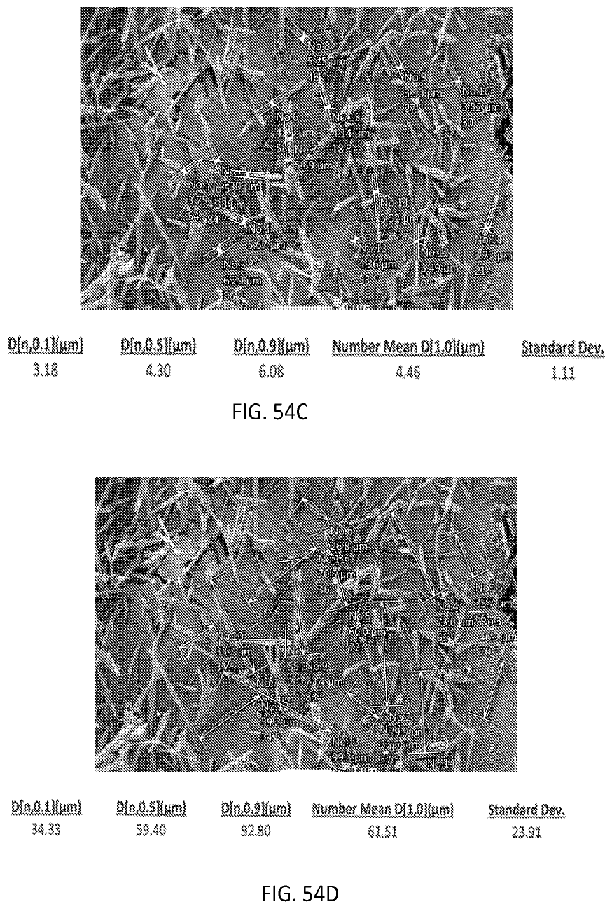

By reducing the stir speed and amount of hydrocholoric acid added during synthesis, MSRs were produced that were longer and wider than Standard MSRs (FIG. 54A-54D). This result also demonstrated that there were no negative effects on the other desired parameters, which included thermogravimetric analysis (TGA), pore volume, and pore size (see below). BET surface area increased as expected with the modified MRSs. In conclusion, this method demonstrated control of certain parameters in process development to produce the desired modified MSR morphology.

DATA SUMMARY

| | | MASS CHANGE | RESIDUAL |
|---|---|---|---|
| | | 25° C. to | MASS at |
| | | 1000° C. | 1000° C. |
| | SAMPLE ID | (%) | (%) |
| | Mesoporous Silica | | |
| Modified | Attivare Run 22 MSR | −2.24 | 97.77 |
| | Lot 20221107 | | |
| Standard | Attivare Run 24 MSR | −2.06 | 97.95 |
| | Lot 20221115 | | |

GAS PHYSISORPTION ISOTHERM DATA SUMMARY

| | | | | |
|---|---|---|---|---|
| | BET | | BJH AVG | |
| | SPECIFIC | BJH PORE | PORE | |
| | SURFACE | VOLUME | DIAMETER | |
| | AREA | 1.7-300 nm | 1.7-300 mm | ADSORBATE |
| SAMPLE ID | (m²/g) | (cm³/g) | (nm) | GAS |

Mesoporous Silica

| | | | | |
|---|---|---|---|---|
| Attivare Run 22 MSR | 929.87 | 1.16 | 4.47 | Nitrogen |
| Lot 20221107 | | | | |
| Attivare Run 23 MSR | 861.54 | 1.03 | 4.23 | |
| Lot 20221107 | | | | |
| Attivare Run 24 MSR | 811.90 | 1.08 | 4.60 | |
| Lot 20221115 | | | | |
| Attivare Run 26 MSR | 966.47 | 1.19 | 4.50 | |
| Lot 20221212 | | | | |

### Vaccine Manufacture

Methods of producing mesoporous silica rods vaccines for humoral response are known in the art. In this example, lyophilized MSRs were suspended in PBS that contained IL-12 (1-20 μg) and gently mixed. The IL-12 was allowed to adsorb to the surface of the MSRs for between one to three hours at 37° C. Next, the contents were pulled into a syringe and injected directly into the tumor. The content and the release profile of the IL-12 can be confirmed using in vitro methods.

### Example 2

### IL-12 Release Content

IL-12 was combined with MSRs as described above. ELISA tests were used to detect the presence of IL-12.

The MSRs were left in PBS solution for seven days. The amount of IL-12 that was released was measured during the course of this seven-day period. Only small amounts of IL-12 were released and could be detected (i.e., less than 3%). Further, there was no cross reactivity found using polyethyleneimine (PEI) L25K alone (1:10 dilution in PBS).

### Example 3

### IL-12 Potency

In-vitro released IL-12 was collected from vaccines and analyzed for potency. The collection protocol was as follows: 6 mL of phosphate-buffered saline (PBS) was introduced and resuspended in a 6× vaccine dose and mixed. This solution was aliquoted into equal 6×1 mL tubes of 1× vaccine doses. The 1× vaccine doses were spun in a centrifuge at 1000 rpm. 700 μl of the resultant was collected and stored at −20° C., then replaced with 700 μl PBS and incubated at 37° C. Post-day seven, 75 μl of mesoporous silica rods and supernatant (SUP) were tested for potency.

The potency results indicated that quantifiably potent IL-12 can only be found on the post-day 7 rods and T-0 (1 μg IL-12 dose w/o PEI). It was also discovered that quantifiably potent IL-12 can be found on post-day 7 rods with PEI alone. However, PEI did not have a significant effect on IL-12 release and potency observed in vitro. Also, 25 k PEI didn't activate HEK cells and was toxic at tested concentrations of 12.5-100 μg/ml in the 5 μg dose.

### Example 4

### Intertumoral IL-12 Delivery

One of the most effective antitumoral cytokines is interleukin-12 (IL-12), which mediates its antitumor activity through stimulation of T, natural killer (NK), and NK T cells through its angiogenic effect. However, systemic IL-12 expression can cause toxicity because of induction of high levels of interferon (IFN)-γ. Strategies allowing local delivery of IL-12 in tumors have been developed, giving rise to high antitumoral efficacy with reduced toxicity.

Due to the toxicity of the material and strong clinical history of antitumoral effects, the idea was conceived to use the MSR biomaterial as an intertumoral delivery vehicle. The concept anticipated the longer-term delivery of the cytokine, potential increased immune response created by the chemistry and configuration of the biomaterial.

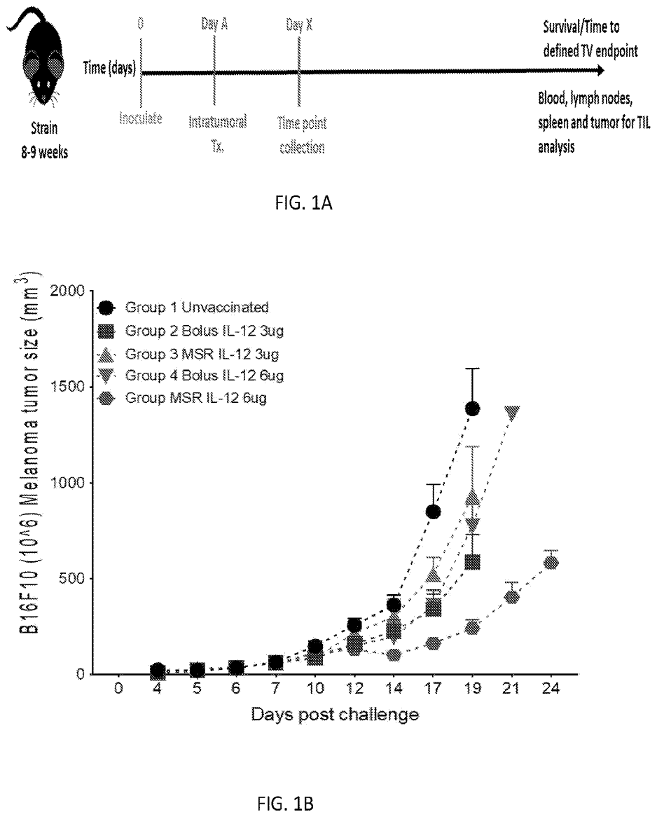

The formulation was tested in B16F10 Melanoma Mouse Model and CT26 colorectal cancer model. FIG. 1A depicts a Melanoma Mouse Model B16F10 for comparing survival times of control and treated mice. Details are summarized as follows:
Mice: female C57/BL6 8 weeks old
Intratumoral vaccine injections via 23G needle by using vaccines containing 6 μg and 20 μg IL-12 in primary tumor (+/−50 mm³)
Intra-flank subcutaneous (s.c.) of B16F10 (10\hat{ }6/mouse) via 29G needle on two flanks
Randomization when tumor reaches about 50 mm³
Tumor burden: three times a week tumor measurement via caliper.
Weight record three times a week
Take down n=3 animals on day 7 post immunization.
TIL analysis in spleen and blood by Flow Cytometry on day 7
Tumors, Implant site, LNs collected at endpoint for histological analysis

### Pilot Studies

The combination treatment (MSR+IL-12) groups have shown better tumor regression and survival (6 μg).

### Example 5

### IL-12 MSR Therapy in Abscopal B16F10 Melanoma Mouse Model

Female mice (C57/BL6-8 weeks old) were administered intratumoral vaccine injections via a 23-gauge needle containing 6 μg and 20 μg of IL-12 in primary tumor (+/−50 mm³ tumor size).

Intra-flank subcutaneous injections of B16F10 were performed via a 29-gauge needle on two flanks.

Tumor burden was measured three times a week via caliper.

Mouse weight was recorded three times per week.

Tumor-infiltrating lymphocyte (TIL) analysis was performed in spleen and blood by flow cytometry on day 7 of treatment.

Tumors, implant site, LNs were collected at endpoint for histological analysis.

### Example 6

### Cytokine MSR Therapy in a B16F10 Solid Tumor Mouse Model

Female mice (C57/BL6-8 weeks old) were administered intratumoral vaccine injections via a 21-gauge needle containing 3 μg and 6 μg of cytokine.

Intra-flank subcutaneous injections of B16F10 were performed via a 29-gauge needle.

Tumor burden was measured three times per week via caliper. Weight was recorded three times per week.

Tumor-infiltrating lymphocyte (TIL) analysis in spleen, lymph nodes and blood was performed by flow cytometry and tumors were collected at endpoint for histological analysis.

### Example 7

### CT-26 Colon Carcinoma Tumor Studies

In this study, the therapeutic effects of various doses of IL-12 coated MSR vaccines were administered as an intratumorally treatment for murine CT-26 colon carcinoma tumor. The MSR vaccines were compared to MSR alone and bolus control groups. The study is summarized in Table 1 below.

Mice were inoculated with 0.5*10⁶ CT-26 tumor cells s.c. on day zero. When tumors reach the volume of 50 mm³ (+/−10 mm³), mice were randomized into groups that received IL-12 MSR vaccines at two different doses (6 μg, 20 μg), IL-12 and MSR control by intratumorally injections via 23-gauge needle (50 μL). Tumor dimensions were measured two days a week using a digital caliper (length×width²)/2. Mice weights were recorded before challenge and treatments over the course of the study three times weekly.

Mice were euthanized when the tumor volume was equal to or greater than 1500 mm³ or if weight loss is equal to or greater than 20%. Other causes for euthanasia included ulceration and bleeding of the tumor, lethargy, and cachexia. Blood for sera collection was collected on day six and 21 after immunization for IFNg and IL-12 quantification via ELISA. On day seven, N3 mice were sacrificed to analyze tumor explants. At endpoint, inguinal lymph nodes and tumors were collected and analyzed for tumor infiltrates lymphocytes (TIL) population by histology. Spleens and blood were collected for CTL response by glow cytometry. Survival was recorded.

TABLE 2
AT029 CT-26 MSR IL-12 i.t Assessment Study

| group | treatment | cytokine | Ns | Test article | Tissues | Time Points and Readouts |
|---|---|---|---|---|---|---|
| 1 | Untreated | — | 9 + 3 | 1x PBS | Inguinal lymph | Sera for IL-12 and IFNg D 6, 21 post |
| | | | | | nodes, tumors, | vaccination (IL-12, IFNg ELISA) |
| | | | | | spleens | Spleen and blood: D 7, endpoint (CD3, |
| | | | | | | CD8, CD4, IFNg, Treg, NK cells) |
| | | | | | | Tumors and lymph node for |
| | | | | | | histology: D 7, endpoint (TIL s |
| | | | | | | infiltration) |
| 2 | MSR i.t. | — | 9 + 3 | MSR alone | Inguinal lymph | Sera for IL-12 and IFNg D 6, 21 post |
| | | | | | nodes, tumors, | vaccination (IL-12, IFNg ELISA) |
| | | | | | spleens | Spleen and blood: D 7, endpoint (CD3, |
| | | | | | | CD8, CD4, IFNg, Treg, NK cells) |
| | | | | | | Tumors and lymph node for |
| | | | | | | histology: D 7, endpoint (TIL s |
| | | | | | | infiltration) |
| 3 | MSR, IL-12 | IL-12 | 9 + 3 | (IL-12: 6 μg) | Inguinal lymph | Sera for IL-12 and IFNg D 6, 21 post |
| | (i.t.) - 6 μg | | | (MSR-XX: | nodes, tumors, | vaccination (IL-12, IFNg ELISA) |
| | | | | 1 mg) | spleens | Spleen and blood: D 7, endpoint (CD3, |
| | | | | | | CD8, CD4, IFNg, Treg, NK cells) |
| | | | | | | Tumors and lymph node for |
| | | | | | | histology: D 7, endpoint (TIL s |
| | | | | | | infiltration) |
| 4 | IL-12 (i.t.) - | IL-12 | 9 + 3 | (IL-12: 6 μg) | Inguinal lymph | Sera for IL-12 and IFNg D 6, 21 post |
| | 6 μg | | | | nodes, tumors, | vaccination (IL-12, IFNg ELISA) |
| | | | | | spleens | Spleen and blood: D 7, endpoint (CD3, |
| | | | | | | CD8, CD4, IFNg, Treg, NK cells) |
| | | | | | | Tumors and lymph node for |
| | | | | | | histology: D 7, endpoint (TIL s |
| | | | | | | infiltration) |
| 5 | MSR, IL-12 | IL-12 | 9 + 3 | (IL-12: | Inguinal lymph | Sera for IL-12 and IFNg D 6, 21 post |
| | (i.t.) - 20 μg | | | 20 μg) | nodes, tumors, | vaccination (IL-12, IFNg ELISA) |
| | | | | (MSR-XX: | spleens | Spleen and blood: D 7, endpoint (CD3, |
| | | | | 1 mg) | | CD8, CD4, IFNg, Treg, NK cells) |
| | | | | | | Tumors and lymph node for |
| | | | | | | histology: D 7, endpoint (TIL s |
| | | | | | | infiltration) |
| 6 | IL-12 (i.t.) - | IL-12 | 9 + 3 | (IL-12: | Inguinal lymph | Sera for IL-12 and IFNg D 6, 21 post |
| | 20 μg | | | 20 μg) | nodes, tumors, | vaccination (IL-12, IFNg ELISA) |
| | | | | | spleens | Spleen and blood: D 7, endpoint (CD3, |
| | | | | | | CD8, CD4, IFNg, Treg, NK cells) |
| | | | | | | Tumors and lymph node for |
| | | | | | | histology: D 7, endpoint (TIL s |
| | | | | | | infiltration) |

### Results

Average tumor growth after 3 μg treatments with cytokine displayed a greater than six-fold decrease when compared to unvaccinated mice. In relation to control (IT Cytokine), treatment with 3 μg cytokine showed an approximately two-fold improvement in reduced average tumor growth.

FIG. 1B is a multivariable line graph showing the effect of interleukin-12 (IL-12) as it mediates antitumor activity in a B16F10 melanoma mouse model through stimulation of T, natural killer (NK), and NK T cells through angiogenic effects. Group 1 mice (control) were unvaccinated (circles). Group 2 received a single dose (3 μg) of IL-12. Group 3 were treated with mesoporous silica rods containing IL-12 (3 μg). Group 4 received a single dose (6 μg) of IL-12. The mice treated with mesoporous silica rods containing IL-12 (6 μg) showed the lowest tumor sizes (hexagons).

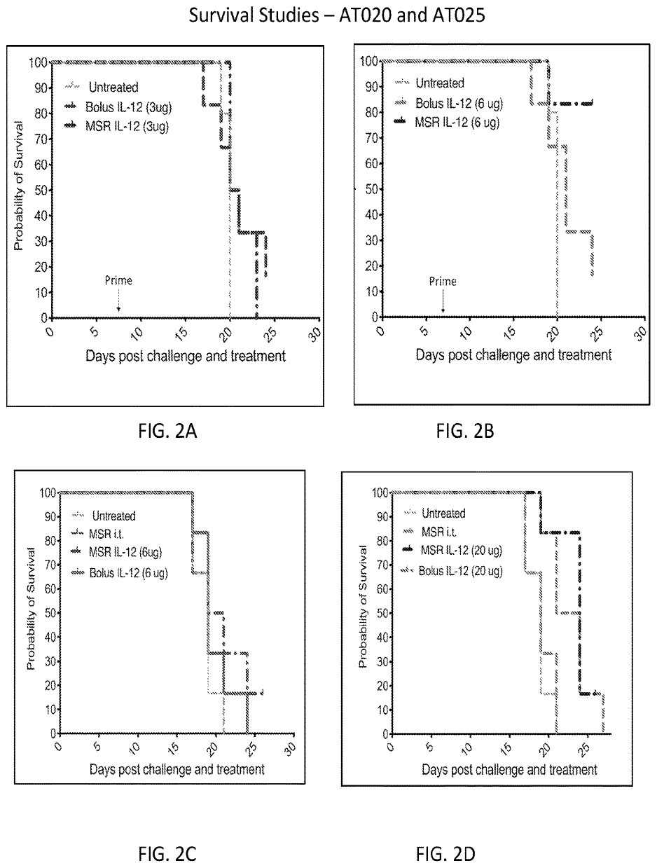

Mice treated with mesoporous silica rods containing IL-12 (6 μg) also showed the greatest probability of survival. FIG. 2A-2D show results of survival studies of murine models. FIG. 2A shows results of mice exposed to a 3 μg bolus of IL-12, a 3 μg dose of MSR IL-12, and an untreated control. FIG. 2B shows results of mice exposed to a 6 μg bolus of IL-12, a 6 μg dose of MSR IL-12, and an untreated control. As with the other studies, the most striking results were observed in mice treated with mesoporous silica rods containing IL-12 (6 μg).

Average tumor growth after 6 μg treatments with cytokine displayed a greater than seven-fold decrease when compared to unvaccinated mice. In relation to control (IT Cytokine), treatment with 6 μg cytokine showed an approximately three-fold improvement in reduced average tumor growth.

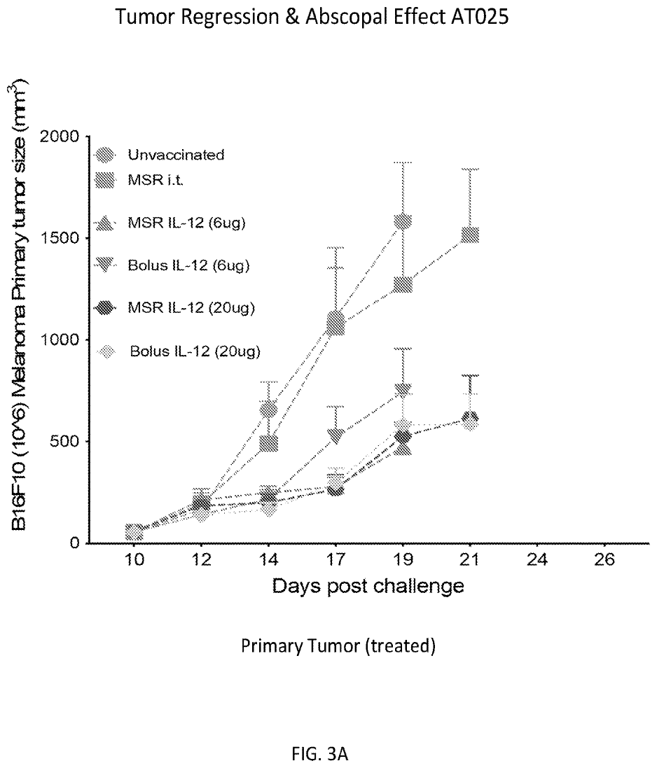

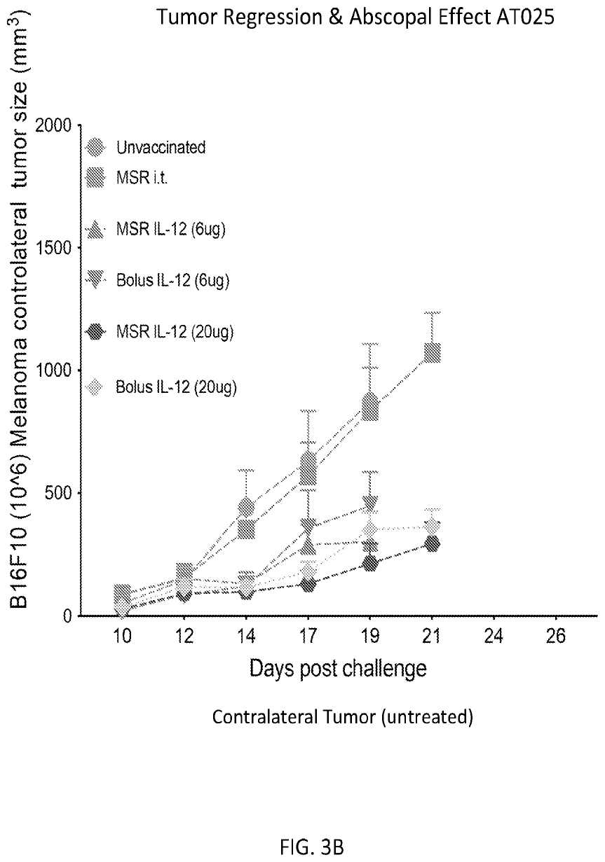

FIGS. 3A and 3B show results of tumor regression and the abscopal effect. As above, the greatest response (i.e., smallest tumor size) was observed in mice treated with mesoporous silica rods containing IL-12 (6 μg). Similar results were observed in contralateral tumors.

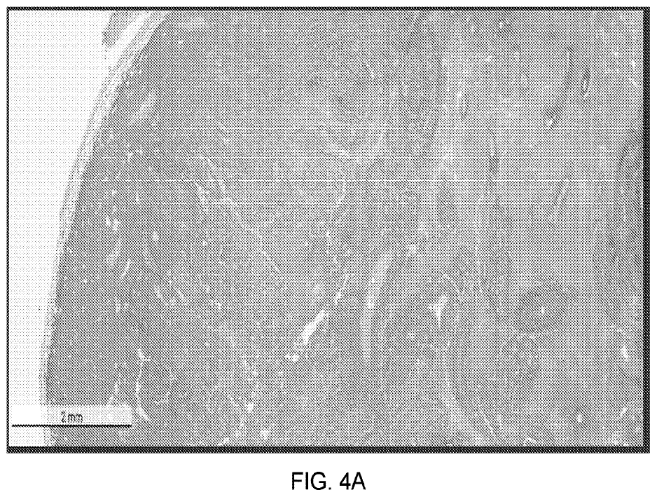

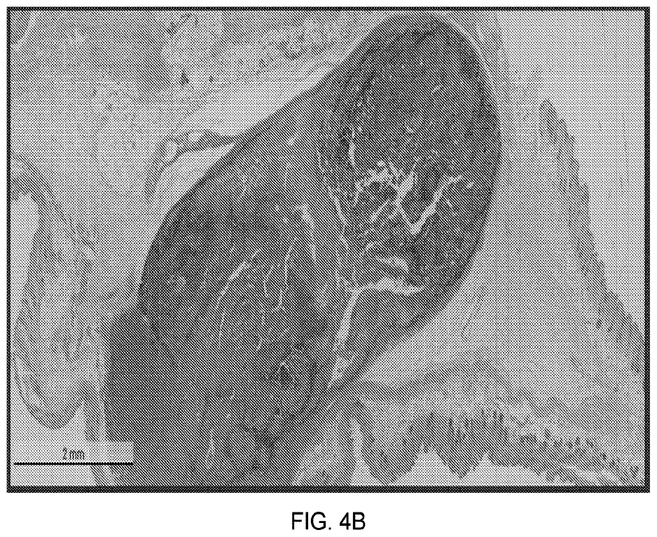

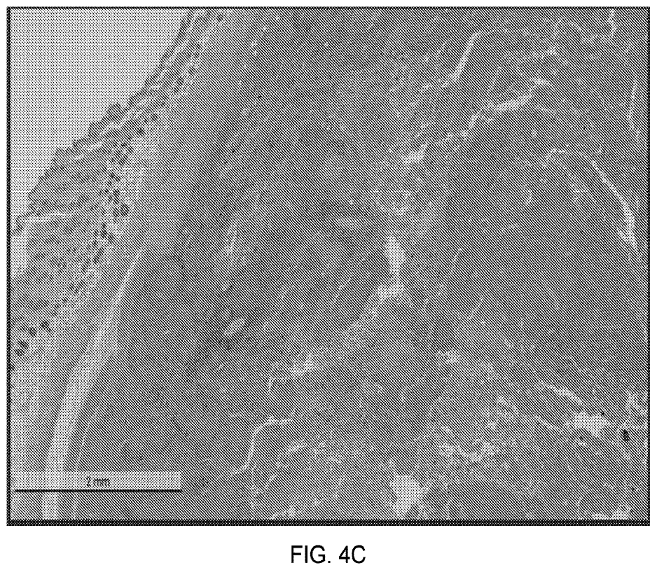

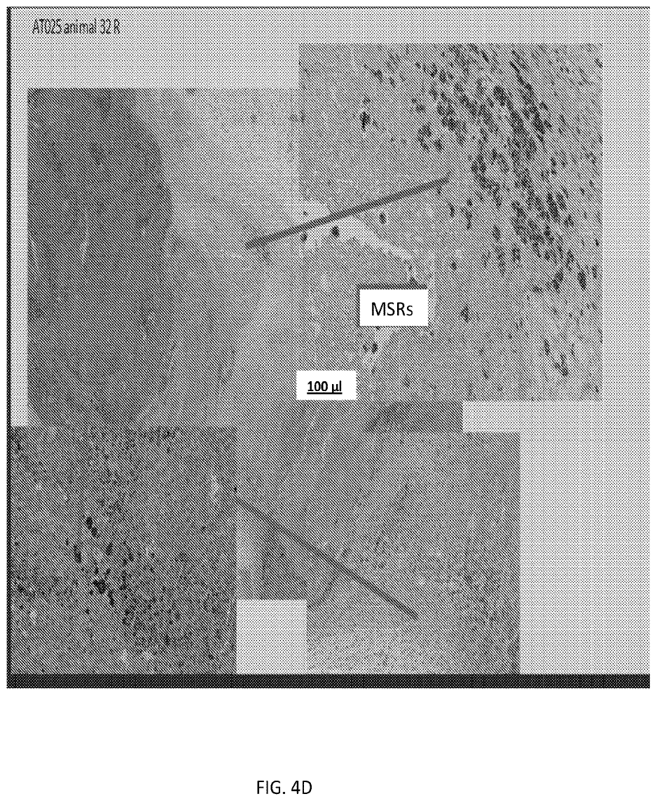

The histology indicates that the IL-12/MSR treatment has a significant impact on the vasculature of the tumor as compared to the other treatment groups. In addition, melanophages (macrophages with melanin from melanocytes) were present in large numbers. FIG. 4A-4D are histological images of a taken from treated and control mice. FIG. 4A shows a histological sample of a 20 μg bolus injection of IL-12. FIG. 4B shows a histological sample of a tumor treated with 20 μg of MSR IL-12. FIG. 4C shows a histological sample of an untreated contralateral tumor. FIG. 4D shows various histological samples wherein mesoporous silica rods (MSRs) were injected into tumor sites. The histology suggests that the IL-12/MSR treatment has a significant impact on the vasculature of the tumor as compared to the other treatment groups. In additional melanophages (Macrophages with Melanin from Melanocytes) were present in large numbers.

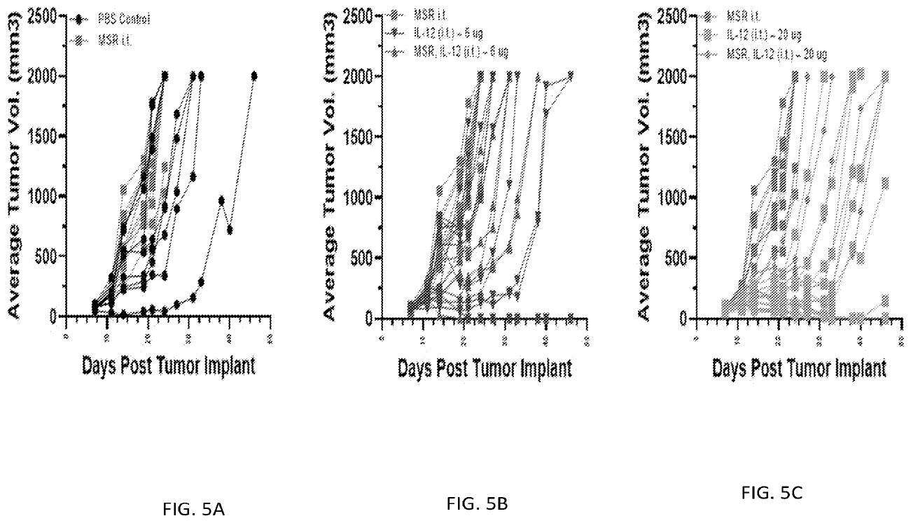

FIG. 5A-5C show results of the CT-26 colon carcinoma tumor study (average tumor volume over time). FIG. 5A shows a comparison of mice treated with MSR compared to control (PBS). FIG. 5B shows results mice treated with MSR compared to mice treated with IL-12 (6 μg) and MSR+IL-12 (6 μg). FIG. 5C shows results of mice treated with MSR compared to mice treated with IL-12 (20 μg) and MSR+IL-12 (20 μg).

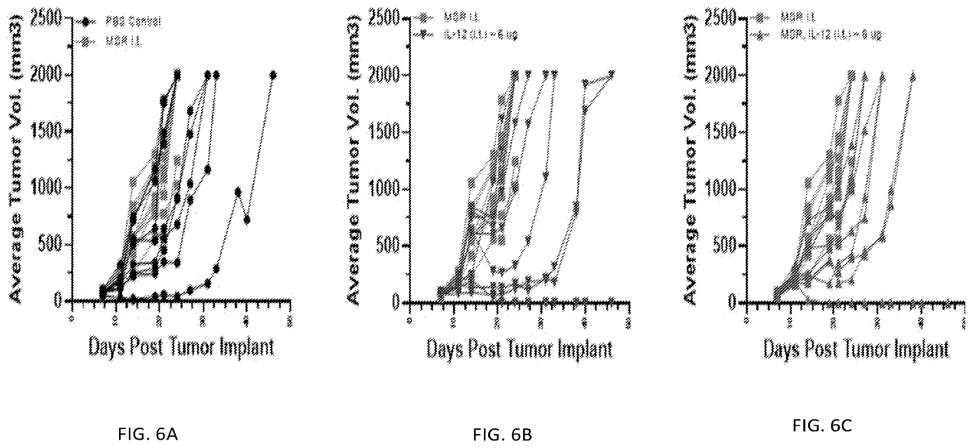

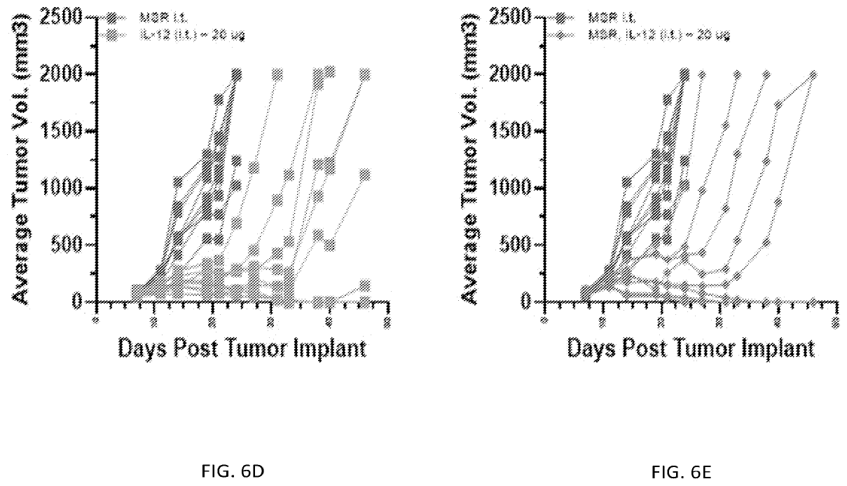

Similarly, FIG. 6A-6E show results of average tumor volume over time. In FIG. 6A, mice treated with MSR compared to control (PBS). FIG. 6B shows mice treated with MSR compared to mice treated with IL-12 (6 μg). FIG. 6C shows mice treated with MSR compared to mice treated with IL-12 (6 μg) and MSR+IL-12 (6 μg). FIG. 6D shows mice treated with MSR compared to mice treated with IL-12 (20 μg). FIG. 6E shows mice treated with MSR compared to mice treated with MSR+IL-12 (20 μg). Complete regression was observed with the following categories:
a) IL-12 (6 μg) i.t; N1 of 9<0 mm³
b) MSR+IL-12 (6 μg) i.t; N1 of 9<0 mm³
c) IL-12 (20 μg) i.t; N2 of 9<0 mm³
d) MSR+IL-12 (20 μg) i.t; N4 of 9<0 mm³

### Example 8

### ATT-02 and ATT-02+ Intratumoral and Perilymphatic Dosing

In this study, mice were inoculated with 1 million cells on left-flank (primary) and 0.25 million cells on right-flank (secondary) with B16F10 tumor cells subcutaneous (s.c.). Mice with tumors ranging from 100 to 150 mm³ (right) and 25 to 50 mm³ (left) were randomized and divided into treatment groups, receiving 50 μl of Att-02 (IL-12 and MSR) and Att-02+ (which includes cytosine guanosine dinucleotide, “CpG”) MSR. Mice either received an intratumoral (i.t.) dose and perilymphatic dose (p.l.) on the same side (larger tumor) or p.l. doses on each flank (2 flanks). All treatments were administered on day 10 post tumor inoculation.

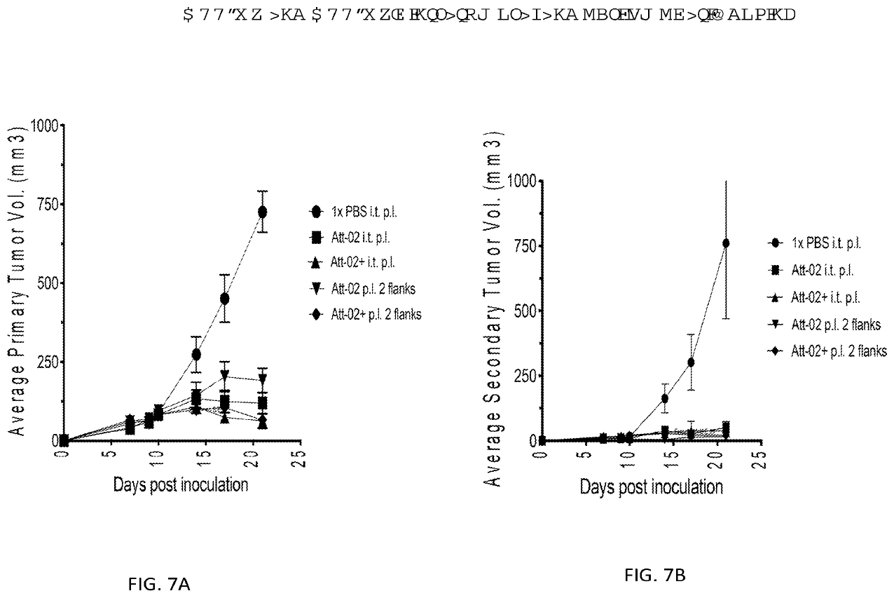

FIGS. 7A and 7B show results of this study (average tumor volume over time). FIG. 7A shows the average primary tumor volume of mice treated with PBS (control) compared to:
ATT-02 it. p.l. (intratumoral and perilymphatic IL-12 and MSR)
ATT-02+ it. p.l. (intratumoral and perilymphatic IL-12 and MSR with CpG)
ATT-02 p.l. 2 flanks (perilymphatic flank IL-12 and MSR)
ATT-02+ it. p.l.2 flanks (perilymphatic flank IL-12 and MSR with CpG)
   Primary tumor growth was lowest in mice that were administered intratumoral (i.t.)+perilymphatic (p.l.). Secondary tumor growth was minimal for all variations. Overall, tumor growth was substantially lower compared to the control.

### Primary Tumor Studies

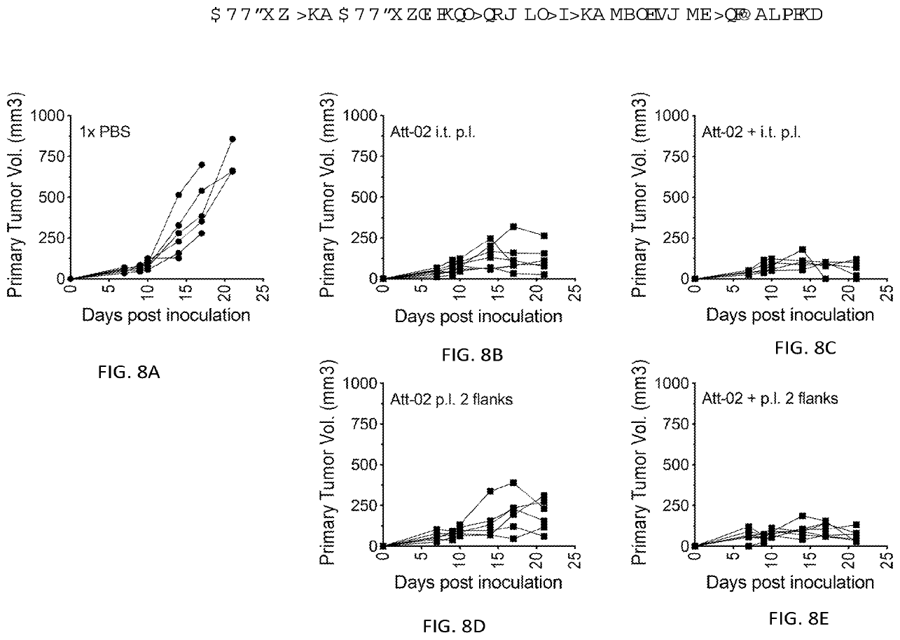

Similarly, FIG. 8A-8E shows results mice treated with each of the above variations. FIG. 8A shows that tumors grew rapidly with PBS (control). FIG. 8B shows primary tumor growth (days post inoculation) after treatment with ATT-02 it. p.l. FIG. 8C shows primary tumor growth (days post inoculation) after treatment with ATT-02+ it. p.l. FIG. 8D shows primary tumor growth (days post inoculation) after treatment with ATT-02 p.l. flanks. FIG. 8E shows primary tumor growth (days post inoculation) after treatment with ATT-02 p.l. 2 flanks. FIG. 8F shows primary tumor growth (days post inoculation) after treatment with ATT-02+p.l. 2 flanks.

### Secondary Tumor Studies

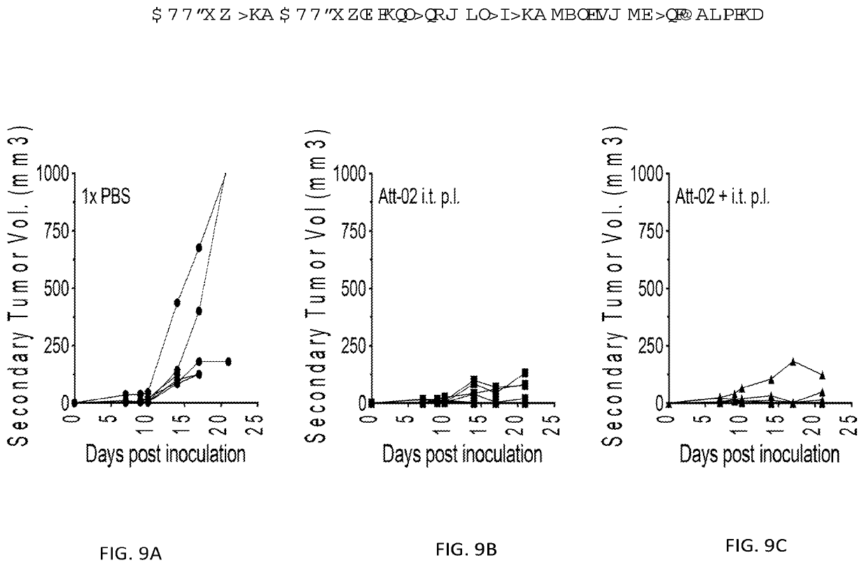

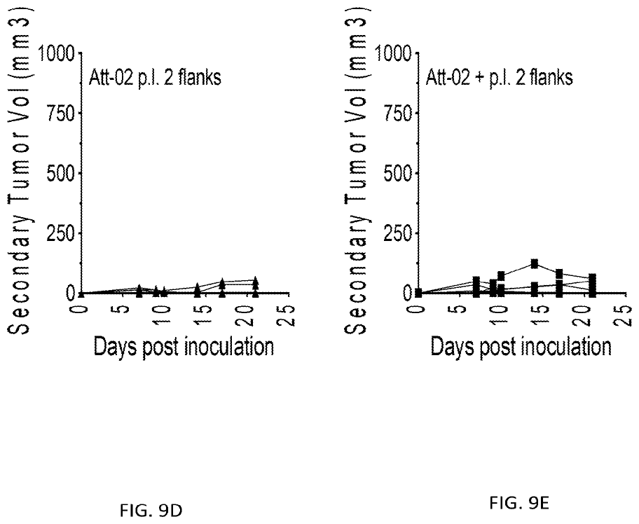

Similarly, FIG. 9A-9E shows results mice treated with each of the above variations. FIG. 9A shows that tumors grew rapidly with PBS (control). FIG. 9B shows primary tumor growth (days post inoculation) after treatment with ATT-02 it. p.l. FIG. 9C shows primary tumor growth (days post inoculation) after treatment with ATT-02+ it. p.l. FIG. 9D shows primary tumor growth (days post inoculation) after treatment with ATT-02 p.l. flanks. FIG. 9E shows primary tumor growth (days post inoculation) after treatment with ATT-02 p.l. 2 flanks. FIG. 9F shows primary tumor growth (days post inoculation) after treatment with ATT-02+p.l. 2 flanks.

### Example 9

IL-12 Studies in Mice Treated with MSRs

In this study, levels of IL-12 levels were measured in mice treated with MSRs and controls.

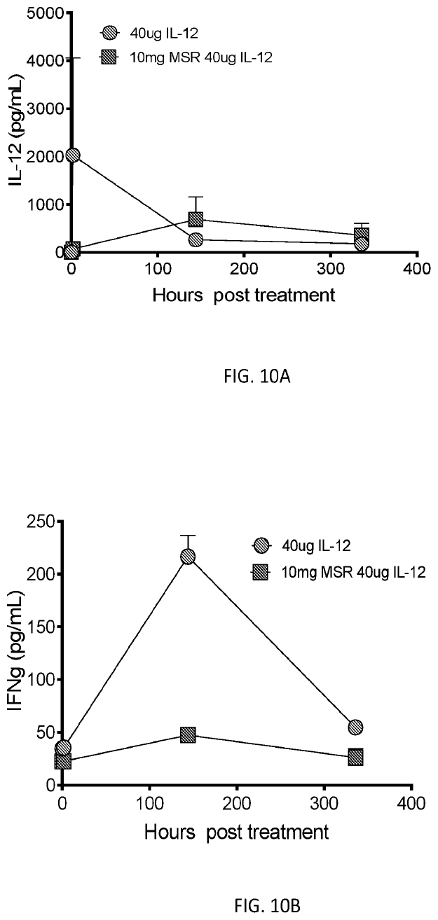

FIG. 10A is a graph comparing IL-12 levels (pg/mL) over time (hours post-treatment). Similarly, FIG. 10B is a graph comparing IFN gamma levels (pg/mL) over time (hours post-treatment).

In summary, IL-12 was detected in mice (n=6) receiving two subcutaneous injections of 5 mg MSR and 20 μg IL-12. A control group received two injections of IL-12 at 20 μg s.s. Serum samples were collected and analyzed 1 hour, 6-, and 12-days post treatment. IL-12 (a) and IFN gamma (b) levels were measured by ELISA kit (Life Technologies). Both cytokines were detected at baseline levels in the MSR IL-12 group.

Comparison of Immune Cell Levels in Mice Treated with MSRs

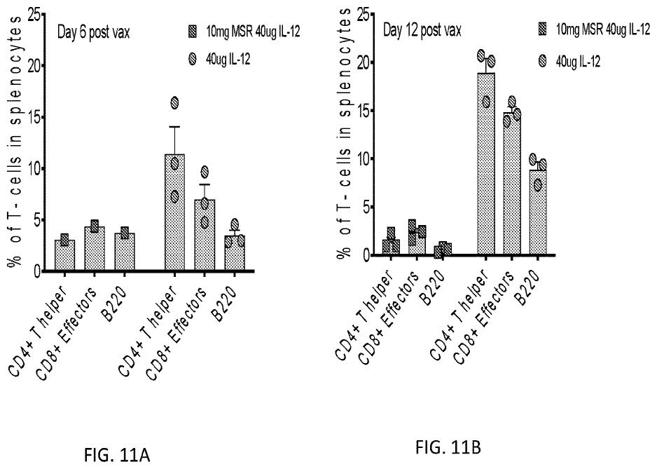

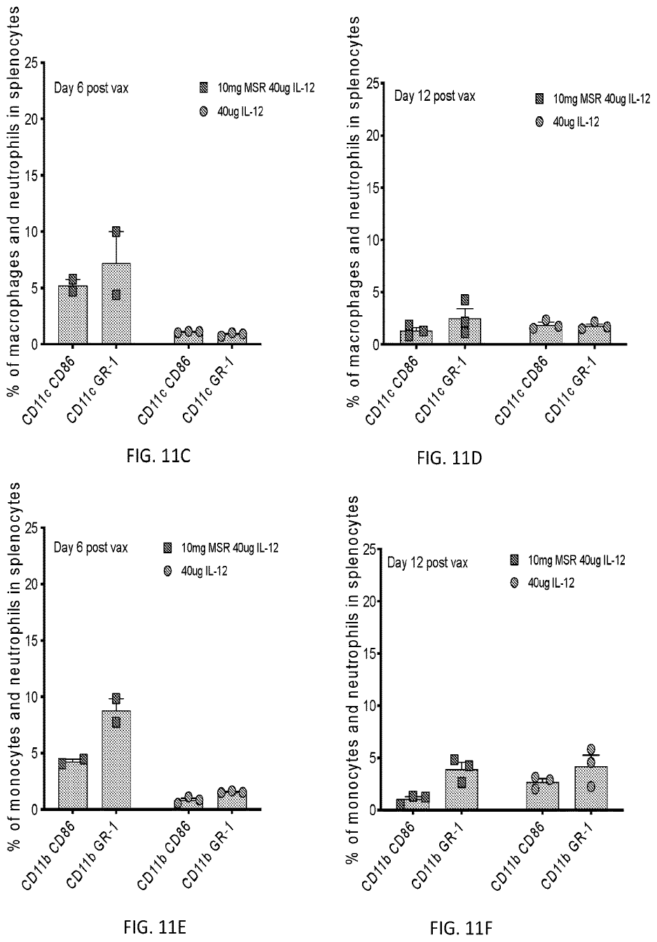

Immune cells activity in splenocytes of mice (n=6) treated with two subcutaneous injections of 5 mg MSR and 20 μg IL-12 as compared to the control group receiving two injections of IL-12 at 20 μg. Splenocytes were isolated and analyzed six days and twelve days post-treatment by flow cytometry. FIG. 11A-11E show the quantification of T-cells, B-cells (11A, 11B); macrophages (11C, 11D), monocytes, and neutrophils (11E, 11F).

### Comparison of IL-12 Levels

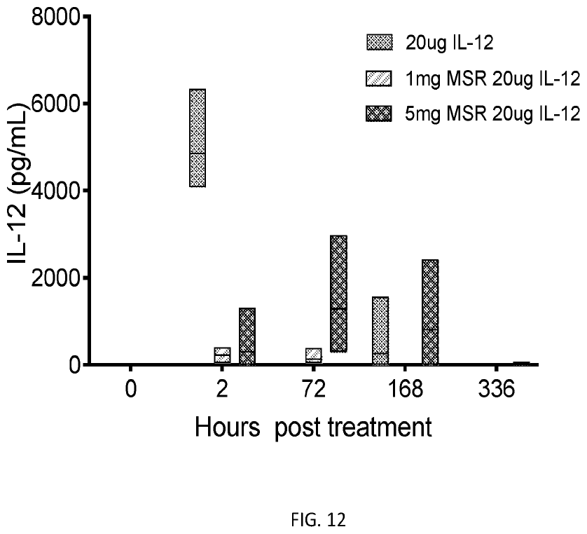

IL-12 detection in mice (n=5) receiving a single subcutaneous injection of 1 mg MSR and 20 μg of IL-12, 5 mg of MSR and 20 μg of IL-12. A control group received a single injection of IL-12 at 20 μg. Serum samples were collected and analyzed 2 hours, 3 days, 7 days and 14 days post-treatment. IL-12 (a) levels were measured by ELISA kit (Life Technologies). FIG. 12 shows IL-12 (pg/mL) versus hours post treatment.

### Blood Analysis

In the next study, blood was analyzed. Results of complete blood counts from blood samples stored in EDTA tubes after 14 days from treatments. White blood cells (a), lymphocytes (b), monocytes (c), and neutrophils (d) were recorded.

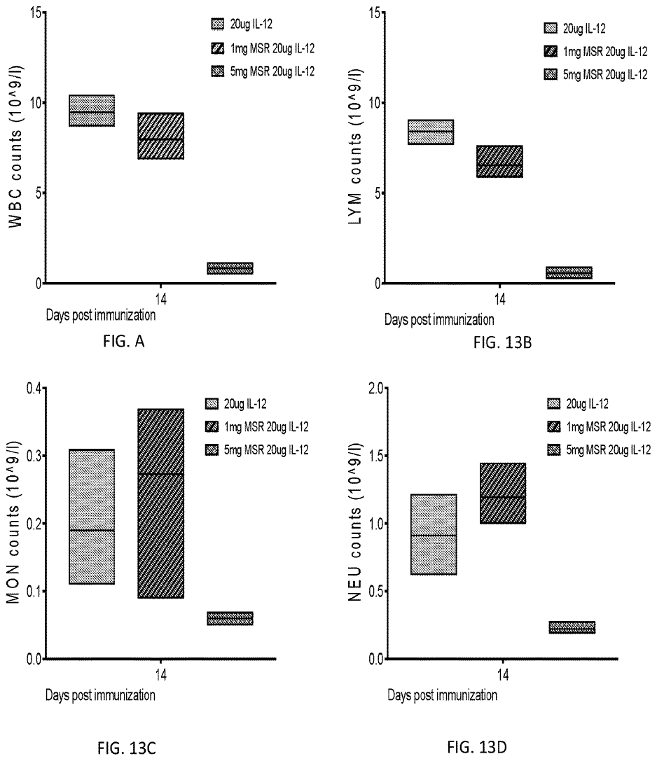

FIG. 13A shows white blood cell (WBC) counts versus days post immunization; FIG. 13B shows lymphocyte (LYM) counts; FIG. 13C shows monocyte (MON) counts; and FIG. 13D shows neutrophil (NEU) counts. The results suggest dose response to MSR of immune cells.

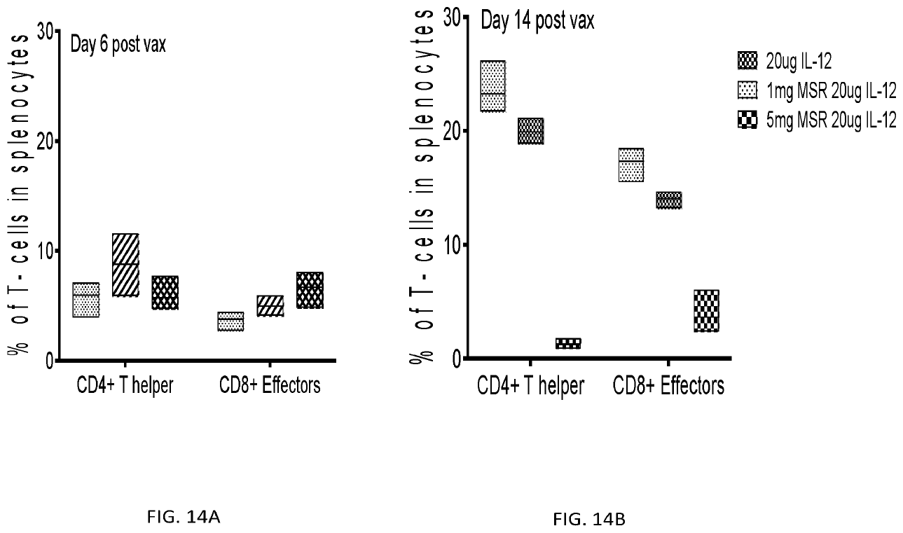

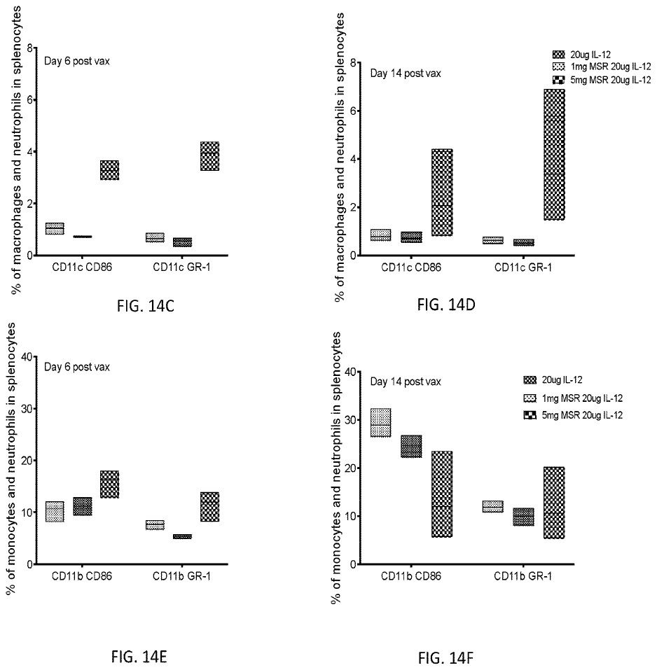

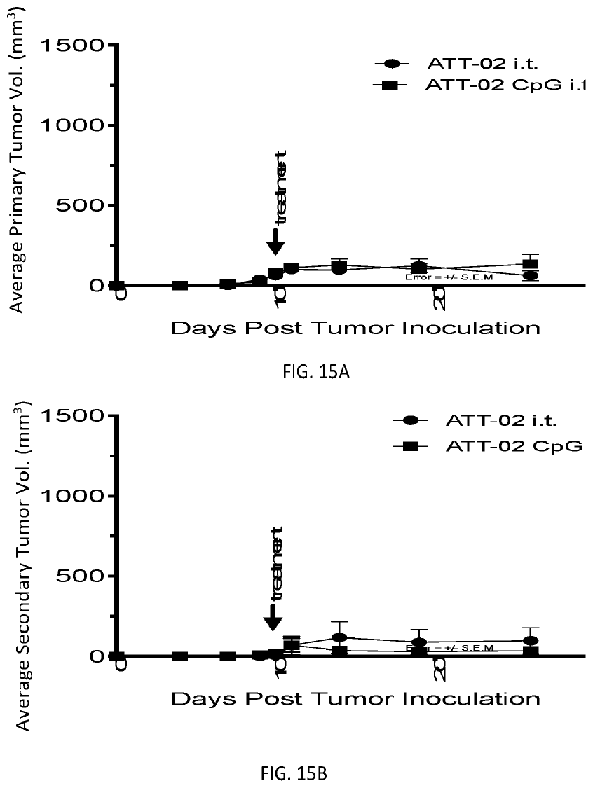

Next, immune cells activity in splenocytes of mice (n=3) treated with a single subcutaneous injection of 1 mg MSR and 20 μg IL-12, 5 mg MSR and 20 μg IL-12 as compared to the control group receiving a single injection of IL-12 at 20 μg. Splenocytes were isolated and analyzed 6 days and 14 days post-treatments by flow cytometry. Shown are the quantification of T-, B-cells (FIG. 14A, 14B); macrophages (FIG. 14C, 14D), monocytes, and neutrophils (FIG. 14E, 14F) immune cells.

### Primary/Secondary Tumor Studies

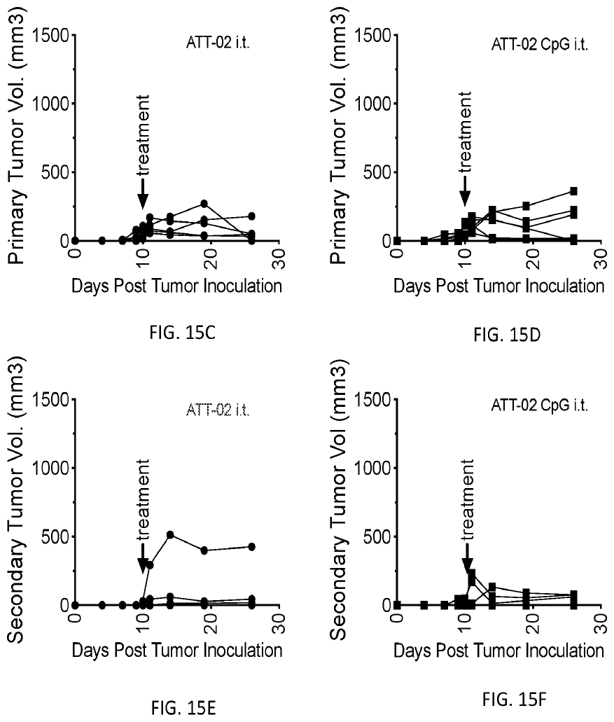

The next study entailed a single treatment of ATT-02 and ATT-02 CpG in the primary tumor. C57/B16 mice (untreated n=10 mice/group, ATT-02 intratumorally n=10 mice/group and ATT-02 CpG intratumorally. n=10 mice/group) were inoculated s.c. with 10\hat{ }6 and 0.25×10\hat{ }6 B16F10 melanoma tumor cells on the left and right flanks, respectively. At day 10, the left flank tumor was treated intratumorally with ATT-02 (1 mg MSR 20 μg IL-12) and ATT-02 CpG (1 mg MSR 20 μg IL-12 and 100 μg CpG). The graphs show averaged tumors (FIG. 15A, 15D) and the individual tumor growth curves (FIG. 15B, 15C, 15E, 15F) for the treated and untreated tumors. The arrows indicate timing of treatments.

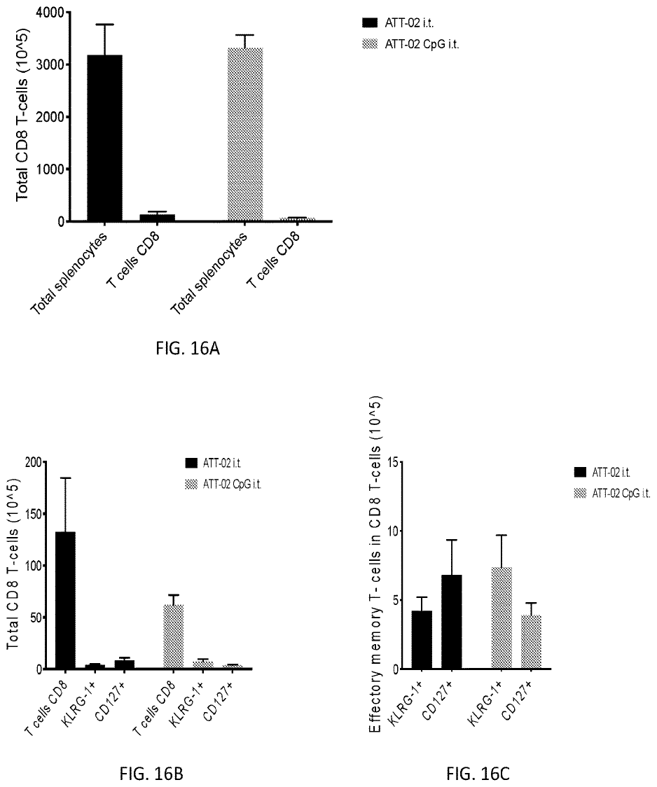

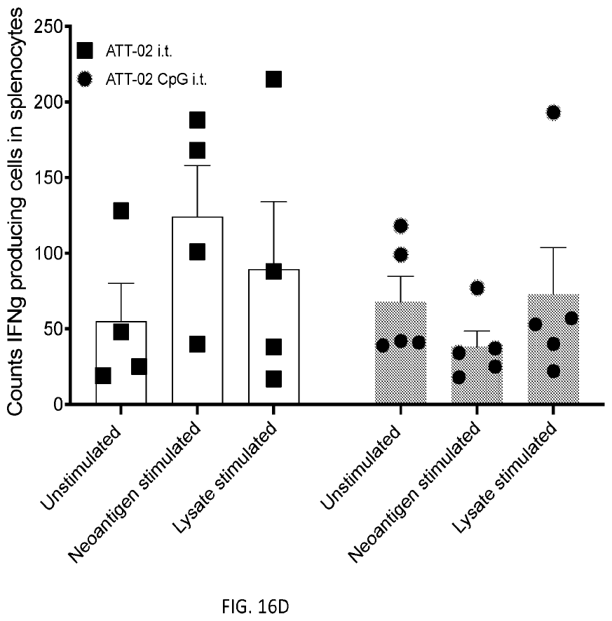

FIG. 16A-16D show the quantification of total splenocytes (a), CD8+ T cells (a,b represent a zoom in the CD8 population), and KLRG-1+ and CD127+ effector memory T-cells (b, c represent a zoom in the KLRG-1 and CD127 population) (mean±s.d.) after ATT-02 and ATT-02 CpG treatments. Spleens were harvested from immunized mice 7 days after treatment. Detection of IFN-γ secreting splenocytes was performed by ELISPOT assays. The splenocytes from ATT-02 and ATT-02 CpG treated mice (n=6) were stimulated with 30 μg/ml of the neoantigen peptides (30 μg each (B16-M27, B16-M40, B16-M27, B16-M47, B16-M48) or 100 μg/ml B16F10 cell lysate. Data shown represent the mean±SD of each group. Unstimulated cells were used as control. Spleens were harvested from immunized mice seven days after treatment.

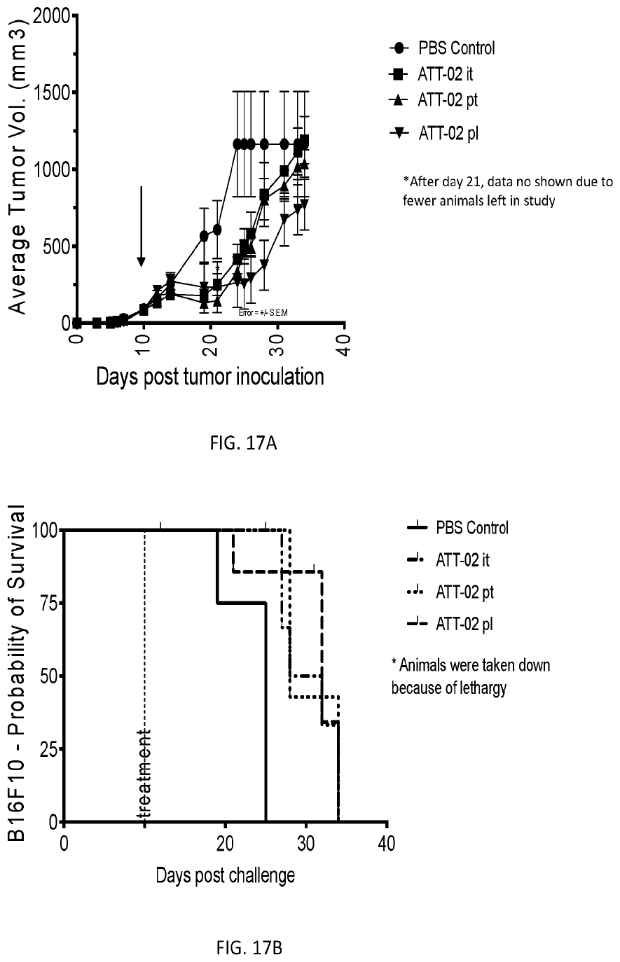

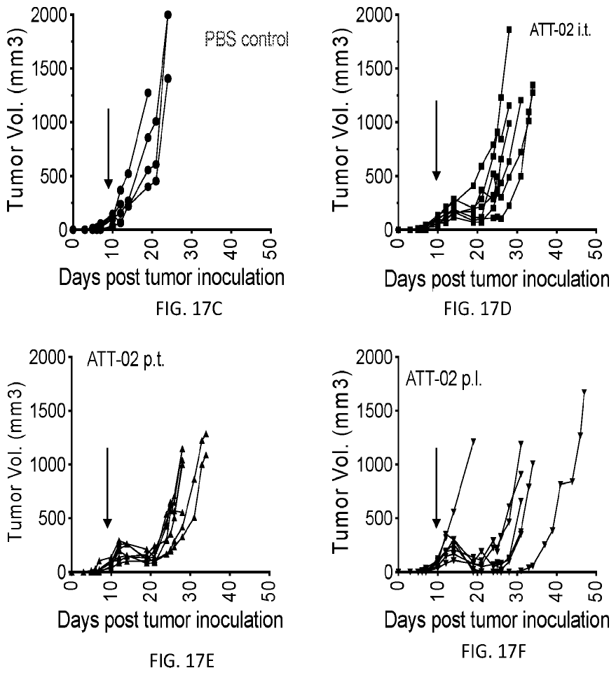

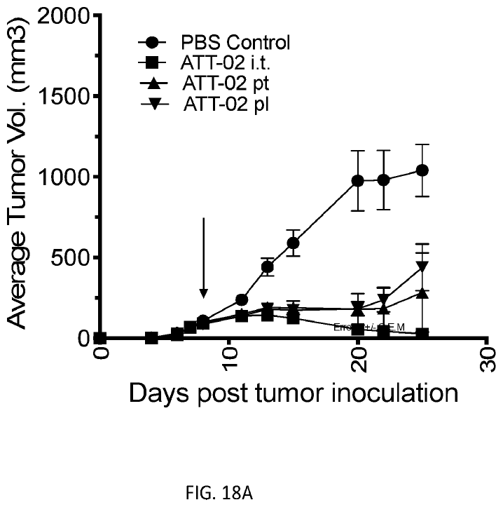

The next figures show the therapeutic effect of a single treatment of ATT-02 intra-tumoral (i.t.), peri-tumoral (p.t.), or peri-lymphatic (p.l.) in B16F10 melanoma model. C57/B16 mice (untreated n=7 mice/group, ATT-02 i.t. n=7 mice/group, ATT-02 p.t., n=7 mice/group ATT-02 p.l.) were inoculated s.c. with 10\hat{ }6 B16F10 melanoma tumor cells on the left flank. At day 10, the left flank tumor was treated i.t., p.t., and p.l. with ATT-02 (1 mg MSR 20 μg IL-12) and PBS was used as control. FIG. 17A, 17C-17F show average tumors and the spider plot for the individual tumor growth curves for the treated and untreated tumors and the overall survival (FIG. 17B). Arrows indicate timing of treatments.

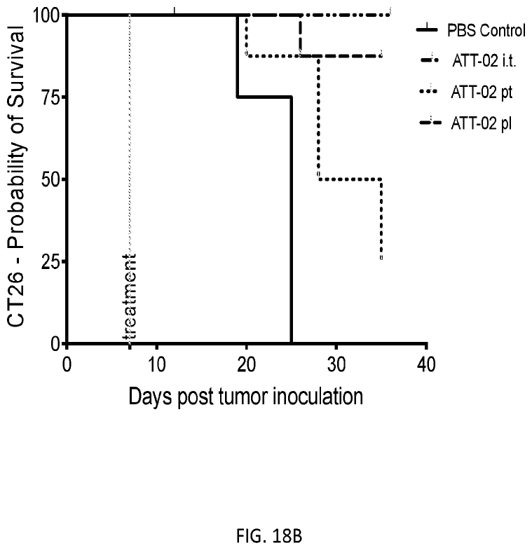

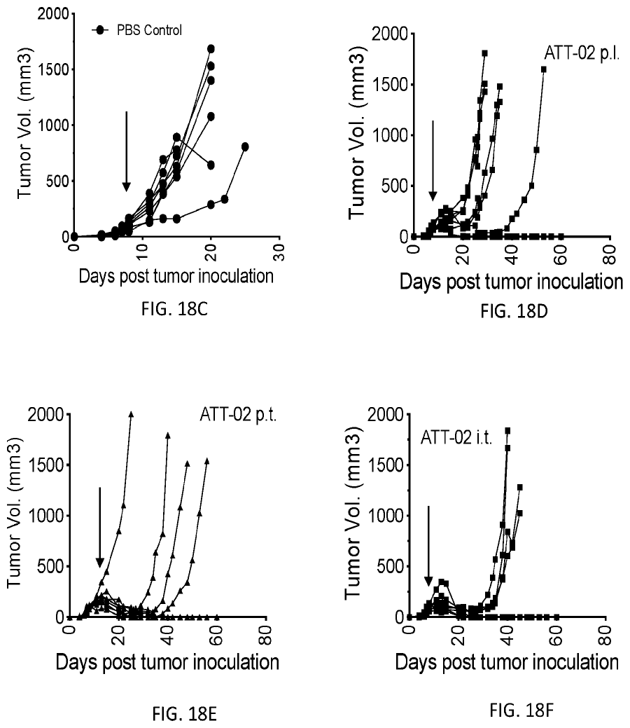

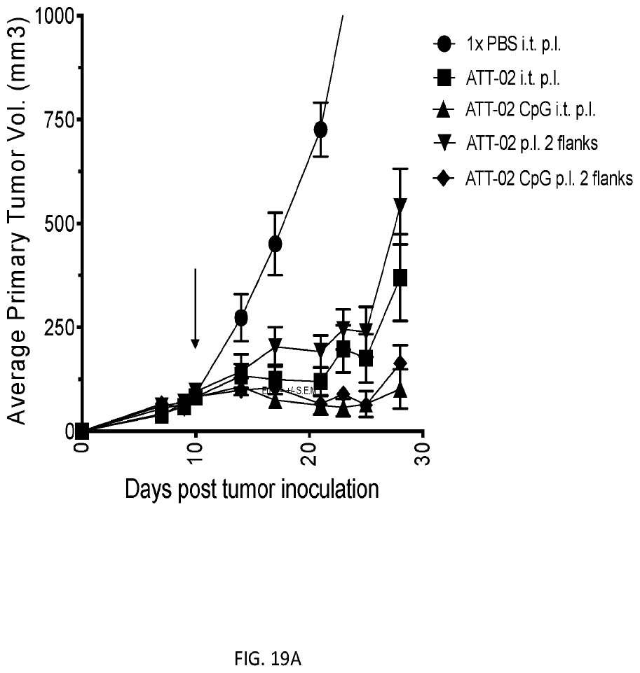

Similarly, the next figures show therapeutic effect of double treatments of ATT-02 and ATT-02 CpG intra-tumoral (i.t.), and peri-lymphatic (p.l.) on both flanks in B16F10 melanoma cancer model. C57 B16 mice (untreated n=7 mice/group, ATT-02 i.t p.l. n=7 mice/group, ATT-02 CpG i.t p.l. n=7 mice/group, ATT-02 p.l., n=7 mice/group ATT-02 CpG p.l.) were inoculated s.c. with 10\hat{ }6 B16F10 melanoma tumor cells on the left flank. At day 10, the left flank tumor was treated intratumorally and perilymphatic with ATT-02 (1 mg MSR 28 μg IL-12) and ATT-02 CpG (1 mg MSR 28 μg IL-12 and 100 μg CpG). FIG. 18A, 18C-18F show averaged tumors and the individual tumor growth curves for the treated tumors and the overall survival (FIG. 18B). Arrows indicate timing of i.t. and p.l. treatments. Arrows indicate timing of treatments.

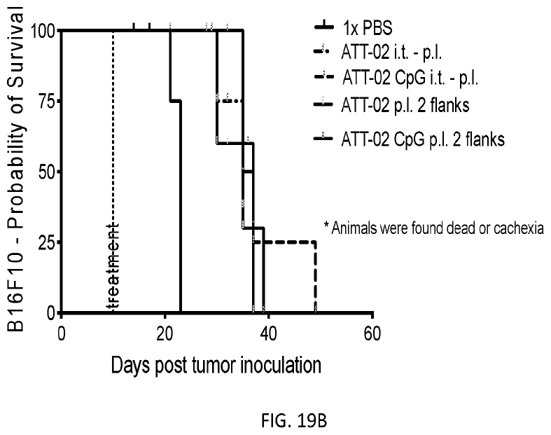

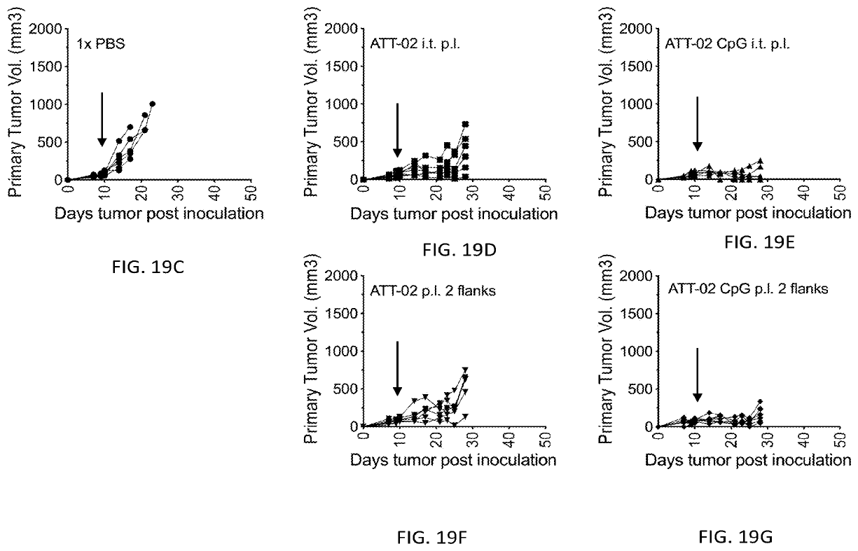

The next figures show the therapeutic effect of double treatments of ATT-02 and ATT-02 CpG intra-tumoral (i.t.), and peri-lymphatic (p.l.) on both flanks in B16F10 melanoma cancer model. C57 BI6 mice (untreated n=7 mice/group, ATT-02 i.t p.l. n=7 mice/group, ATT-02 CpG i.t p.l. n=7 mice/group, ATT-02 p.l., n=7 mice/group ATT-02 CpG p.l.) were inoculated s.c. with 10\hat{ }6 B16F10 melanoma tumor cells on the left flank. At day 10, the left flank tumor was treated intratumorally and perilymphatic with ATT-02 (1 mg MSR 28 μg IL-12) and ATT-02 CpG (1 mg MSR 28 μg IL-12 and 100 μg CpG). FIG. 19A, 19C-19G show is averaged tumors and the individual tumor growth curves for the treated tumors and the overall survival (19B). Arrows indicate timing of i.t. and p.l. treatments.

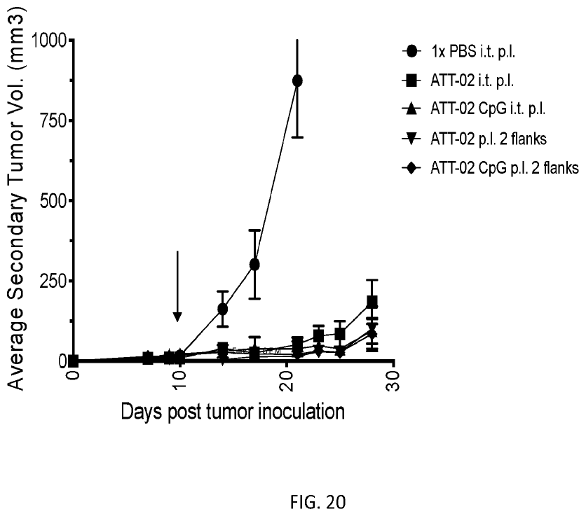

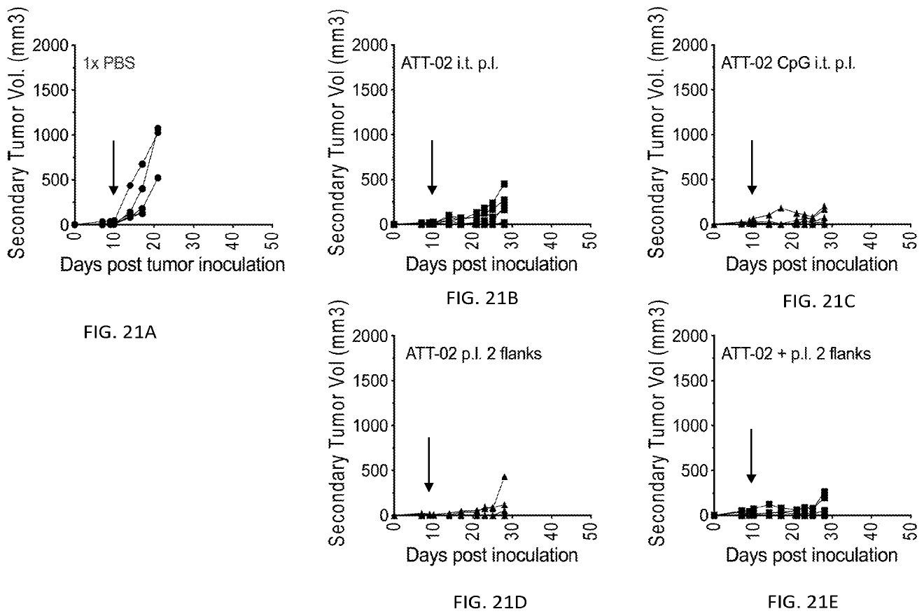

The next figures show the therapeutic effect of double treatments of ATT-02 and ATT-02 CpG intra-tumoral (i.t.), and peri-lymphatic (p.l.) on both flanks in B16F10 melanoma cancer model. C57 BI6 mice (untreated n=7 mice/group, ATT-02 i.t p.l. n=7 mice/group, ATT-02 CpG i.t p.l. n=7 mice/group, ATT-02 p.l., n=7 mice/group ATT-02 CpG p.l.) were inoculated s.c. with 10\hat{ }6 B16F10 melanoma tumor cells on the left flank. At day 10, the left flank tumor was treated intratumorally and perilymphatic with ATT-02 (1 mg MSR 28 μg IL-12) and ATT-02 CpG (1 mg MSR 28 μg IL-12 and 100 μg CpG). FIGS. 20 and 21A-21E show averaged tumors and the individual tumor growth curves for the untreated tumors. Arrows indicate timing of treatments.

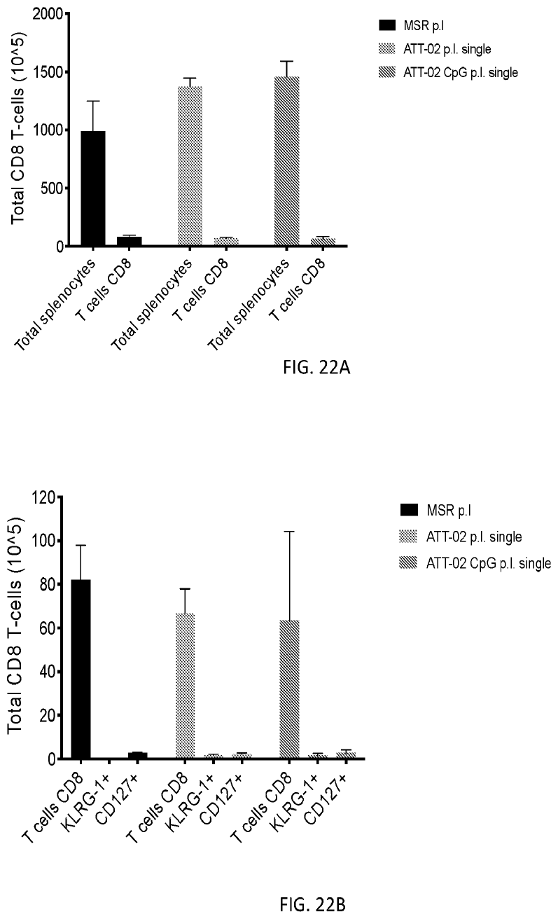

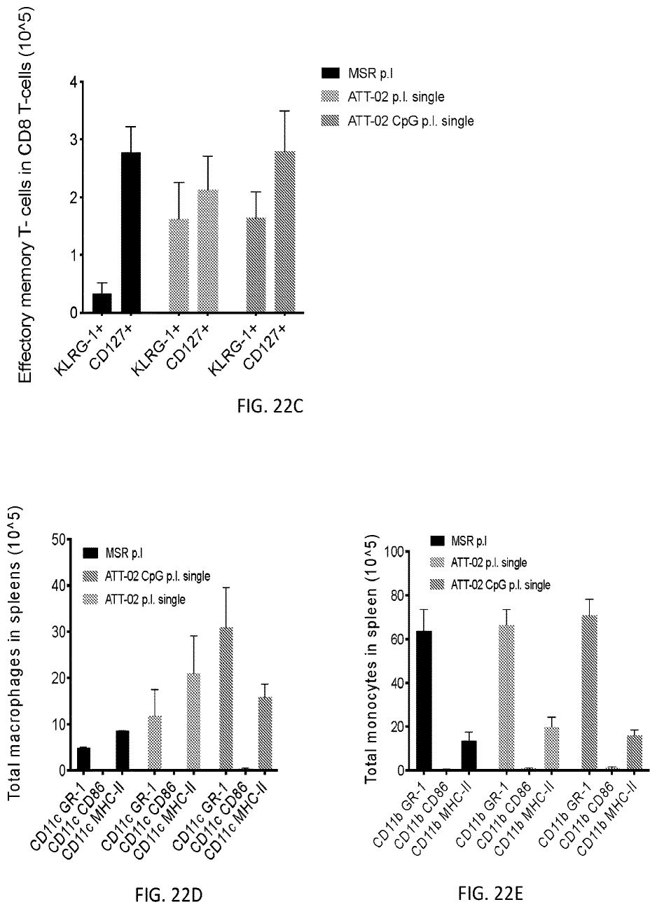

In the next study, mice were inoculated with 1 million cells on left-flank (primary) and 0.25 million cells on right-flank (secondary) with B16F10 tumor cells s.c. FIG. 22A-22E show the quantification of total splenocytes, CD8+ T cells (a, b represent a zoom in the CD8 population), and KLRG-1+ and CD127+ effector memory T-cells (b, c represent a zoom in the KLRG-1 and CD127 population), macrophages, monocytes, and neutrophils (d) immune cells after MSR, ATT-02 and ATT-02 CpG treatments. Spleens were harvested from immunized mice 7 days after treatment. Data shown represent the mean±SD of each group.

### Example 10

Solid Tumor Studies in Mice Treated with MSRs

In the following studies levels of solid tumors were studied in mice treated with MSRs and controls.

Mice were inoculated with 1M CT-26 cells subcutaneously in right flank on day 0. On day 7, mice began treatment with PBS, IL-12 (20 μg, i.t.), PD-1 i.p. every three days, ATT-02 (i.t, 20 μg), MSR (i.t.) and PD-1 (i.p) every three days, ATT-02 (i.t., 20 μg) plus PD-1 (i.p.) every three days. Mice receiving PD-1 was dosed a total of 5 doses PD-1 at 200 μg per mouse.

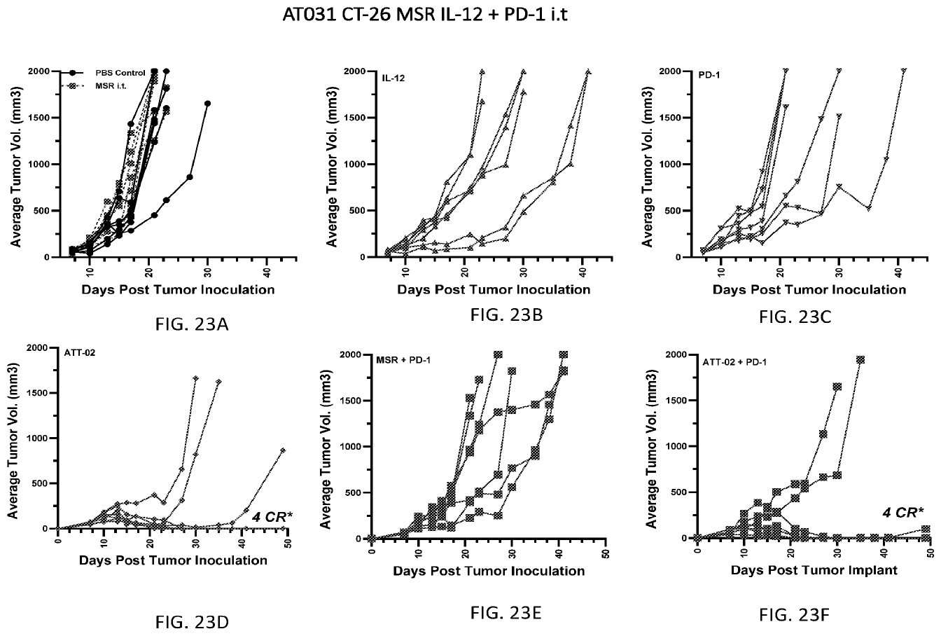

FIG. 23A shows Individual tumor volumes (mm³) of treatment groups PBS control and MSR i.t. FIG. 23B shows individual tumor volumes (mm³) of IL-12 treatment. FIG. 23C shows the individual tumor volumes (mm³) of treatment PD-1. FIG. 23D shows the individual tumor volumes (mm³) of treatment group ATT-02. FIG. 23E shows individual tumor volumes (mm³) of treatment group MSR plus anti PD-1. FIG. 23F shows individual tumor volumes (mm³) of treatment group ATT-02 plus anti PD-1.

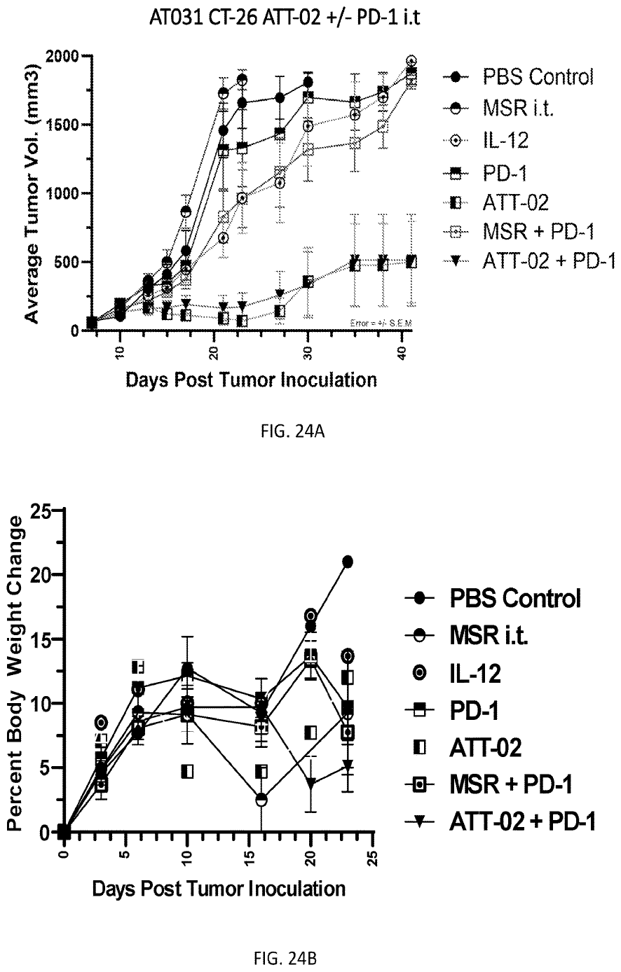

Next, mice were inoculated with 1M CT-26 cells subcutaneously in right flank on day 0. On day seven, mice began treatment with PBS, IL-12 (20 μg, i.t.), PD-1 i.p. every 3 days, ATT-02 (i.t, 20 μg), MSR (i.t.) and PD-1 (i.p) every 3 days, ATT-02 (i.t., 20 μg) plus PD-1 (i.p.) every three days. Mice receiving PD-1 was dosed a total of 5 doses PD-1 at 200 μg per mouse. FIG. 24A shows the average tumor volume (mm³) of all treatment groups. FIG. 24B shows the percent change in average weight of treatment groups.

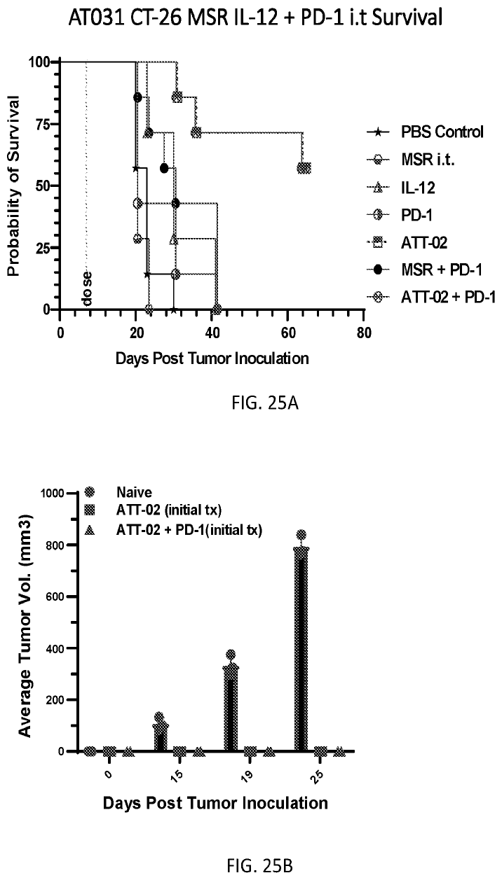

Mice were inoculated with 1M CT-26 cells subcutaneously in right flank on day 0. On day 7 mice began treatment with PBS, IL-12 (20 μg, i.t.), PD-1 i.p. every three days, ATT-02 (i.t, 20 μg), MSR (i.t.) and PD-1 (i.p) every 3 days, ATT-02 (i.t., 20 μg) plus PD-1 (i.p.) every 3 days. Mice receiving PD-1 was dosed a total of 5 doses PD-1 at 200 μg per mouse. FIG. 25A shows percent survival mice by groups with study terminated on day 65. FIG. 25B shows surviving cohort of mice from ATT-02 and ATT-02+PD-1 were re-challenged with 3M CT-26 cells on day 65 and tumor volume measured until day 25 until study was terminated.

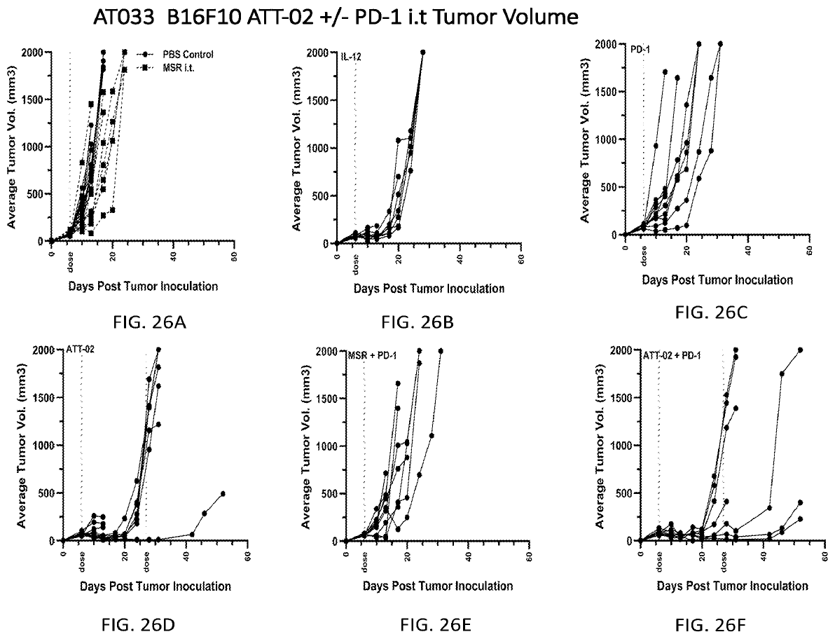

In the next study, mice were inoculated subcutaneously in right flank with 500K B16F10 cells on day 0. On day six, mice began treatment with PBS, IL-12 (20 μg, i.t.), PD-1 i.p. every 3 days, ATT-02 (i.t, 20 μg), MSR (i.t.) and PD-1 (i.p) every three days, ATT-02 (i.t., 20 μg) plus PD-1 (i.p.) every three days. Mice receiving PD-1 was dosed a total of 5 doses PD-1 at 200 μg per mouse. On day 27 A cohort of surviving mice from treatment group ATT-02 and ATT-02+PD-1 received a second dose of ATT-02. FIG. 26A shows individual tumor volumes (mm³) of treatment groups PBS control and MSR i.t. FIG. 26B shows individual tumor volumes (mm³) of IL-12 treatment. FIG. 26C shows Individual tumor volumes (mm³) of treatment with PD-1.

FIG. 26D shows individual tumor volumes (mm³) of treatment group ATT-02. FIG. 26E shows individual tumor volumes (mm³) of treatment group MSR plus anti PD-1. FIG. 26F shows individual tumor volumes (mm³) of treatment group ATT-02 plus anti PD-1.

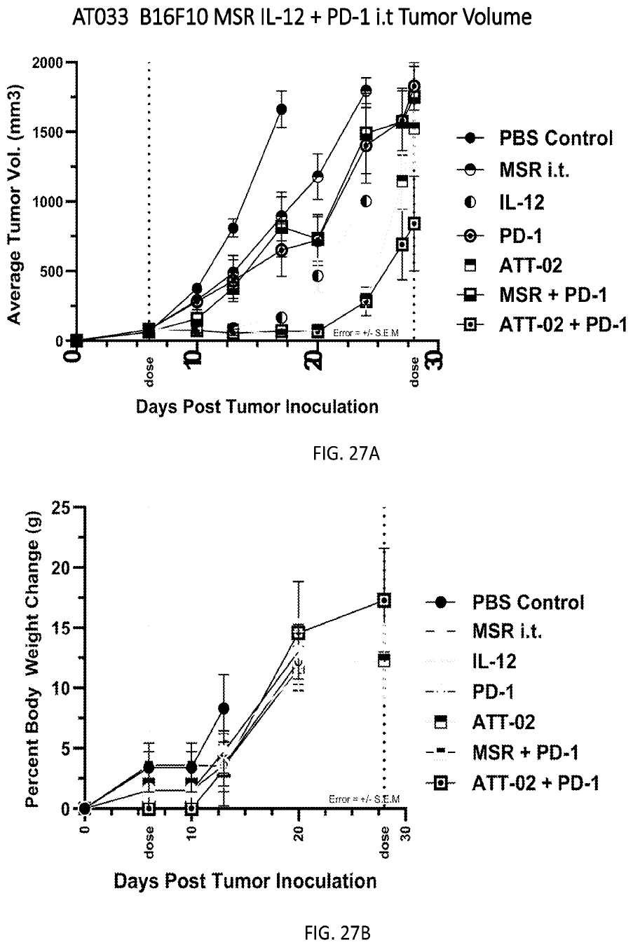

Next, mice were inoculated subcutaneously in right flank with 500K B16F10 cells on day 0. On day six, mice began treatment with PBS, IL-12 (20 μg, i.t.), PD-1 i.p. every 3 days, ATT-02 (i.t, 20 μg), MSR (i.t.) and PD-1 (i.p) every three days, ATT-02 (i.t., 20 μg) plus PD-1 (i.p.) every three days. Mice receiving PD-1 was dosed a total of 5 doses PD-1 at 200 μg per mouse. On day 27 A cohort of surviving mice from treatment group ATT-02 and ATT-02+PD-1 received a second dose of ATT-02. FIG. 27A shows average tumor volume (mm³) of treatment groups and FIG. 27B shows the percent change in average weight of treatment groups.

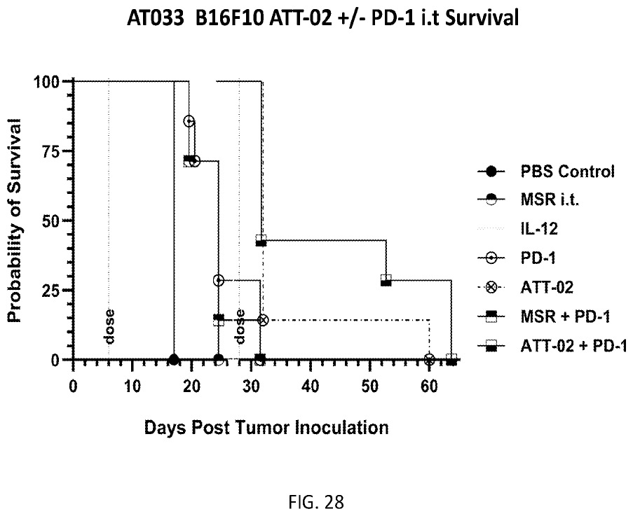

Mice were inoculated subcutaneously in right flank with 500K B16F10 cells on day 0. On day six mice began treatment with PBS, IL-12 (20 μg, i.t.), PD-1 i.p. every three days, ATT-02 (i.t, 20 μg), MSR (i.t.) and PD-1 (i.p) every three days, ATT-02 (i.t., 20 μg) plus PD-1 (i.p.) every three days. Mice receiving PD-1 was dosed a total of 5 doses PD-1 at 200 μg per mouse. On day 27 A cohort of surviving mice from treatment group ATT-02 and ATT-02+PD-1 received a second dose of ATT-02. FIG. 28 shows the percent surviving mice by groups with the study terminated on day 64.

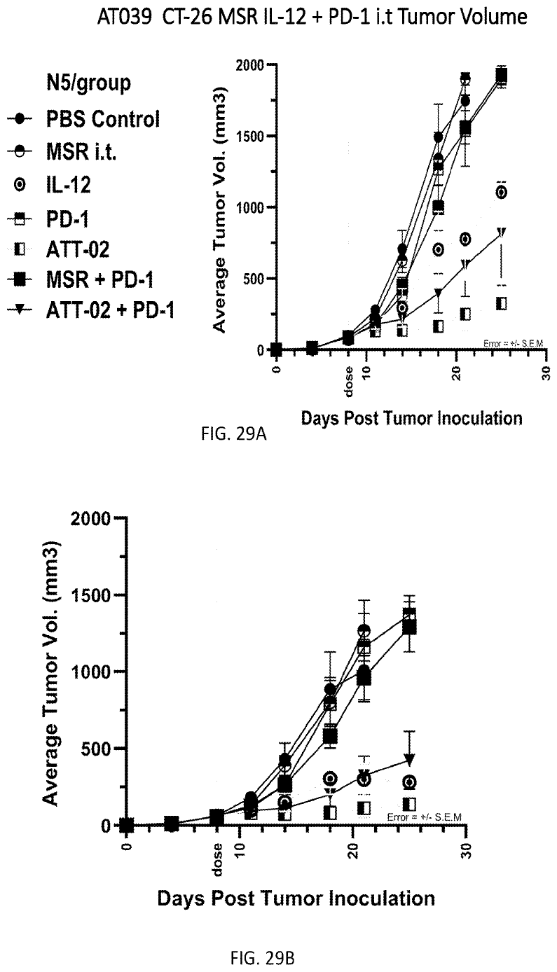

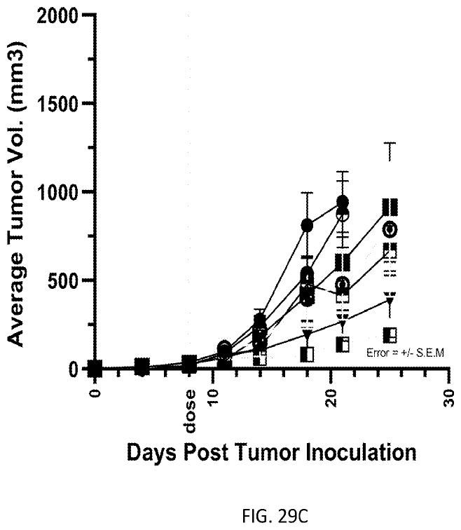

Mice were inoculated subcutaneously with CT-26 cells, 3M cells on right-flank (primary) and 1M cells on left-flank (secondary) on day 0. On day eight, mice were dosed with PBS, MSR (i.t), IL-12 (20 μg, i.t.), PD-1 i.p. every three days, ATT-02 (i.t, 20 μg), MSR (i.t.) plus PD-1 (i.p) every three days, ATT-02 (i.t., 20 μg) plus PD-1 (i.p.) every three days. Mice receiving PD-1 was dosed a total of 5 doses PD-1 at 200 μg per mouse. FIG. 29A shows the average total tumor burden volumes (mm³) of treatment groups. FIG. 29B shows the average tumor volumes (mm³) of the primary right-flank. FIG. 29C shows the average tumor volumes (mm³) of the secondary left-flank.

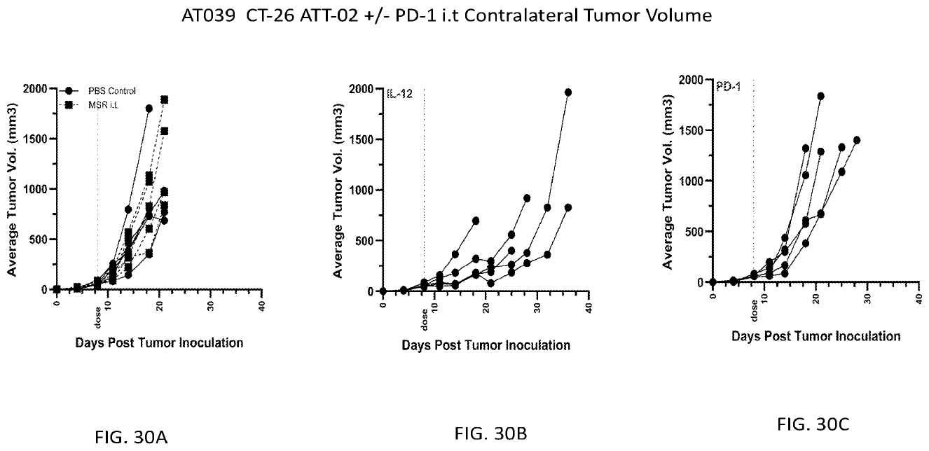

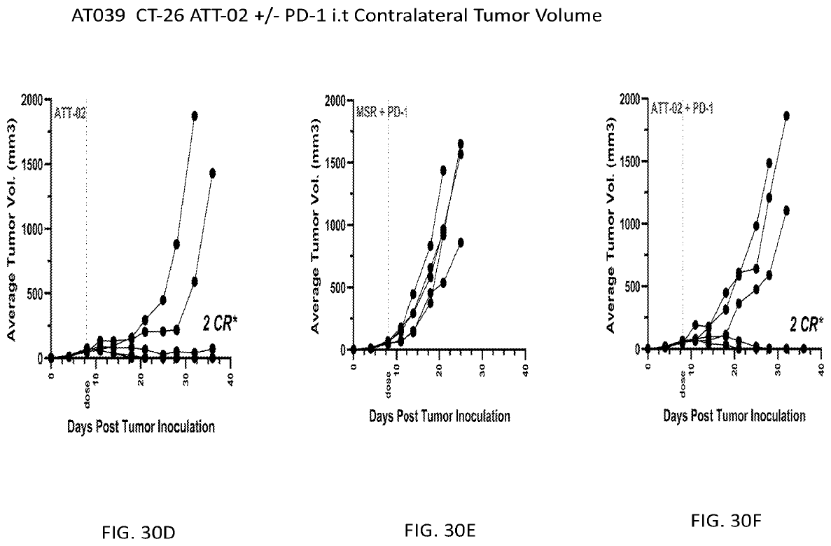

Next, mice were inoculated subcutaneously with CT-26 cells, 3M cells on right-flank (primary) and 1M cells on left-flank (secondary) on day 0. On day eight, mice began treatment with PBS, MSR (i.t), IL-12 (20 μg, i.t.), PD-1 i.p. every three days, ATT-02 (i.t, 20 μg), MSR (i.t.) plus PD-1 (i.p) every three days, ATT-02 (i.t., 20 μg) plus PD-1 (i.p.) every three days. Mice receiving PD-1 was dosed a total of five doses PD-1 at 200 μg per mouse. FIG. 30A shows primary tumor volumes (mm³) of treatment groups PBS control and MSR i.t. FIG. 30B shows the primary tumor volumes (mm³) of IL-12 treatment. FIG. 30C shows the primary tumor volumes (mm³) of PD-1 treatment. FIG. 30D shows the primary tumor volumes (mm³) of treatment group ATT-02. FIG. 30E shows the primary tumor volumes (mm³) of treatment group MSR plus anti PD-1. FIG. 30F shows the primary tumor volumes (mm³) of treatment group ATT-02 plus anti PD-1.

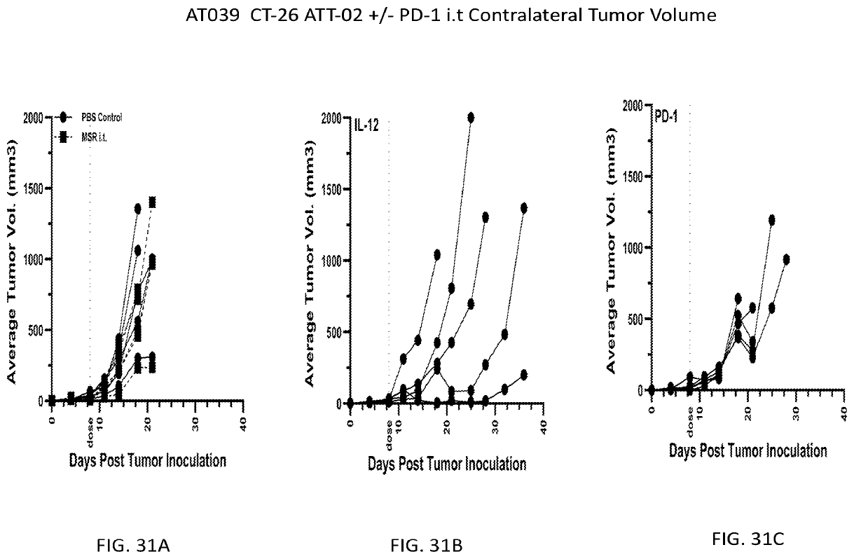

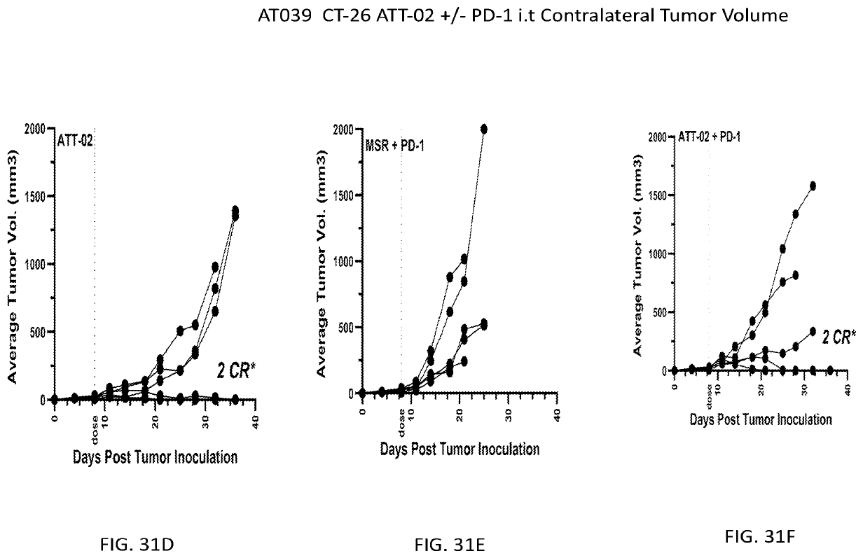

Mice were inoculated subcutaneous with CT-26 cells, 3M cells on right-flank (primary) and 1M cells on left-flank (secondary) on day 0. On day 8 mice began treatment with PBS, MSR (i.t), IL-12 (20 μg, i.t.), PD-1 i.p. every three days, ATT-02 (i.t, 20 μg), MSR (i.t.) plus PD-1 (i.p) every 3 days, ATT-02 (i.t., 20 μg) plus PD-1 (i.p.) every three days. Mice receiving PD-1 was dosed a total of five doses PD-1 at 200 μg per mouse. FIG. 31A shows the secondary tumor volumes (mm³) of treatment groups PBS control and MSR i.t. FIG. 31B shows the secondary tumor volumes (mm³) of IL-12 treatment. FIG. 31C shows the secondary tumor volumes (mm³) of PD-1 treatment. FIG. 31D shows the secondary tumor volumes (mm³) of treatment group ATT-02. FIG. 31E shows the secondary tumor volumes (mm³) of treatment group MSR plus anti PD-1. FIG. 31F shows the secondary tumor volumes (mm³) of treatment group ATT-02 plus anti PD-1.

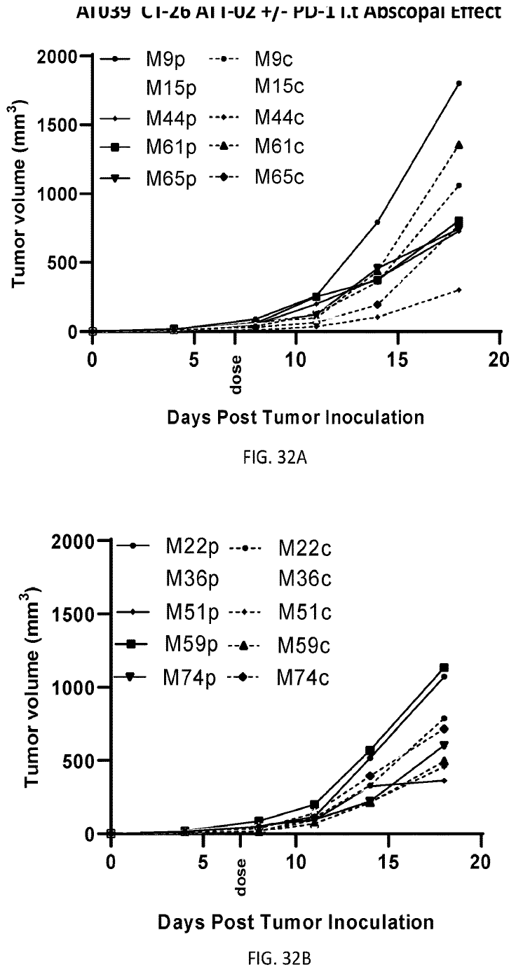

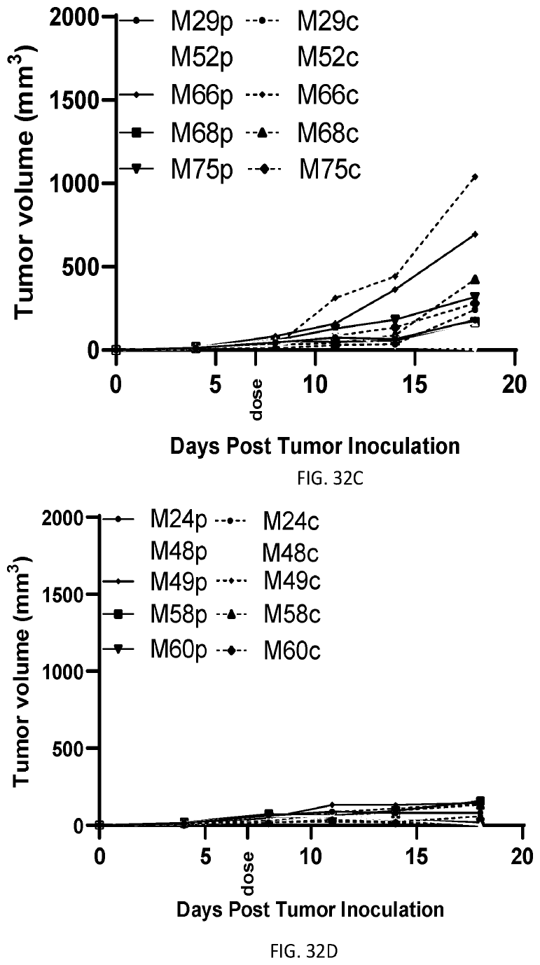

Mice were inoculated subcutaneously with CT-26 cells, 3M cells on right-flank (primary) and 1M cells on left-flank (secondary) on day 0. On day eight, mice were dosed with PBS, MSR (i.t), IL-12 (20 μg, i.t.), PD-1 i.p. every three days, ATT-02 (i.t, 20 μg), MSR (i.t.) plus PD-1 (i.p) every three days, ATT-02 (i.t., 20 μg) plus PD-1 (i.p.) every three days. Mice receiving PD-1 was dosed a total of five doses PD-1 at 200 μg per mouse. FIG. 32A shows the PBS treated mice showing primary versus secondary tumor volumes assessing abscopal effect in two out of five mice. FIG. 32B shows the MSR treated mice showing primary (p) versus secondary (c) tumor volumes assessing abscopal effect in three out of five mice. FIG. 32C shows the IL-12 treated mice showing primary (p) versus secondary (c) tumor volumes assessing abscopal effect in two out of five mice. FIG. 32D shows the ATT-02 treated mice showing primary (p) versus secondary (c) tumor volumes assessing abscopal effect in four out of five mice.

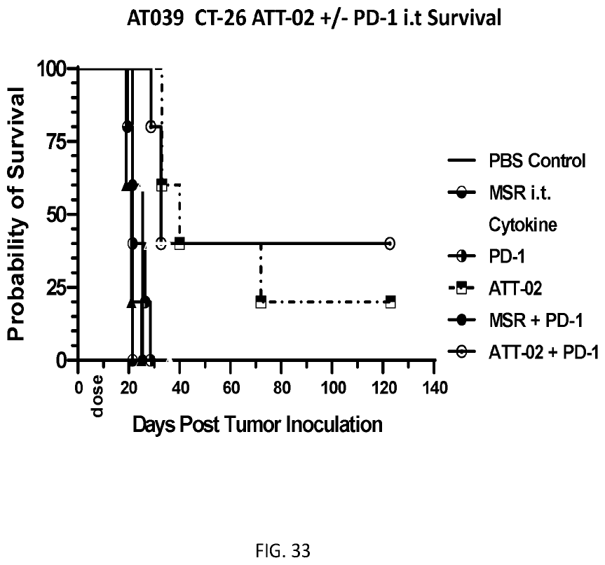

Mice were inoculated subcutaneously with CT-26 cells, 3M cells on right-flank (primary) and 1M cells on left-flank (secondary) on day 0. On day eight, mice were dosed with PBS, MSR (i.t), IL-12 (20 μg, i.t.), PD-1 i.p. every three days, ATT-02 (i.t, 20 μg), MSR (i.t.) plus PD-1 (i.p) every three days, ATT-02 (i.t., 20 μg) plus PD-1 (i.p.) every three days. Mice receiving PD-1 was dosed a total of five doses PD-1 at 200 μg per mouse. FIG. 33 shows the percent surviving mice by treatment groups. The study was terminated on day 122.

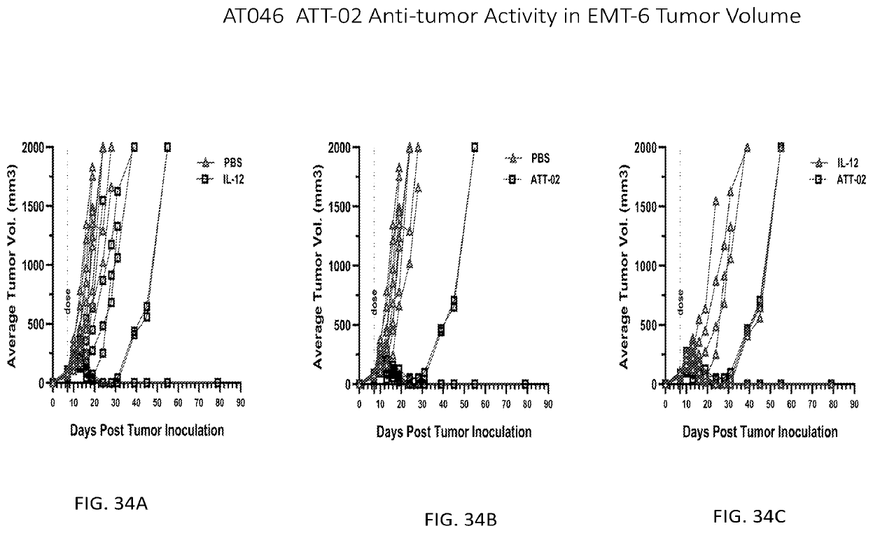

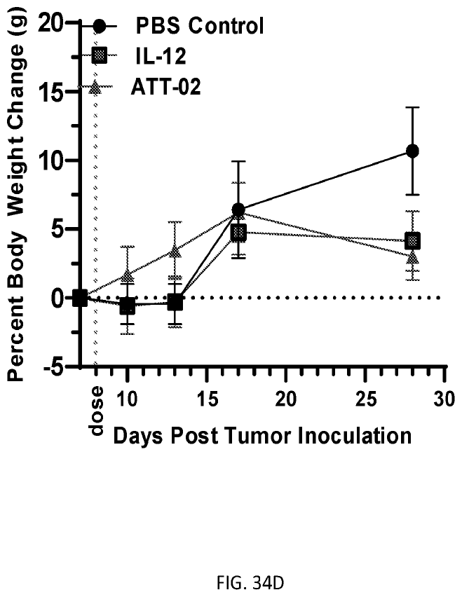

Mice were inoculated with 1M EMT-6 cells subcutaneously in right flank on day 0. On day seven mice were dosed with PBS, IL-12 (20 pg, i.t.), or ATT-02 (i.t, 20 μg), FIG. 34A shows the tumor volumes (mm³) of treatment groups PBS control compared to IL-12 i.t. FIG. 34B shows the tumor volumes (mm³) of PBS control compared to ATT-02 treatment. FIG. 34C shows the tumor volumes (mm³) of treatment ATT-02 compared to IL-12. FIG. 34D shows the percent change in mice body weight over the course of the study.

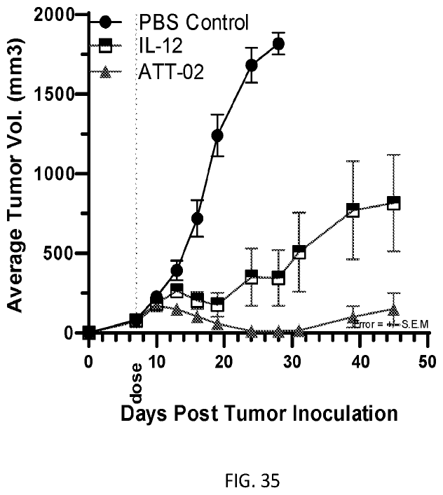

Mice were inoculated with 1M EMT-6 cells subcutaneously in right flank on day 0. On day seven mice were dosed with PBS, IL-12 (20 μg, i.t.), or ATT-02 (i.t, 20 μg). FIG. 35 shows average tumor volumes (mm³) of the treatment groups.

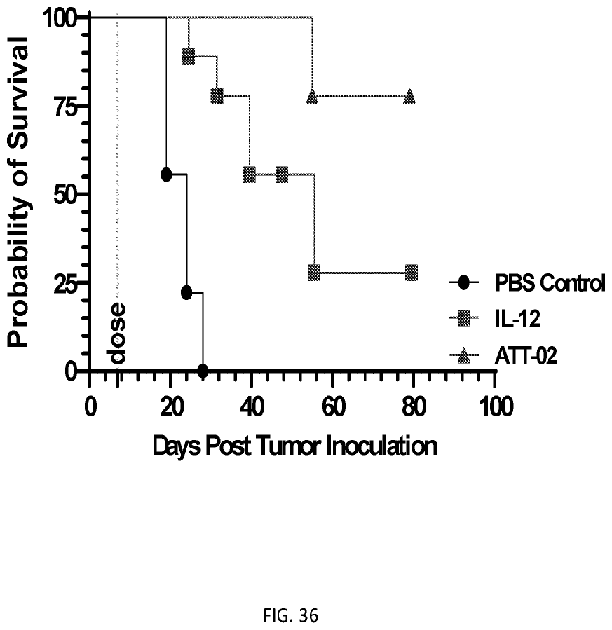

Next, mice were inoculated with 1M EMT-6 cells subcutaneous in right flank on day 0. On day 7 mice were dosed with PBS, IL-12 (20 μg, i.t.), or ATT-02 (i.t, 20 μg). FIG. 36 shows a survival analysis by treatment, with the study terminating on day 80.

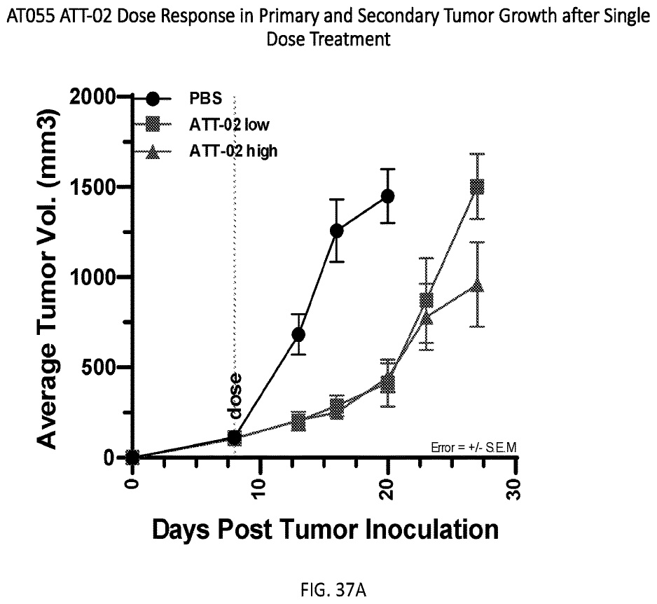

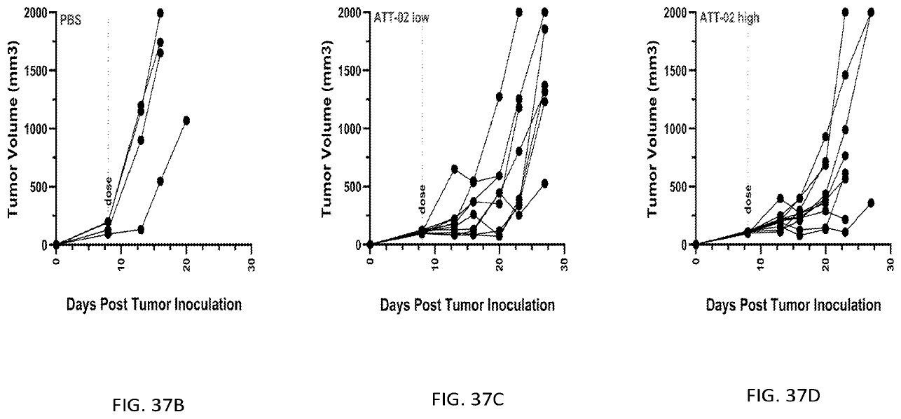

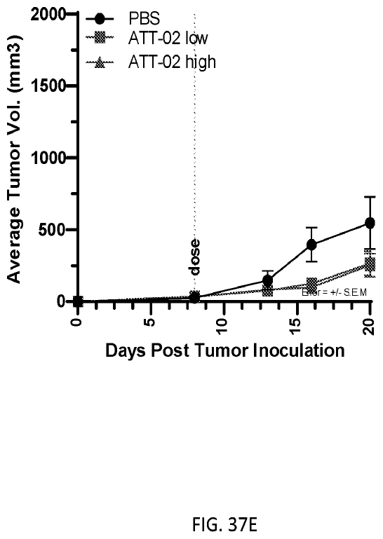

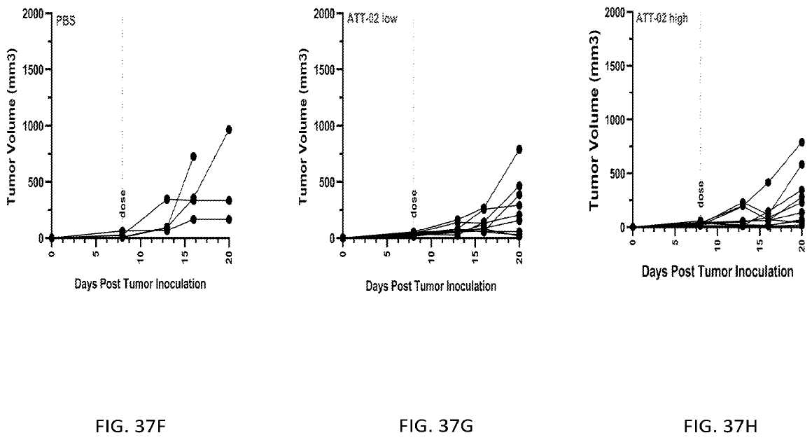

Mice were inoculated subcutaneously with B16F10 cells, 1M cells on right-flank (primary) and 0.25M cells on left-flank (secondary) on day 0. On day eight, mice were dosed with PBS or ATT-02 at two dose levels, low dose (28 μg) or high (56 μg) i.t. FIG. 37A shows the average primary tumor burden volumes (mm³) of treatment groups in the single dose mice. FIG. 37B shows the tumor volumes (mm³) of the primary right-flank of the PBS control. FIG. 37C shows the tumor volumes (mm³) of the primary right-flank of the ATT-02 low treatment. FIG. 37D shows the tumor volumes (mm³) of the primary right-flank of the ATT-02 high treatment. FIG. 37E shows the average secondary tumor burden volumes (mm³) of treatment groups in the single dose mice. FIG. 37F shows the tumor volumes (mm³) of the secondary left-flank of the PBS control. FIG. 37G shows the tumor volumes (mm³) of the secondary left-flank of the ATT-02 low treatment. FIG. 37H shows the tumor volumes (mm³) of the secondary left-flank of the ATT-02 high treatment.

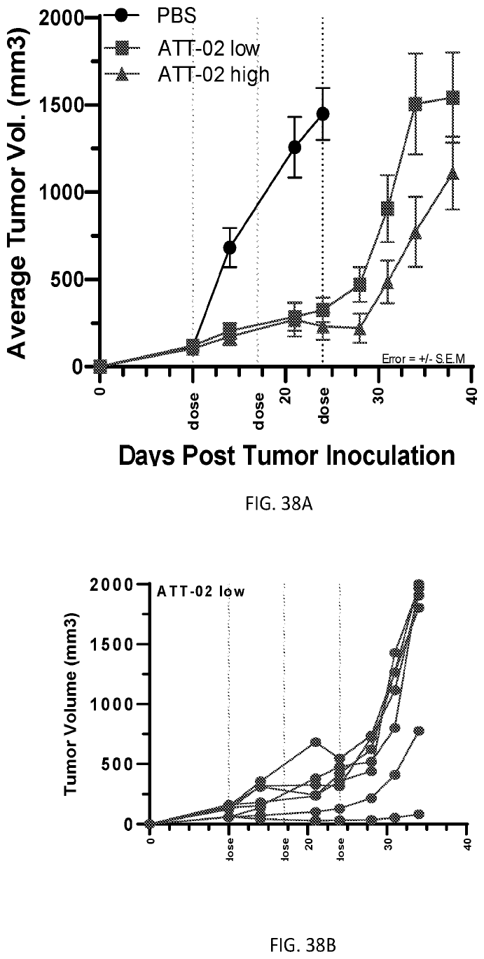

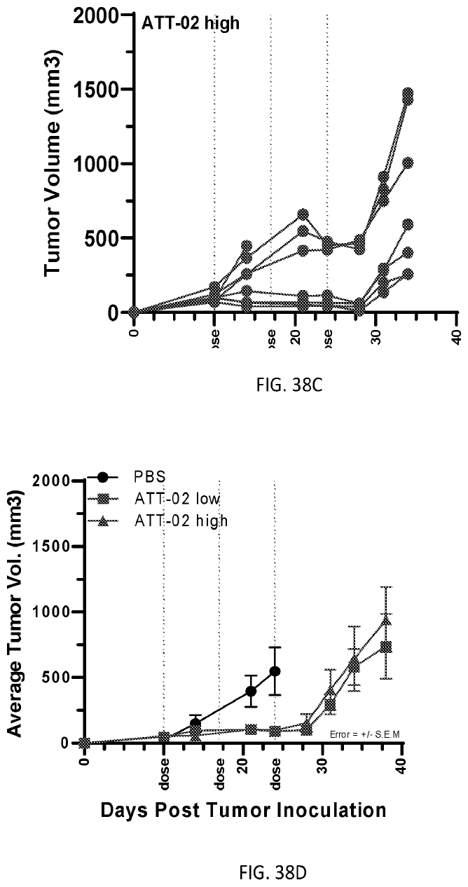

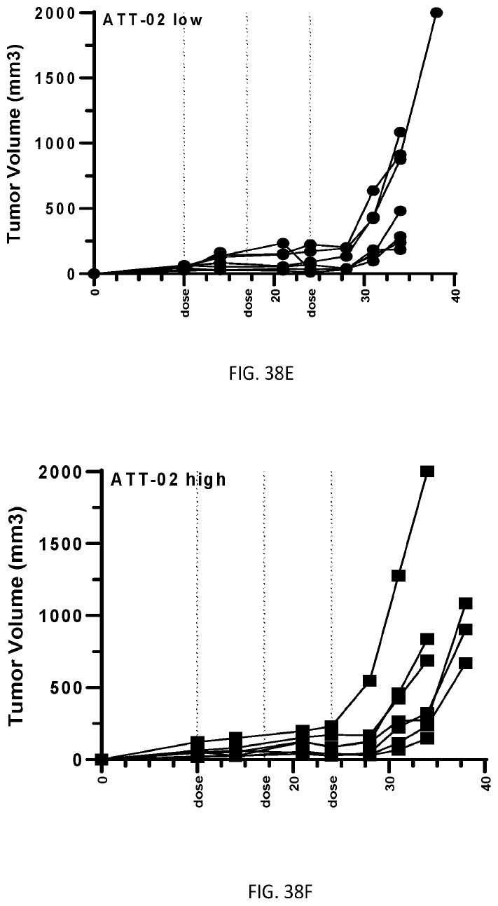

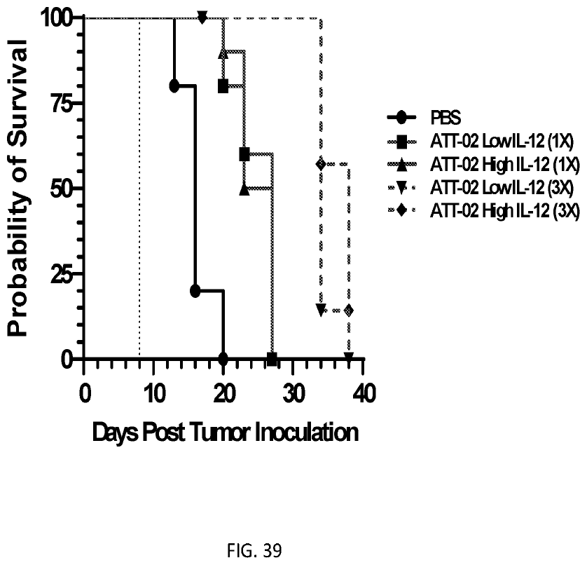

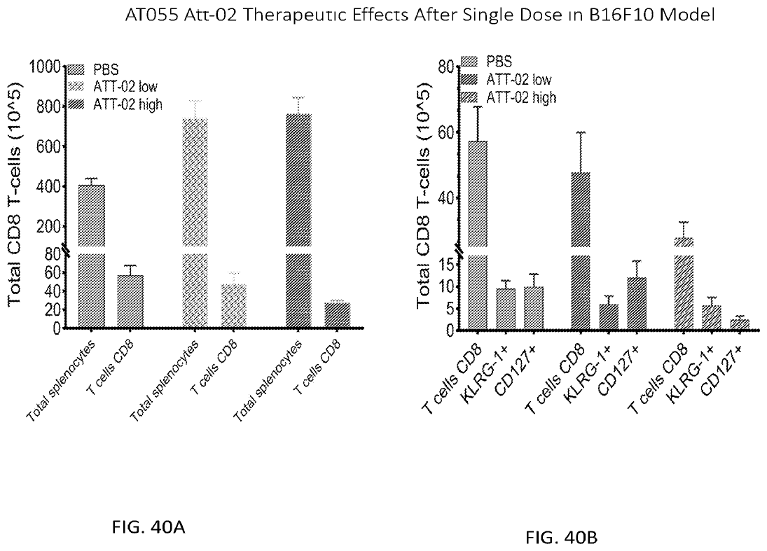

Next, mice were inoculated subcutaneously with B16F10 cells, 1M cells on right-flank (primary) and 0.25M cells on left-flank (secondary) on day 0. On day 10, 17 and 24 mice were dosed with PBS or ATT-02 at two dose levels, low dose (28 μg) or high (56 μg) i.t. FIG. 38A shows the average primary tumor burden volumes (mm³) of treatment groups in the multi-dose mice. FIG. 38B shows the tumor volumes (mm³) of the primary right-flank of the PBS control multi-dose. FIG. 38C shows the tumor volumes (mm³) of the primary right-flank of the ATT-02 low treatment multi-dose. FIG. 38D shows the average secondary tumor burden volumes (mm³) of multi-dose treatment groups. FIG. 38E shows the tumor volumes (mm³) of the secondary left-flank of the ATT-02 low multi-dose treatment. FIG. 38F shows the tumor volumes (mm³) of the secondary left-flank of the ATT-02 high multi-dose treatment. FIG. 39 shows probability of survival.

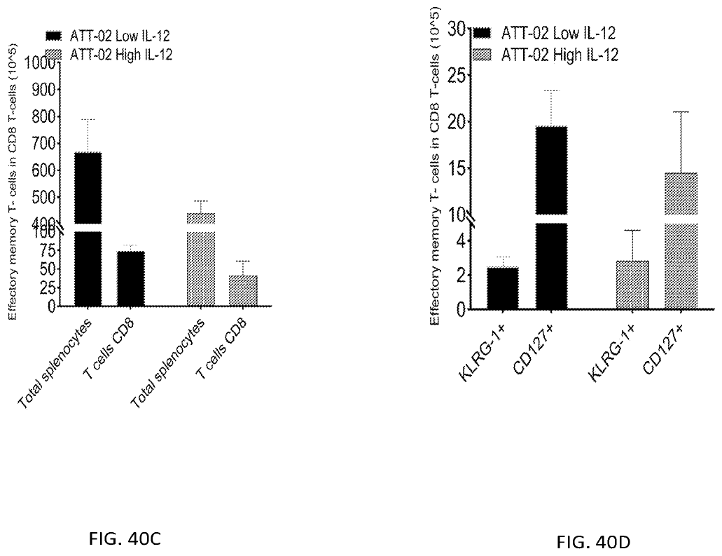

Mice were inoculated subcutaneous with B16F10 cells, 1M cells on right-flank (primary) and 0.25M cells on left-flank (secondary) on day 0. Single dose cohort (1×) received a dose on day eight with PBS or ATT-02 at two dose levels, low dose (28 μg) or high (56 μg) i.t. Splenocyte collected on day 7 and 14 post a single dose. FIGS. 40A and 40B show day seven total CD8+ T-cells, KLRG1 and CD127+ cells. FIGS. 40C and 40D show day 14 total CD8+ T-cells, KLRG1 and CD127+ cells. In summary:
1M splenocytes collected for Flow
Gating on live population
SCC-A vs CD8+ for T-cells.
KLRG-1 and CD127 in CD8+ cells calculated back to the total cell number

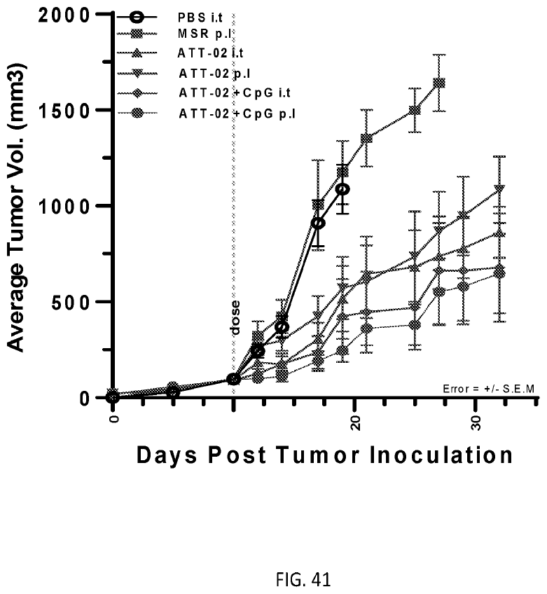

Mice were inoculated subcutaneously with B16F10 cells, 1M cells on right-flank (primary) and 0.25M cells on left-flank (secondary) on day 0. On day ten, mice were dosed with PBS or ATT-02+/−CpG either perilymphatic (p.l) or intratumoral (i.t). FIG. 41 shows the average primary tumor burden volumes (mm³) of the treatment groups.

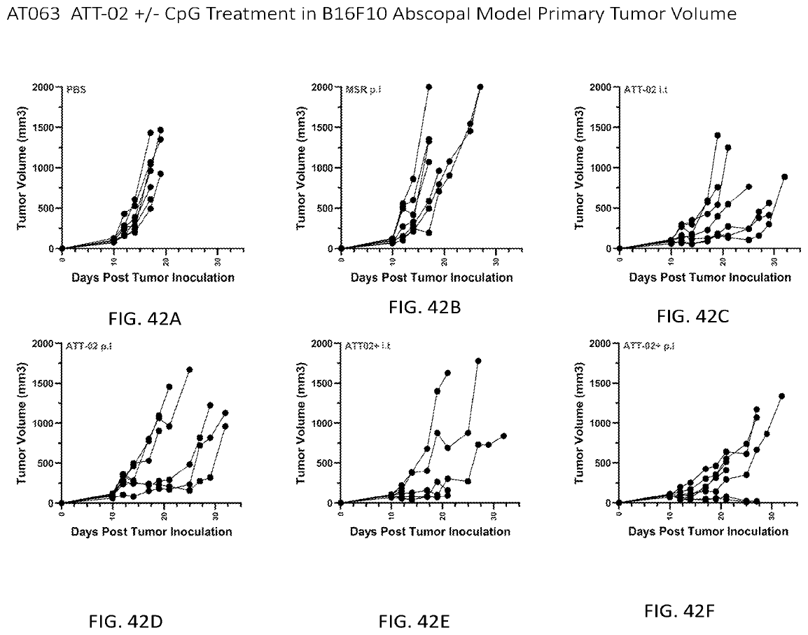

Next, mice were inoculated subcutaneously with B16F10 cells, 1M cells on right-flank (primary) and 0.25M cells on left-flank (secondary) on day 0. On day 10, mice were dosed with PBS or ATT-02+/−CpG either perilymphatic (p.l) or intratumoral (i.t). FIG. 42A shows the tumor volumes (mm³) of treatment group PBS control. FIG. 42B shows the tumor volumes (mm³) of MSR perilymphatic treatment. FIG. 42C shows the tumor volumes (mm³) of ATT-02 i.t treatment. FIG. 42D shows the tumor volumes (mm³) of treatment group ATT-02 perilymphatic. FIG. 42E shows the tumor volumes (mm³) of treatment group ATT-02 plus CpG i.t. FIG. 42F shows the tumor volumes (mm³) of treatment group ATT-02 plus CpG perilymphatic.

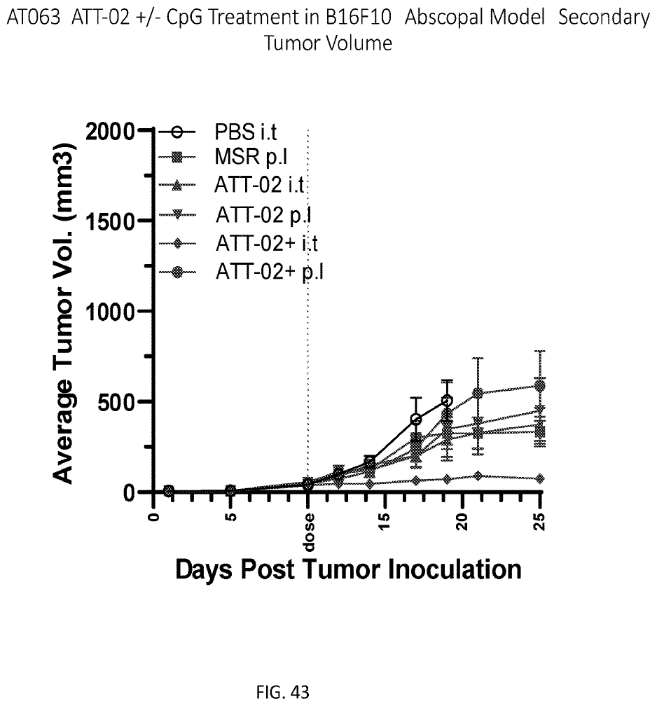

Mice were inoculated subcutaneously with B16F10 cells, 1M cells on right-flank (primary) and 0.25M cells on left-flank (secondary) on day 0. On day 10, mice were dosed with PBS or ATT-02+/−CpG either perilymphatic (p.l) or intratumoral (i.t). FIG. 43 shows the average secondary tumor burden volumes (mm³) of the treatment groups.

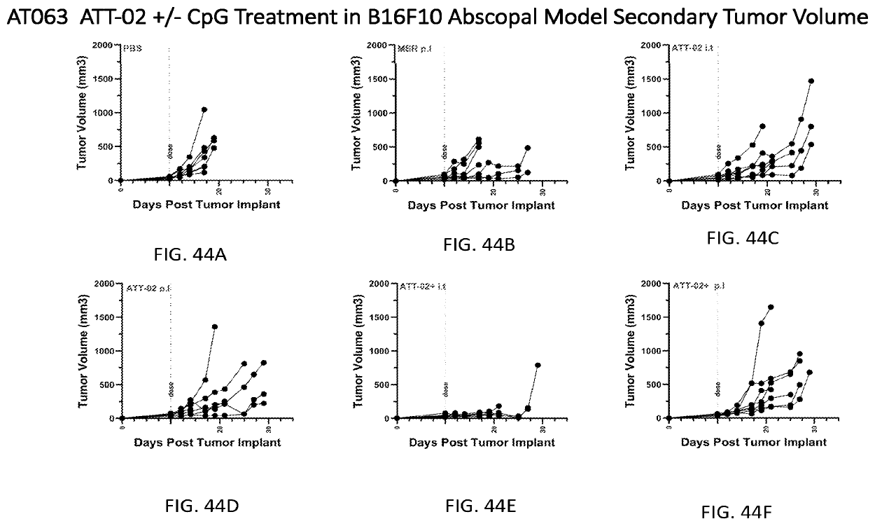

Mice were inoculated subcutaneously with B16F10 cells, 1M cells on right-flank (primary) and 0.25M cells on left-flank (secondary) on day 0. On day 10, mice were dosed with PBS or ATT-02+/−CpG either perilymphatic (p.l) or intratumoral (i.t). FIG. 44A shows the secondary tumor volumes (mm³) of treatment group PBS control. FIG. 44B shows the secondary tumor volumes (mm³) of MSR perilymphatic treatment. FIG. 44C shows the secondary tumor volumes (mm³) of ATT-02 i.t treatment. FIG. 44D shows the secondary tumor volumes (mm³) of treatment group ATT-02 perilymphatic. FIG. 44E shows the secondary tumor volumes (mm³) of treatment group ATT-02 plus CpG i.t. FIG. 44F shows the secondary tumor volumes (mm³) of treatment group ATT-02 plus CpG perilymphatic.

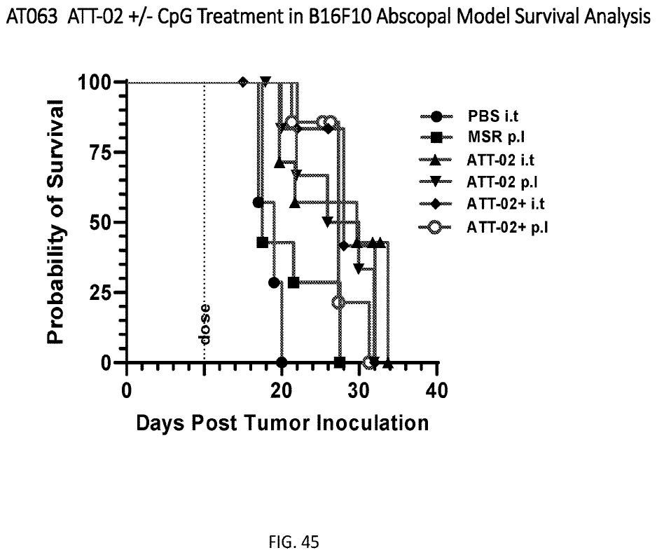

Mice were inoculated subcutaneously with B16F10 cells, 1M cells on right-flank (primary) and 0.25M cells on left-flank (secondary) on day 0. FIG. 45 shows the survival analysis between the treatment groups. The study was terminated on day 34.

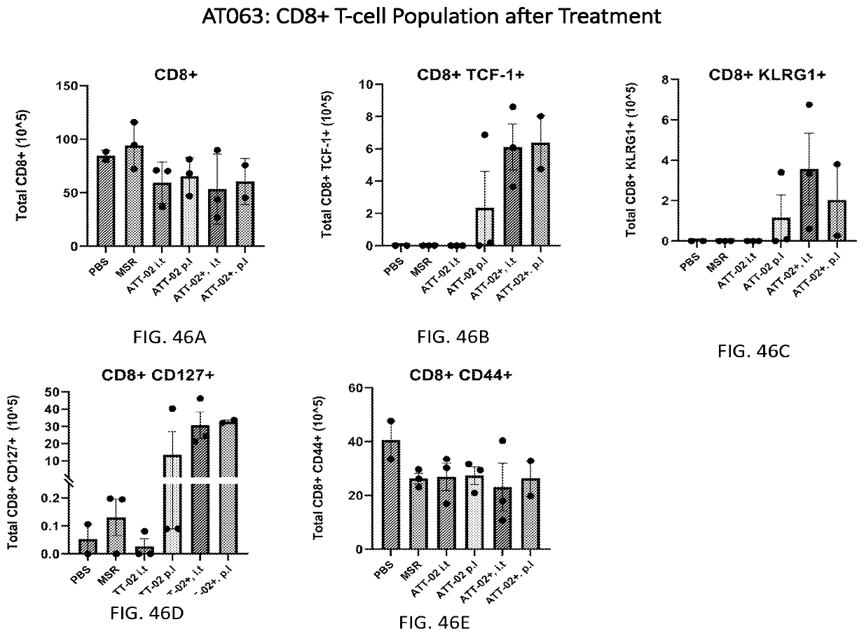

Mice were inoculated subcutaneously with B16F10 cells, 1M cells on right-flank (primary) and 0.25M cells on left-flank (secondary) on day 0. On day 10, mice were dosed with PBS or ATT-02+/−CpG either perilymphatic (p.l) or intratumoral (i.t). Splenocyte collected on day 7 post dose for FLOW analysis. 1M splenocytes collected for Flow with gating on live population SCC-A vs CD8+ for T-cells TCF-1+, KLRG1+, CD44+ and CD127 in CD8+ population for regulatory and activated T-cells calculated back to the total cell number. FIG. 46A shows total CD8+ T cells population. FIG. 46B shows CD8+ TCF-1+ population. FIG. 46C shows CD8+ KLRG1+ population. FIG. 46D shows the CD8+CD127+ population. And FIG. 46E shows the CD8+CD44+ population.

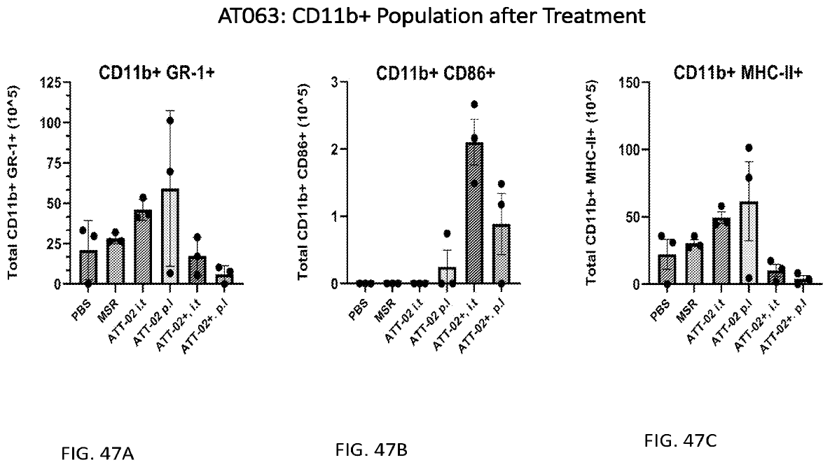

Mice were inoculated subcutaneously with B16F10 cells, 1M cells on right-flank (primary) and 0.25M cells on left-flank (secondary) on day 0. On day 10, mice were dosed with PBS or ATT-02+/−CpG either perilymphatic (p.l) or intratumoral (i.t). Splenocyte collected on day 7 post dose for FLOW analysis. 1M splenocytes collected for Flow with gating on live population CD11b+GR-1+; CD11b+CD86+; CD11b+MHC-II+ for macrophages and monocytes calculated back to the total cell number. FIG. 47A shows total CD11b+GR-1+ population. FIG. 47B shows CD11b+CD86+ population. FIG. 47C shows CD11b+MHCII+ population.

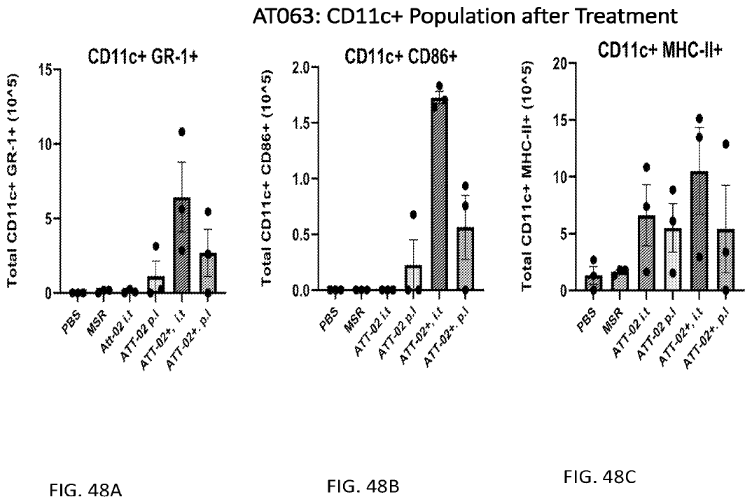

As above, mice were inoculated subcutaneous with B16F10 cells, 1M cells on right-flank (primary) and 0.25M cells on left-flank (secondary) on day 0. On day 10, mice were dosed with PBS or ATT-02+/−CpG either perilymphatic (p.l) or intratumoral (i.t). Splenocyte collected on day seven post dose for FLOW analysis. 1M splenocytes collected for Flow with gating on live population CD11c+GR-1+; CD11c+CD86+; CD11c+MHC-II+ for macrophages and monocytes calculated back to the total cell number. FIG. 48A shows total CD11c+GR-1+ population. FIG. 48B shows CD11c+CD86+ population. FIG. 48C shows CD11c+MHCII+ population.

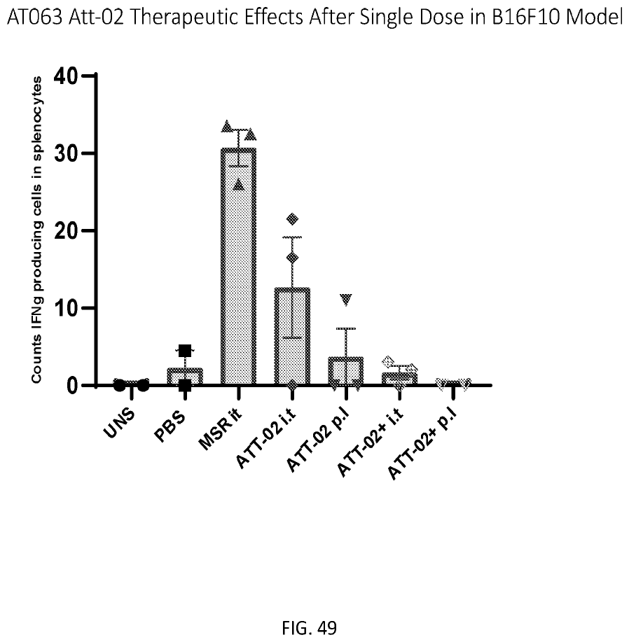

Mice were inoculated subcutaneously with B16F10 cells, 1M cells on right-flank (primary) and 0.25M cells on left-flank (secondary) on day 0. On day ten, mice were dosed with PBS or ATT-02+/−CpG either perilymphatic (p.l) or intratumoral (i.t). Splenocyte collected on day seven post dose for interferon gamma (IFNg) analysis. 1M splenocytes were co-cultured with either B16 peptides: 30 μg each (B16-M27, B16-M40, B16-M27, B16-M47, B16-M48) or B16F10 cell lysate. Unstimulated cells used for controls. FIG. 49 shows the IFN gamma expression by treatment groups.

### Example 11

Additional Studies in Mice Treated with MSRs

Despite the remarkable clinical success of immune checkpoint inhibitor (ICI) therapy in advanced cutaneous melanoma (skin cutaneous melanoma; SKCM), a high unmet medical need persists as only a subpopulation of these patients respond and many of these patients subsequently progress. In advanced SKCM patients treated with pembrolizumab monotherapy, for example, approximately 60% fail to respond and median progression free survival (PFS) is about 5.6 months. Myeloid cell populations enforce the immunosuppressive program characteristic of the tumor microenvironment. Indeed, the presence of immunosuppressive tumor associated macrophages (TAMs) and other anti-inflammatory, myeloid-lineage cells strongly correlate with poor prognosis across multiple tumor types and therapies, including resistance to anti-PD(L)-1 therapeutics. Although an oversimplification, the M1/M2 paradigm captures the functional antipodes of macrophage states, where M1 macrophages are strongly inflammatory/immunogenic and M2 macrophages are strongly immunosuppressive. TAMs are known to be phenotypically plastic—occupying metastable functional states rather than locked-in phenotypes—and remain responsive to local signals which influence their functional states. This opens up the possibility of therapeutic conversion of M2 immunosuppressive TAMs into an immunogenic M1 population.

Interleukin-12 (IL-12) is a potent pro-inflammatory cytokine, mainly expressed by activated innate immune cells, including M1 macrophages, neutrophils and dendritic cells, which provides a pivotal linkage between upstream “danger sensing” and downstream adaptive immune responses [16-19]. Unfortunately, systemic delivery of IL-12 efforts has been hampered by the toxicity associated with systemic exposure.

However, as the critical locus of action is within the tumor microenvironment (TME), intralesional IL-12 therapy has been actively pursued as an intratumoral therapy. Importantly, IL-12 activates NK and antigen-experienced T cells, leading to secretion of IFNγ, which, in turn, further upregulates IL-12 expression, creating a pivotal feed-forward activation loop. When established within the TME, this “IL-12/IFNγ cycle” is capable of converting immunosuppressive TAMs into immunogenic “M1” macrophages. In a landmark paper in 2011, Restifo and colleagues demonstrated that IL-12 upregulates critical components of antigen processing and presentation machinery in TAMs enabling them to effectively cross-present tumor antigens from dying tumor cells. Furthermore, these converted inflammatory TAMs express critical costimulatory molecules, pro-inflammatory cytokines, chemokines and other factors, required for driving an effective type I anti-tumor immune response. Thus, intralesional IL-12 is sufficient to drive an effective “in situ vaccination.” A clear advantage to an in situ vaccine approach over an antigen-specific vaccine is that dead tumor cells represent the antigen source, allowing the patient's own immune system to “personalize” the selection of optimal immunogenic tumor antigens.

One of the leading IL-12-based in situ vaccine approaches has been intralesional IL-12 gene delivery employing DNA-encoded IL-12 (tavokinogene telseplasmid; tavo) and in vivo electroporation (Tavo-EP). Although clear immunologic activity and sporadic clinical benefit was observed, both in preclinical models and in patients with melanoma, Merkel cell carcinoma and TNBC, this approach failed to meet its pre-specified ORR in combination with pembrolizumab in a recent Phase 2 trial in advanced cutaneous melanoma patients, who were refractory or who progressed on prior anti-PD(L)-1 therapy. Applicants hypothesize that the clinical failure of Tavo-EP was the consequence of a hard-to-control drug delivery system compounded by the intrinsic heterogeneity of the tumor microenvironment, which likely varied in terms of the density of both IL-12-responsive/IFNγ-secreting cells (NK and T cells) and IFNγ-responsive TAMs.

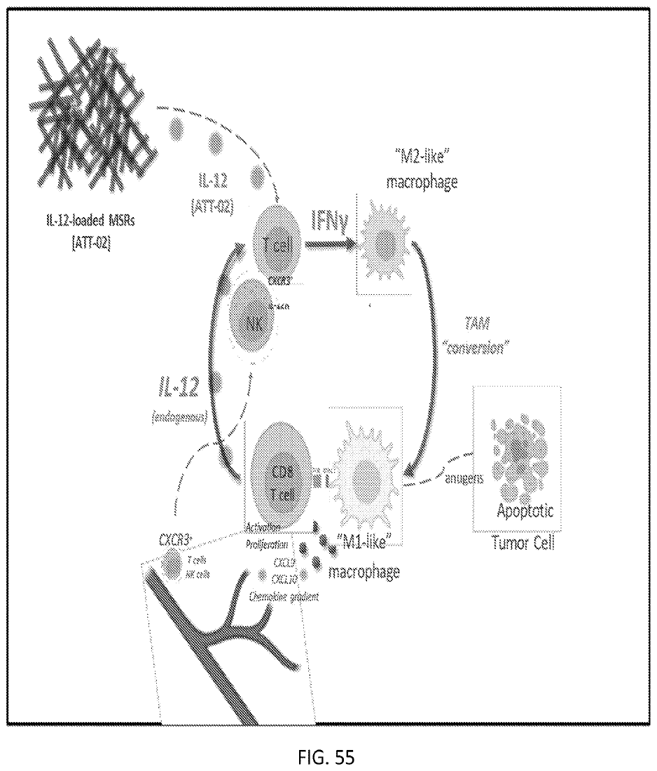

Importantly, analysis of both clinical trial samples and experimental models has underscored the criticality of activating the IFNγ/CXCL9/CXCR3 cytokine/chemokine cascade to enable both successful in situ vaccination as well as responses to anti-PD(L)-1 agents. Indeed, analysis of melanoma tumors from a Phase 2 clinical trial of Tavo-EP plus pembrolizumab in low-TIL patients, who typically fail to respond to pembrolizumab monotherapy, demonstrated that induction of intratumoral CXCR3 mRNA within the TME correlated strongly with clinical response, an effect presumed to be due to increased infiltration of CXCR3+ T cells and NK cells into the tumor. Using both experimental mouse TNBC models and patient samples from a Phase 2 “window-of-opportunity” trial, Telli et al. also identified that response to intralesional Tavo-EP required activation of the IL-12/IFNγ cycle with downstream induction of the IFNγ/CXCL9/CXCR3 recruitment pathway. Taken together, these multiple preclinical and clinical studies indicate that activation of the IL-12/IFNγ loop can successfully convert the TME/tumor immunity cycle from a tolerogenic state into an inflammatory, immunogenic one. Importantly, however, the feed-forward amplification required to achieve this TME “tipping point” depends on the presence of IL-12-responsive/IFNγ-secreting cells (e.g. T cells, NK cells) AND IFNγ-responsive cells (e.g. TAMs, DCs), which secrete IL-12 and IFNγ-dependent chemokines such as CXCL9 and CXCL10. Secretion of these T cell and NK cell chemokines can further amplify the cycle by recruiting IL-12 responsive CXCR3+ T cells and NK cells to the TME. The diagram (FIG. 55) outlines these key facets of the IL-12/INFγ cycle. The goal of the proposed grant is to test a set of known immunomodulatory molecules for the ability to enhance IL-12's ability to trigger the IL-12/INF gamma cycle within the TME and to drive durable anti-tumor T cell activity and tumor regression.

FIG. 55 depicts the IL-12 In Situ Vaccine Paradigm. The IL-12 in situ vaccine effect depends upon activation of the IL-12/IFNγ feed-forward loop in the tumor microenvironment (TME). IL-12 activates intratumoral antigen-experienced T cells and NK cells, leading to secretion of IFNγ. IFNγ then activates tumor associated macrophages (TAMs). leading to conversion of immunosuppressive “M2-like” TAMs into inflammatory, cross-presenting “M1-like” macrophages. IFNγ-stimulated TAMs, in turn, secrete chemokines (e.g. CXCL9 and CXCL10), which recruit new CXCR3+ antigen-experienced T cells and NK cells into the TME. Upon exposure to IL-12, these newly recruited inflammatory cells acquire enhanced cytotoxic activity and secrete IFNγ, further amplifying the cycle.

By (1) utilizing a high-payload capacity biomaterial that insures sustained and controlled drug exposure(s) within the TME and (2) identifying complementary immune activating molecules that recruit IL12 responsive cells into the TME. The MSR-based drug delivery system has several novel features, which address the limitations of previous IL-12-based in situ vaccines. MSRs are silica rods approximately 100 microns in length and 5 microns in diameter containing abundant smaller pores, averaging 5-10 nm in diameter, resulting in a tremendous surface area (~900 m²/mg MSR), which facilitates adsorption of immunomodulatory proteins (e.g. IL-12). In addition, MSRs display intrinsic pro-inflammatory effects, which has been demonstrated to act through NLR3-dependent inflammasome activation. In the context of an in-situ vaccine, the proinflammatory effects of mesoporous silica likely adds a significant adjuvant effect. Thus, ATT-02 already represents a combination immunomodulatory modality. Another critical innovation, intrinsic to the platform, is its modularity. Individual batches of MSRs can be loaded with a single immune stimulating protein and then a combination product can then be easily assembled simply by mixing pre-loaded MSRs. This modularity eases the formulation and testing of candidate IL-12 combinations where with other systems, it would not be technically feasible to test multiple combinations, including doublets, triplets, etc. Lastly, MSRs—unlike many biomaterial drug delivery solutions—can be easily manufactured at scales appropriate for clinical trial usage and commercialization.

In vitro testing of ATT-02 (IL-12 monotherapy) versus ATT-02+ (IL-12 in combination with additional well-characterized immune activating agents) to optimize “conversion” of immunosuppressive immature myeloid APC into activated, immunogenic APC with enhanced ability for cross-presentation (referred to as “Aim 1).

As the goal of Aim 1 is to screen for potential immune activators that synergize with IL-12, Applicants developed an in vitro system to enable efficient screening by focusing on the ability to drive effective cross-presentation by macrophages using ovalbumin-specific CD8 T cells from the OT-1 transgenic mouse as a read-out. In brief, transwell inserts with an 8-micron pore size will be coated with matrigel to facilitate injection of MSRs. M2 macrophages will be generated from bone marrow harvested from C57BL/6 H2-Kb+ mice per standard protocol. After polarization, the M2 macrophages will be harvested, counted and 0.5×106 cells will be plated onto the matrigel. 1×106 OT-1 CD8 T cells isolated from OT-1 transgenic mouse spleens will be labelled with the fluorescent dye, CFSE, in order to track proliferation by flow. A total of 1 mg of MSRs will be injected into the matrigel layer of each insert with the total amount of MSR held constant. The MSRs will contain no payload, IL-12 alone or various combinations of the immune activating molecules outlined below.

(Table 3). In previous studies, Applicants established that successful IL-12 mediated in situ vaccination is characterized by the generation of a unique KLRG1hiPD-1loCTLA-4lo phenotype within the CD8 population which is similar to what has been identified in viral immunology as short-lived effector cells (aka SLECs). After 72 hours of incubation, cells will be harvested and analyzed by flow cytometry to assess the extent of OT-1 proliferation and the percentage of CD8 T cells that exhibit this unique “SLEC-like” effector T cell phenotype. The flow panel will include the following: live/dead discriminator dye, CSFE, CD8, KLRG1, CD127, PD-1, CTLA-4, CD107a, CD44, CD69. Positive controls will include stimulation of proliferation with CD3/28 activation beads.

Anticipated Results/Pitfalls/Considerations: Although the present technology can accommodate a combination approach with a plethora of components, ATT-02+ may preferably be limited to a total number of three payloads in order to limit the cost and complexity of product manufacturing. The proposed molecules to be combined with IL-12 have published data supporting the enhancement either of M1 macrophage polarization, cross-presentation or in-situ vaccine effects (references included in Table 3). Applicants anticipate the identification of several IL-12+“X” doublets and IL-12+“X”+“Y” triplets, which result in a significant increases in (1) OT-1 proliferation and (2) percentage of CD8 cells with a KLRG1+ phenotype. Because the endpoints are both quantitative with continuous variable read-out, we anticipate no problems with ranking the combinations. Each of 45 conditions (no treatment/MSR alone/IL-12 alone/IL-12+ “X” (n=6)/IL-12+“X”+“Y” (n=36) will be repeated in quadruplicate to assess variance and achieve confidence in the coherence of signal.

Subsequently, a second experiment was performed whereby “IL-12 only” is compared against the top 5-ranked combinations and compared using a 1-WAY ANOVA with Bonferroni correction for multiple pairwise comparisons. If ranking by OT-1 proliferation and percentage of KLRG1+OT-1 cells fail to discriminate between these combinations, then Applicants will consider increasing the stringency of the assay by enhancing the immunosuppressive phenotype of the macrophages with hydrocortisone and IL-10.
Milestone Aim 1: The best-performing IL-12 combination is compared against IL-12 alone (aka ATT-02) in vivo in Aim 2. If no combination increases OT-1 proliferation or frequency of KLRG1+OT-1 cells by >50%, then only ATT-02 is studied further in Aim 2 and Aim 3.

TABLE 3
Immune activating molecules screened for synergy with ATT-02

| | Immune | | |
|---|---|---|---|
| | Activating | | |
| | Molecule | Reagent | Rationale |
| 1 | TLR9 agonist | ODN1585 VacciGrade ™ | TLR9 agonist; combinatorial activity with IL- |
| | (CpG) | (InvivoGen ™) | 12 (Sagiv-Barfi_S, 2022) |
| 2 | TLR3 agonist | Poly (I:C) HMW VcclGrade ™ | TLR3 agonist; activity in intralesional E7 peptide |
| | (Poly I:C) | (InvivoGen ™) | vaccine (Ishida, |
| 3 | CD40 agonist | Clone FGK45 mAb (Thermo | CD40 agonist mAb (Khalil_DN, 2019) |
| | | Fisher ™, purified for in vivo use) | |
| 4 | OX40 (CD134) | cloneox86 (Bio X Cell ™) | OX40 agonist mAb (Sagiv-Barfi_S, 2022) |
| | agonist | | |
| 5 | CXCL9 | Recombinant Mouse CXCL9/MIG | Chemokine, attracts CXCR3+ Tcells and NK |
| | | R&D Systems (492-MM/CF) | cells; synergizes with IL-12 (Lee_JY, 2022) |
| 6 | IL32γ | Recombinant Human IL-32γ | Mediates macrophage activation/M1 |
| | | Catalog Number: 4690-IL/CF | polarization and enhances cross presentation |
| | | | (Gruber_T, 2020) |

Test ATT-02 (IL-12) monotherapy versus “ATT-02+” in 2-tumor “abscopal” B16-OVA model. The primary endpoint will be tumor growth inhibition of the untreated tumor. Other endpoints includes (1) tumor growth inhibition in treated tumors and total tumor burden; (2) survival; (3) assessment of antigen-specific T cell responses (i.e. SIINFEKL/Kb-tetramer+ CD8s); (5) percentage of TAMs presenting SIINFEKL peptide in the H2-Kb groove (staining with clone 25-D1.16) (6) flow-based immunophenotyping of T cells and NK cells. Attivare has performed experiments testing ATT-02 (IL12+MSRs) as an intralesional therapy in several syngeneic tumor models, including CT26, EMT-6 and in the B16F10 model and in each case, ATT-02 has demonstrated significant tumor growth inhibition of the treated tumors compared to excipient (PBS) and recombinant IL-12.

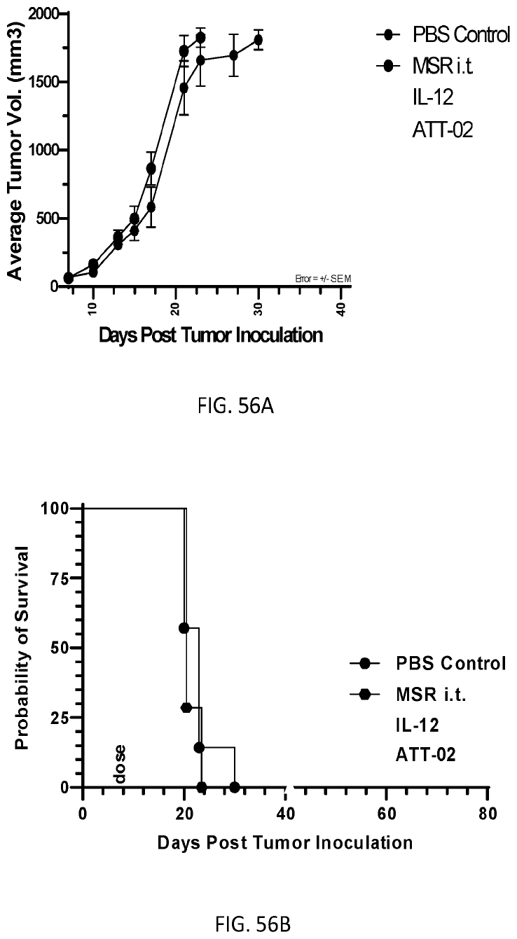

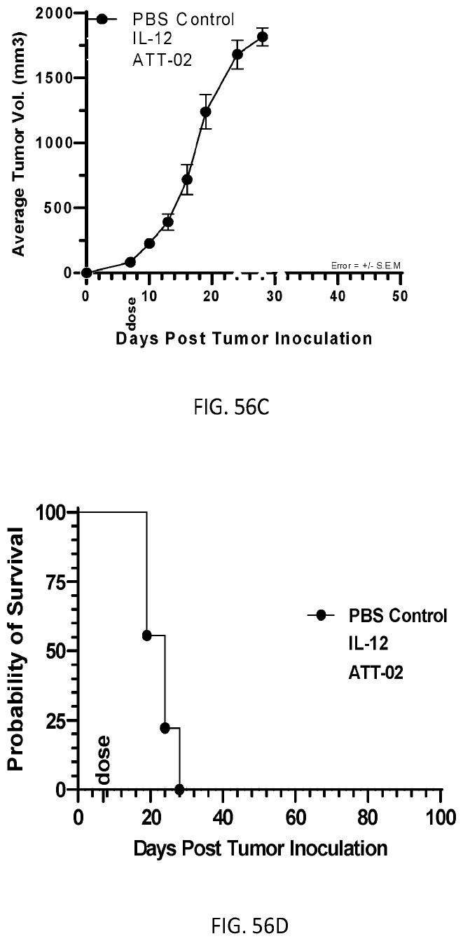

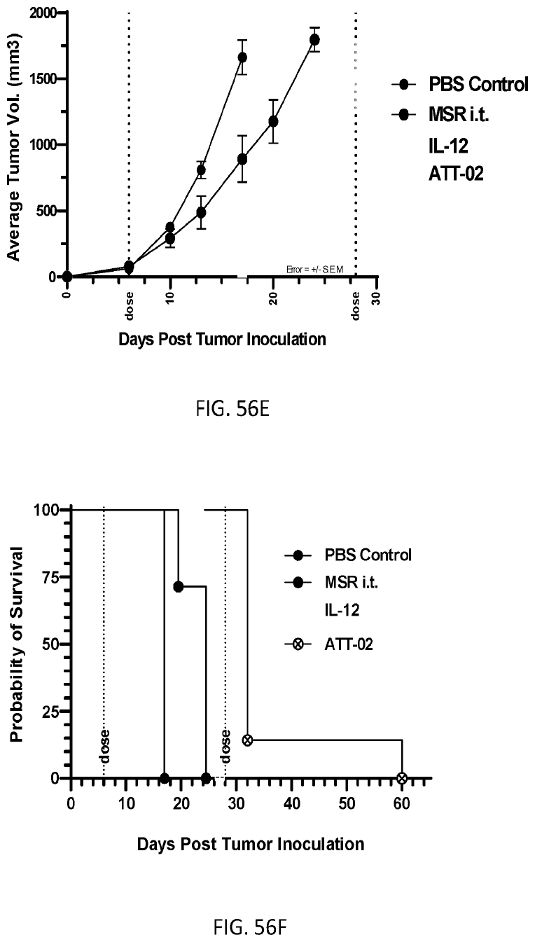

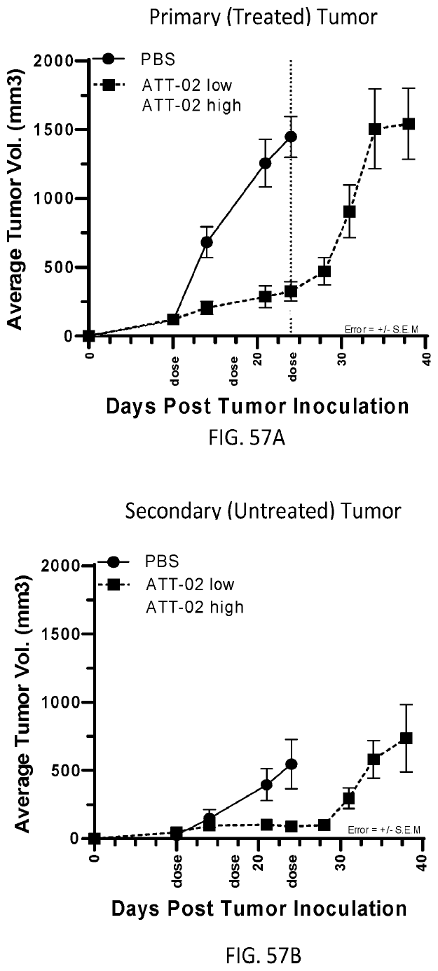

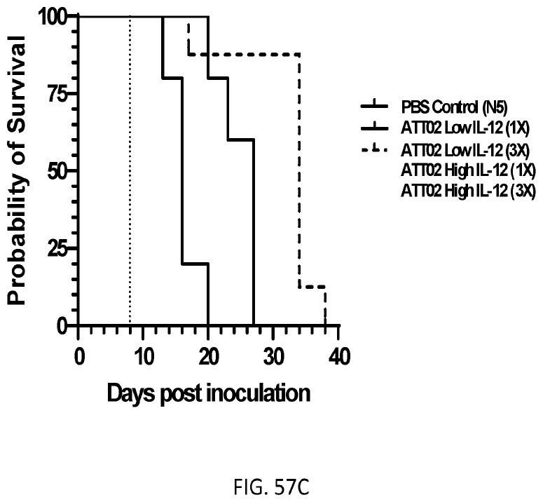

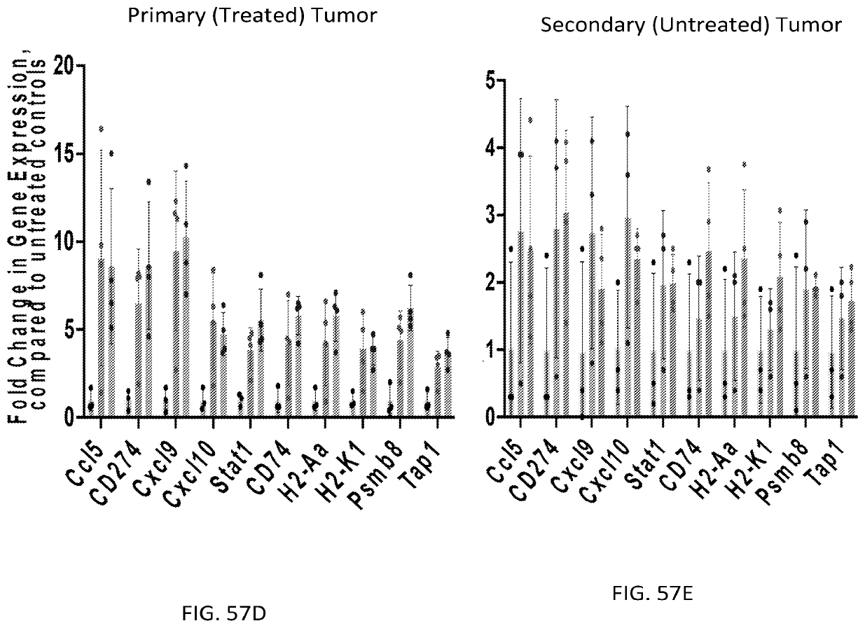

Based on these data, Applicants tested ATT-02 in a difficult-to-treat B16F10 2-tumor “abscopal” model, where only a single tumor is treated and the contralateral tumor is left untreated. In this study, the primary tumor was allowed to grow to approximately 100 mm³ before treatment with ATT-02 was initiated. ATT-02 demonstrated a significant growth inhibition (“abscopal effect”) of both the treated and the untreated tumor, indicating that ATT-02 is capable of generating systemic anti-tumor immune responses (FIG. 57), leading to a significant survival benefit in the ATT-02 treated mice. In this study, treated and untreated tumors from mice treated with ATT-02 at a low dose (28 mg/mg MSR) or high dose (56 mg/mg MSR) were harvested for gene expression analysis at day 7 post-treatment. Both high and low-dose ATT-02 resulted in a significant induction of key INFγ-inducible genes in the treated tumor (FIG. 56D); these genes were also increased, but to a lesser extent, in the distant, untreated tumor at this early timepoint (FIG. 56E).

FIG. 56 shows that incorporation of IL-12 into mesoporous silica rods (MSRs) significantly improves anti-tumor activity and survival compared to recombinant IL-12 alone. Three distinct mouse syngeneic tumor models (CT26 colorectal carcinoma, EMT-6 breast cancer, B16F10 melanoma) were used to investigate the anti-tumor effect of intralesional injection of ATT-02. In general, approximately 1×10⁵-1×10⁶ tumor cells were implanted at a single site in the flank of female mice of the appropriate syngeneic strain and allowed to grow to approximately 50 mm³ in volume prior to initiation of treatment. Tumor volume was measured using digital calipers. Mice were sacrificed when total tumor burden reached 1000-2000 mm³. Significant tumor growth inhibition was observed in all three experimental systems. A clear survival benefit was noted in all three models with complete responses observed in both the CT26 and EMT-6 experiments. (FIG. 56A, FIG. 56B) Mice were inoculated with 1M CT-26 cells subcutaneously in right flank on day 0. On day 7 mice received treatment with PBS, mIL-12 (20 μg, i.t.), MSR (i.t.). (FIG. 56C, FIG. 56D) Mice were inoculated with 1M EMT-6 cells subcutaneously in right flank on day 0. On day 7 mice were dosed with PBS, mIL-12 (20 μg, i.t.), or ATT-02 (i.t, 20 μg IL-12, MSR). (FIG. 56E, FIG. 56F) Mice were inoculated subcutaneously in right flank with 500K B16F10 cells on day 0. On day 6 mice received treatment with PBS, mIL-12 (20 μg, i.t.), ATT-02 (i.t, 20 μg IL-12, MSR), MSR (i.t.). In the CT-26 experiment, 4/7 ATT-02-treated CT26 mice achieved a CR versus 0/7 for IL-12. In the EMT-6 experiment 7/9 mice treated with ATT-02 had a CR versus 3/9 mice treated with IL-12.

In Aim 2, Applicants continued studies with the 2-tumor B16F10 model by employing the related B16-OVA model, in order to facilitate the comparison of ATT-02 (IL-12+MSRs) versus ATT-02+ (best-performing combination from Aim 1) based on the quantitation of antigen-specific CD8 T cell responses (SIINFEKL-tetramer+ CD8 T cells by flow cytometry) as well as tumor growth inhibition and survival. In brief, female C57BL/6 mice age 6-8 weeks received 1.0×10⁶ tumor cells injected into the right flank and, at the same time, a second smaller inoculation (0.25×10⁶ cells) in the left flank. When the larger tumor (right) reached 100 mm³, the mice were randomized into three treatment groups (12 mice/group): PBS control, ATT-02 and ATT-02+. Tumor growth of the primary, treated tumor (right) and contralateral untreated tumor (left) were measured 2-3 times per week using digital calipers, until tumor burden approaches 1000-2000 mm³ or no progression is identified at 45 days. Health checks and body weight were recorded at the time of each tumor measurement. The same experiment was repeated three times to insure the robustness of the tumor growth inhibition. After establishing the reproducibility of the growth inhibition in this model, additional experiments were run to harvest tumors (treated and untreated) at Day 3, 7, 10 for gene expression (Nanostring) to quantify the induction of IFNγ-dependent genes and flow cytometric immunophenotyping of lymphocyte and NK cell populations. Because these tumors express the SIINFEKL epitope of ovalbumin, a strongly immunogenic antigen presented by the H2-Kb MHC class allele which presents SIINFEKL, Applicants can quantify and phenotype tumor-antigen specific CD8 T cell responses systemically (e.g. spleen) and in both the treated and untreated tumor TIL. Additionally, macrophages isolated from disassociated tumors were analyzed for expression of SIINFEKL/Kb complexes and co-stimulatory molecules (e.g. CD80/CD86) indicative of cross-presenting APC. Our analytic approach in Aim 2 largely followed previous published efforts. Terminal bleeds will be used for cytokine measurements (e.g. IL-12, INFγ, CXCL9, CXCL10) and serum markers of liver injury (AST/ALT).

It was expected that ATT-02 would demonstrate significant tumor growth inhibition, corresponding to ATT-02+ significantly outperforming ATT-02 in terms of anti-tumor efficacy (tumor growth inhibition of treated and untreated tumors), including an increase in complete responders and significant improvement in survival.

Further, it was anticipated that the transcriptional analysis of tumor would demonstrate a conversion from an M2 to an M1-dominant TME, including upregulation of APM (antigen processing and presentation machinery), as well as an influx of KLRG1+ SIINFEKL/Kb tetramer+ CD8 T cells. Prism software was used to create Kaplan-Meier survival curves and compared using the log-rank test; other continuous variable endpoints will be compared and assessed for significance using ANOVA. A sample size calculation was performed with the following assumptions: Type 1 error rate (a) of 5%; Power=80%; 2 treatment groups (ATT-02, ATT-02+) and PBS control; 1000 mm³ (mean under HO) and a very conservative estimate of SD (1000) from our prior experiments. Based on this analysis, the recommended group sample size >11.

FIG. 57A-57E shows intralesional ATT-02 drives significant primary and abscopal tumor growth inhibition and increased survival, correlated with induction of an interferon-g-dependent gene signature in the difficult-to-treat B16F10 syngeneic tumor model. Mice were inoculated subcutaneously with B16F10 cells, 1M cells on right-flank (primary) and 0.25M cells on left-flank (secondary) on day 0. On day 10, 17 and 24 mice were dosed with PBS or ATT-02 at two dose levels, low dose (28 μg) or high (56 μg) i.t. in the primary tumor. Nanostring analysis was completed on samples from 7 days post treatment.

Aim 3: Optimize ATT-02+ dose and schedule in an autochthonous MMTV-PyMT mouse mammary tumor model through analysis of (1) the dose/tumor burden/activity relationships, including the immunologic and therapeutic impact of multiple simultaneous and repeat dosing scenarios. A significant stumbling block to the clinical developing of Tavo-EP was the lack of robust preclinical data addressing the dose activity relationships, in particular, whether the fraction of treated tumor mass to overall tumor burden affected the efficacy of the treatment. In this Aim, Applicants utilized the MMTV-PyMT mammary carcinoma mouse model, which is characterized by a high frequency of oncogenesis at an early age (~100% of hemizygous females within 60 days). Because these tumors are multifocal and growth in this autochthonous model is slower than the typical syngeneic cell implantation model, we can better test the hypothesis that (1) multiple simultaneous treatments and/or repeat treatments will increase antitumor T cell activity and significantly slow tumor progression. Cohorts of hemizygous female mice will be monitored twice weekly for tumor growth by palpation and health checks. Tumor growth will be monitored using digital caliper measurements on all palpable tumors once per week. Additionally, 50 μl of blood was sampled from the submandibular vein once per week for quantitation of tumor circulating cell-free DNA (cfDNA) via quantitative PCR for the PyMT transgene as cfDNA in mouse models have been demonstrated to be useful as a measure of tumor burden and response to therapy. Tumor antigen-specific T cell responses directed against the PyMT oncogene will quantitated by flow cytometry using the H-2Dq MT241-250 (LPSLLSNPTY) tetramer as previously reported in conjunction with the KLRG1/“SLEC” flow panel described in Aim2.

SubAim 3.1: To test whether the anti-tumor efficacy of ATT-02+ is dependent on the fraction of tumor burden treated, a cohort of 64 hemizygous female mice will be monitored for tumor development and randomized to four groups of 16 mice receiving a single treatment consisting of either no treatment, injection of ATT-02+ into one tumor, injection of three tumors or injection into all palpable tumors. 8 mice per group were followed for tumor growth kinetics and survival; 4 mice per group will be euthanized after 7 days and 14 days for analysis of the tumor microenvironment for histopathology, multiplex IHC (mIHC) and Nanostring-based gene expression as described in Aim2. Quantitation of tumor antigen (e.g. PyMT 241-250) specific CD8s (i.e. H-2Dq MT241-250 tetramer+) and frequency of KLRG1+CD8 “SLECS” will performed by flow cytometry. Tumor burden was assessed using digital caliper measurements and quantitative PCR for PyMT cfDNA as described above.

SubAim 3.2: This experiment will be analyzed similar in design as SubAim 3.1 except that ATT-02+ will be injected only into a single tumor per treatment. Cohorts of 12 mice will be randomized into 4 groups, receiving (1) no treatment; (2) single dose; (3) two doses one week apart, and (4) three doses at weekly intervals. Anticipated Results/Pitfalls/Considerations: We anticipate that the immunologic activity, tumor growth inhibition and survival benefit of ATT-02+ treatment will be significantly enhanced when a greater fraction of the tumor burden is treated and when tumors are repeatedly dosed. These studies will determine the dose/response relationships and, importantly, whether multiple simultaneous or repeat treatments results in a plateau effect. One obvious caveat to Aim 3 is that the MMTV-PyMT is a breast cancer rather than a melanoma model. It is our contention that—at least in immuno-oncology development—the mechanism of immune dysregulation and the corresponding TME phenotype is more germane than the histogenesis of the malignant cells. For instance, when developing pembrolizumab, our preclinical IND-enabling model was MC38, a carcinogen-induced colon cancer cell line, which produced an inflamed TME replete with PD-1+ exhausted CD8 cells similar to PD-1 responsive melanoma.

Taken together, the multifocality, slower tumor growth rates, the high M2 TAM content and availability of tumor antigen-specific tetramer assays for immune monitoring makes this a useful model to address Aim 3. Milestone: Study completion will help rationalize the dose and schedule strategy to be implemented in our future the Phase 1 clinical trial of ATT-02+.

Successful completion of these studies will determine the components of our IL-12 MSR-based drug candidate (ATT-02 or ATT-02+), provide non-clinical efficacy, preliminary non-GLP safety data as well as help inform the first-in-human dose rationale to support an Investigational New Drug (IND) application for treatment of advanced cutaneous melanoma patients, refractory to anti-PD1 blockade.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present invention, which is defined solely by the claims. Accordingly, the present invention is not limited to that precisely as shown and described.

1. Mesoporous silica rods comprised of a cytokine payload for injection into a tumor or infection,
wherein the cytokine payload is interleukin-12 (IL-12) and/or interleukin-2 (IL-2).

2. (canceled)

3. The mesoporous silica rods of claim 1, further comprised of an adjuvant.

4. The mesoporous silica rods of claim 3, wherein the adjuvant is one or more of aluminum hydroxide (alum), a lipopolysaccharide (LPS) and a toll like receptor agonist (TLR agonist).

5. The mesoporous silica rods of claim 1, wherein the mesoporous silica rods are substantially cylindrical.

6. The mesoporous silica rods of claim 1, wherein the mesoporous silica rods a comprised of an internal labyrinthine structure.

7. A method of treating an ailment, the method comprising:
a) Identifying the ailment in an affected tissue, and
b) inserting the mesoporous silica rod of claim 1 at or near the affected tissue.

8. The method of claim 7, further comprising administration of a therapeutic amount of an anti-PD-1 antibody.

9. The method of claim 7, wherein the ailment is a skin and/or soft tissue infection.

10. The method of claim 9, wherein the infection is a bacterial, fungal or viral infection.

11. The method of claim 7, wherein the ailment is cancer.

12. The method of claim 11, wherein the cancer is at least one of breast, lung, kidney, bladder, urinary tract, urethra, penis, vulva, vagina, cervical, colon, ovarian, prostate, pancreas, stomach, brain, head and neck, skin, uterine, testicular, esophagus, head or neck cancer, brain cancer (e.g., glioma or glioblastoma) and liver cancer.

13. A method of potentiating or stimulating an endogenous immune response to treat an ailment in an affected tissue, the method comprising:
a) Identifying the affected tissue, and
b) inserting mesoporous silica rods at or near the affected tissue.

14. The method of claim 13, wherein the mesoporous silica rods comprise a cytokine payload
wherein the cytokine payload is interleukin-12 (IL-12) and/or interleukin-2 (IL-2).

15. (canceled)

16. The method of claim 13, wherein the mesoporous silica rods further comprise an adjuvant.

17. The method of claim 16, wherein the adjuvant is one or more of aluminum hydroxide (alum), a lipopolysaccharide (LPS) and a toll like receptor agonist (TLR agonist).

18. The method of claim 13, wherein the mesoporous silica rods are substantially cylindrical.

19. The method of claim 13, wherein the mesoporous silica rods comprise an internal labyrinthine structure.

20. The method of claim 13, wherein the affected tissue is tumor tissue or infected tissue.

21.-76. (canceled)
